(19) United States
(12) Patent Application Publication
(10) Pub. No.: US 2026/0183230 A1
(43) Pub. Date: Jul. 2, 2026

(54) Fluid-absorptive gastroretentive dosage form for prolonged drug delivery

(71) Applicant: Dr. Aron H. Blaesi, Cambridge, MA (US)

(72) Inventors: Aron H. Blaesi, Cambridge, MA (US); Nannaji Saka, Cambridge, MA (US)

(73) Assignee: Dr. Aron H. Blaesi, Cambridge, MA (US)

(21) Appl. No.: 19/546,318

(22) Filed: Feb. 21, 2026

Related U.S. Application Data
(63) Continuation of application No. PCT/US2024/043309, filed on Aug. 21, 2024.
(63) Continuation-in-part of application No. PCT/US2024/043307, filed on Aug. 21, 2024.
(63) Continuation-in-part of application No. PCT/US2024/043312, filed on Aug. 21, 2024.

(51) Int. Cl. A61K 9/00; A61K 47/32; A61K 47/38

(52) U.S. Cl. CPC A61K 9/0065; A61K 47/32; A61K 47/38

(57) ABSTRACT

In this specification, anew expandable dosage form is presented where the post-expansion mechanical properties and the drug release rate can be independently controlled. The dosage form generally comprises a three-dimensional structural framework of one or more substantially orderly arranged fluid-absorptive elements, a mechanically strengthening semi-permeable layer substantially encapsulating said fluid-absorptive elements, and a drug-containing solid applied outside said encapsulated elements.

### CROSS-REFERENCE TO RELATED INVENTIONS

This application is a continuation of, and incorporates herein by reference in its entirety, the International Application No. PCT/US2024/043309 filed on Aug. 21, 2024 and titled “Fluid-absorptive gastroretentive dosage form for prolonged drug delivery”, which claims priority to and the benefit of the U.S. Provisional Application No. U.S. 63/533,792 filed on Aug. 21, 2023. Also the foregoing Provisional application is incorporated herein by reference in its entirety.

This application is a continuation-in-part of, and incorporates herein by reference in its entirety, the U.S. application Ser. No. 18/908,569 filed on Oct. 7, 2024 and titled “Gastroretentive fibrous dosage form for prolonged drug delivery”, which is a continuation-in-part of the International Application No. PCT/US2024/043308 filed on Aug. 21, 2024 and titled “Gastroretentive dosage fibrous form for prolonged drug delivery”. Also the foregoing International Application is incorporated herein by reference in its entirety.

This application is also a continuation-in-part of, and incorporates herein by reference in its entirety, the International Application No. PCT/US2024/043307 filed on Aug. 21, 2024 and titled “Gastroretentive dosage form for prolonged drug delivery”.

This application is also a continuation-in-part of, and incorporates herein by reference in its entirety, the International Application No. PCT/US2024/043312 filed on Aug. 21, 2024 and titled “Method for the manufacture of gastroretentive dosage form”.

This application is related to and incorporates herein by reference in their entirety, the U.S. application Ser. No. 15/482,776 filed on Apr. 9, 2017 and titled “Fibrous dosage form”, the U.S. application Ser. No. 15/964,058 filed on Apr. 26, 2018 and titled “Method and apparatus for the manufacture of fibrous dosage forms”, the U.S. application Ser. No. 16/860,911 filed on Apr. 28, 2020 and titled “Expandable structured dosage form for immediate drug delivery”, the U.S. application Ser. No. 16/916,208 filed on Jun. 30, 2020 and titled “Dosage form comprising structural framework of two-dimensional elements”, the U.S. application Ser. No. 17/237,034 filed on Apr. 21, 2021 and titled “Method for 3D-micro-patterning”, the U.S. application Ser. No. 17/327,721 filed on May 23, 2021 and titled “Expandable multi-excipient dosage form”, and the U.S. application Ser. No. 18/124,381 filed on Mar. 21, 2023 and titled “Gastroretentive structured dosage form”, the International Application No. PCT/US19/19004 filed on Feb. 21, 2019 and titled “Expanding structured dosage form”, the International Application No. PCT/US19/52030 filed on Sep. 19, 2019 and titled “Dosage form comprising structured solid-solution framework of sparingly-soluble drug and method for manufacture thereof”, the International Application No. PCT/US21/22857 filed on Mar. 17, 2021 and titled “Expandable, multi-excipient structured dosage form for prolonged drug release”, and the International Application No. PCT/US21/22860 filed on Mar. 17, 2021 and titled “Method and apparatus for 3D-micro-patterning”.

### BACKGROUND OF THE INVENTION

It is well-known that optimal treatment of many kinds of disease requires maintenance of a controlled drug concentration in blood over hours, days, weeks, months, or even years. At present, however, such controlled drug concentration in blood can often times not be achieved.

By way of example but not by way of limitation, numerous drugs that inhibit mutated kinases are fairly effective in treating specific types of cancer, provided the drug concentration in blood is consistently high (see, e.g., D. S. Krause, R. A. van Etten, Tyrosine kinases as targets for cancer therapy, New Engl. J. Med. 353 (2005) 172-187; J. Zhang, P. L. Yang, N. S. Gray, Targeting cancer with small molecule kinase inhibitors, Nature Reviews Cancer 9 (2009) 28-39; and others).

But the drug concentration in blood should not be too high. Such common side effects as prolongation of the Q-t interval, high blood pressure, acute liver damage, headache, and so on, can result when it exceeds a threshold value (see, e.g., J. R. Mitchell, et al., Acetaminophen-induced hepatic necrosis. I. Role of drug metabolism, J. Pharmacol. Exp. Ther. 197 (1973) 185-194; L. Carlsson, et al., Proarrhythmic effects of the class III agent almokalant: importance of infusion rate, QT dispersion, and early after depolarisations, 27 (1993) 2186-2193; D. M. Roden, Drug-induced prolongation of the QT interval, N. Engl. J. Med. 350 (2004) 1013-1022; S. D. Lamore, R. A. Kohnken, M. F. Peters, K. L. Kolaja, Cardiovascular toxicity induced by kinase inhibitors: Mechanisms and preclinical approaches, Chem. Res. Toxicol. 33 (2020) 125-136; and others).

At present, many kinase inhibitors (KIs) are delivered by oral immediate-release tablets or capsules comprising lightly compacted or loose mixtures of drug and excipient particles. A common property of many kinds of KI, however, is that their solubility in gastrointestinal fluid is highly pH-dependent. While slightly soluble in the acidic gastric fluid, they may be virtually insoluble in the pH-neutral intestinal fluid (see, e.g., N. R. Budha, et al., Drug Absorption Interactions Between Oral Targeted Anticancer Agents and PPIs: Is pH-Dependent Solubility the Achilles Heel of Targeted Therapy?, Clinical Pharmacology and Therapeutics 92 (2012) 203-213; B. Herbrink et al., Variability in bioavailability of small molecular tyrosine kinase inhibitors, Cancer Treatment Reviews 41 (2015) 412-422; W. Sun et al., Impact of acid-reducing agents on the pharmacokinetics of palbociclib, a weak base with pH-dependent solubility, with different food intake conditions, Clinical Pharmacology in Drug Development 6 (2017) 614-626).

As shown schematically in the non-limiting FIG. 1a, upon ingestion the KI-containing particulate dosage form may fragment into its constituent particles in the stomach, and the drug particles may dissolve partially. The dissolved drug molecules and the remaining un-dissolved drug particles may then gradually pass into the intestine with the gastric fluid flow, and drug molecules may be absorbed by the blood stream. Thus, as shown in the non-limiting FIG. 1b, the drug concentration in blood may increase.

The drug particles, however, may not dissolve in the intestine. Thus, drug absorption may stop when the drug has been swept out of the stomach, and the drug concentration in blood may decrease, FIG. 1b. Consequently, because the gastric residence time of the drug particles and molecules may be much shorter than the convenient dosing intervals, upon repeated dosing the drug concentration in blood may rise and fall, FIG. 1c. This may therapeutically not be optimal: the maximum drug concentration may be high, promoting acute side effects, and the minimum and average may be low, compromising the efficacy of the therapy.

A steady drug concentration in blood could be achieved by expandable, gastroretentive fibrous dosage forms the present inventors (Blaesi and Saka) have recently introduced (see, e.g., the International Application No. PCT/US21/53027 titled “Gastroretentive structured dosage form”). As shown schematically in FIG. 1d, to facilitate ingestion the dosage forms may be made small enough to pass through the oesophagus rapidly. But as they may expand in the stomach to a size greater than the diameter of the pylorus, their passage into the small intestine may be inhibited. If drug release is slow, the absorption time may be prolonged, and the drug concentration in blood may be fairly steady, FIG. 1e. This may enable higher minimum and average concentrations with lower maximum—enhancing the efficacy of the therapy and mitigating acute side effects, FIG. 1f.

To assure that the expanded fibrous dosage form had adequate mechanical properties for prolonged gastric residence, the prior gastroretentive fibrous dosage forms consisted of drug-laden fibers that were coated with a strengthening enteric excipient, FIG. 2a (see, e.g., A. H. Blaesi, D. Kümmerlen, H. Richter, N. Saka, Mechanical strength and gastric residence time of expandable fibrous dosage forms, Int. J. Pharm. 613 (2022) 120792). However, because the enteric coating may be impermeable, or substantially impermeable, to many drugs, the coating may block drug release, and no or not enough drug may be delivered.

In this specification, therefore, a new dosage form design is presented where the mechanical properties of the expanded dosage form and the drug release rate can be decoupled and independently controlled.

### SUMMARY OF THE INVENTION

Generally, as shown schematically in the non-limiting FIG. 2b, in the invention herein a drug-laden formulation may be applied outside an expandable solid (e.g., water-absorbing fibers coated with a strengthening, enteric excipient, etc.).

More specifically, in one aspect the pharmaceutical solid dosage form disclosed herein comprises a fluid-absorptive, expandable solid core, a mechanically strengthening semi-permeable layer, and a drug-containing solid; said fluid-absorptive, expandable solid core substantially encapsulated by said mechanically strengthening semi-permeable layer; and at least part of said mechanically strengthening semi-permeable layer disposed between said solid core and said drug-containing solid.

In some embodiments, a fluid-absorptive, expandable solid core comprises an expandable three-dimensional structural framework of one or more fluid-absorptive structural elements.

In some embodiments, a structural framework of one or more elements comprises segments spaced apart from adjoining segments by element-free spacings defining one or more element-free spaces within an outer volume of said structural framework.

In some embodiments, moreover, one or more fluid-absorptive elements forming a structural framework is/are substantially orderly arranged.

In another aspect, therefore, the pharmaceutical solid dosage form disclosed herein comprises an expandable, three dimensional structural framework of one or more substantially orderly arranged fluid-absorptive structural elements, a mechanically strengthening semi-permeable layer, and a drug-containing solid; said structural framework of one or more elements comprising segments spaced apart from adjoining segments by element-free spacings defining one or more element-free spaces within an outer volume of said structural framework; said structural framework further substantially encapsulated by said mechanically strengthening semi-permeable layer; and at least part of said mechanically strengthening semi-permeable layer disposed between one or more elements or segments of said structural framework and said drug-containing solid.

In some embodiments, at least one fluid-absorptive structural element comprises a fluid-absorptive fiber.

In some embodiments, one or more element-free spaces within a three dimensional structural framework of one or more fluid-absorptive elements are substantially connected through an exterior dimension of said structural framework.

In some embodiments, moreover, the average element-free spacing through one or more element-free spaces is in the range 10 μm-5 mm.

In some embodiments, a substantially encapsulated structural framework of one or more fluid-absorptive elements comprises substantially encapsulated segments spaced apart from adjoining substantially encapsulated segments by encapsulated-element-free spacings defining one or more encapsulated-element-free spaces within an outer volume of said substantially encapsulated framework.

In some embodiments, moreover, drug-containing solid occupies encapsulated-element-free space.

In some embodiments, one or more encapsulated-element-free spaces within an outer volume of a substantially encapsulated framework of one or more elements further comprise one or more free spaces or one or more channels.

In some embodiments, at least one free space is substantially connected within or through an exterior dimension of said substantially encapsulated structural framework of one or more fluid-absorptive elements.

In some embodiments, the average free spacing through one or more free spaces or channels is in the range 5 μm-3 mm.

In some embodiments, drug-containing solid occupies encapsulated-element-free space between a free space or channel and an encapsulated element or segment of said substantially encapsulated three-dimensional structural framework.

In some embodiments, drug-containing solid is bonded or attached to a mechanically strengthening semi-permeable layer forming at least part of said substantially encapsulated structural framework of one or more elements.

In some embodiments, the composition of a fluid-absorptive element (e.g., a fluid-absorptive core, etc.) includes at least a fluid-absorptive first excipient in which the solubility of gastric fluid is greater than 400 mg/ml (e.g., greater than 450 mg/ml, greater than 500 mg/ml, greater than 550 mg/ml, greater than 600 mg/ml, greater than 650 mg/ml, greater than 700 mg/ml, greater than 750 mg/ml) under physiological conditions.

In some embodiments, the composition of a fluid-absorptive element includes hydroxypropyl methylcellulose.

In some embodiments, the composition of a fluid-absorptive element includes at least a fluid-absorptive first excipient, said fluid-absorptive first excipient selected from the group consisting of hydroxypropyl methylcellulose, hydroxyethyl cellulose, polyvinyl alcohol, polyvinylpyrrolidone, sodium alginate, hydroxypropyl cellulose, hydroxyethyl cellulose, methyl cellulose, hydroxypropyl methyl ether cellulose, starch, polymethacrylates (e.g., poly(methacrylic acid, ethyl acrylate) 1:1, or butylmethacrylat-(2-dimethylaminoethyl)methacrylat-methylmathacrylat-copolymer), polyethylene oxide, xanthan gum, or vinylpyrrolidone-vinyl acetate copolymer.

In some embodiments, a mechanically strengthening semi-permeable layer forms a viscoelastic, semi-permeable membrane upon exposure to or soaking with gastric fluid under physiological conditions, said viscoelastic, semi-permeable membrane being substantially permeable to gastric fluid under physiological conditions.

In some embodiments, moreover, a mechanically strengthening semi-permeable layer forms a viscoelastic, semi-permeable membrane upon exposure to or soaking with gastric fluid under physiological conditions, said viscoelastic, semi-permeable membrane being substantially impermeable to at least a gastric fluid-absorptive first excipient included in one or more gastric fluid-exposed or gastric fluid-soaked structural elements.

In some embodiments, a mechanically strengthening semi-permeable layer comprises a tensile strength greater than 0.05 MPa (e.g., greater than 0.075 MPa, greater than 0.1 MPa, greater than 0.2 MPa, greater than 0.3 MPa, greater than 0.4 MPa, greater than 0.5 MPa, etc.) after soaking with gastric fluid under physiological conditions.

In some embodiments, the composition of a mechanically strengthening semi-permeable layer comprises at least a strengthening second excipient, said strengthening second excipient comprising a solubility no greater than 1 g/l (e.g., no greater than 0.075 g/l, no greater than 0.05 g/l, etc.) in gastric fluid under physiological conditions.

In some embodiments, the composition of a mechanically strengthening semi-permeable layer comprises at least a strengthening second excipient, and solubility of gastric fluid in said strengthening second excipient being no greater than 700 mg/ml under physiological conditions.

In some embodiments, the composition of a mechanically strengthening semi-permeable layer comprises methacrylic acid-ethyl acrylate copolymer.

In some embodiments, the composition of a mechanically strengthening semi-permeable layer comprises at least a strengthening second excipient, said strengthening second excipient selected from the group consisting of methacrylic acid-ethyl acrylate copolymer, methacrylic acid-methyl methacrylate copolymer, ethyl acrylate-methylmethacrylate copolymer, hydroxypropyl methylcellulose acetate succinate, polyvinyl acetate, polymers including methacrylic acid, polymers including ethyl acrylate, polymers including methyl methacrylate, polymers including methacrylate, Poly[Ethyl acrylate, methyl methacrylate, trimethylammonioethyl methacrylate chloride], and ethylcellulose.

In some embodiments, a drug containing solid comprises at least an active pharmaceutical ingredient or drug dispersed as drug particles and/or as drug molecules in one or more third excipients, said one or more third excipients comprising at least one excipient having a solubility greater than 1 mg/ml (e.g., greater than 2 mg/ml, greater than 5 mg/ml, greater than 10 mg/ml, etc.) in gastric fluid under physiological conditions and forming a matrix that erodes upon exposure to said gastric fluid.

In some embodiments, at least one third excipient that is soluble in gastric fluid under physiological conditions comprises hydroxypropyl methylcellulose.

In some embodiments, at least one third excipient that is soluble in gastric fluid under physiological conditions is selected from the group consisting of hydroxypropyl methylcellulose, hydroxyethyl cellulose, polyvinyl alcohol, polyvinylpyrrolidone, sodium alginate, hydroxypropyl cellulose, hydroxyethyl cellulose, methyl cellulose, hydroxypropyl methyl ether cellulose, starch, polymethacrylates (e.g., poly(methacrylic acid, ethyl acrylate) 1:1, or butylmethacrylat-(2-dimethylaminoethyl)methacrylat-methylmathacrylat-copolymer), polyethylene oxide, or vinylpyrrolidone-vinyl acetate copolymer.

In some embodiments, a free space or channel is filled with a matter selected from the group comprising gas, liquid, or solid, or combinations thereof, and wherein said matter is partially or entirely removed upon contact with gastric fluid under physiological conditions.

In another aspect, the pharmaceutical solid dosage form disclosed herein comprises a fluid-absorptive, expandable solid core comprising at least a first pharmaceutical excipient, a mechanically strengthening semi-permeable layer comprising at least a second pharmaceutical excipient, and a drug-containing solid comprising at least an active ingredient and at least a third pharmaceutical excipient; said fluid-absorptive, expandable solid core substantially encapsulated by said mechanically strengthening semi-permeable layer; and at least part of said mechanically strengthening semi-permeable layer disposed between said solid core and said drug-containing solid; wherein said first excipient is substantially mutually soluble with gastric fluid under physiological conditions and selected from the group consisting of hydroxypropyl methylcellulose, hydroxyethyl cellulose, polyvinyl alcohol, polyvinylpyrrolidone, sodium alginate, hydroxypropyl cellulose, hydroxyethyl cellulose, methyl cellulose, hydroxypropyl methyl ether cellulose, starch, polymethacrylates, poly(methacrylic acid, ethyl acrylate) 1:1, butylmethacrylat-(2-dimethylaminoethyl)methacrylat-methylmathacrylat-copolymer, polyethylene oxide, xanthan gum, or vinylpyrrolidone-vinyl acetate copolymer; the solubility of gastric fluid in said second excipient is no greater than 700 mg/ml, and said second excipient is selected from the group consisting of methacrylic acid-ethyl acrylate copolymer, methacrylic acid-methyl methacrylate copolymer, ethyl acrylate-methylmethacrylate copolymer, hydroxypropyl methylcellulose acetate succinate, polyvinyl acetate, polymers including methacrylic acid, polymers including ethyl acrylate, polymers including methyl methacrylate, polymers including methacrylate, Poly[Ethyl acrylate, methyl methacrylate, trimethylammonioethyl methacrylate chloride], and ethylcellulose; and said third excipient comprises a solubility in gastric fluid greater than 1 mg/ml under physiological conditions and is selected from the group consisting of hydroxypropyl methylcellulose, hydroxyethyl cellulose, polyvinyl alcohol, polyvinylpyrrolidone, sodium alginate, hydroxypropyl cellulose, hydroxyethyl cellulose, methyl cellulose, hydroxypropyl methyl ether cellulose, starch, polymethacrylates, poly(methacrylic acid, ethyl acrylate) 1:1, butylmethacrylat-(2-dimethylaminoethyl)methacrylat-methylmathacrylat-copolymer, polyethylene oxide, or vinylpyrrolidone-vinyl acetate copolymer; and upon immersing said pharmaceutical solid dosage form to gastric fluid under physiological conditions, the substantially encapsulated solid core expands with fluid absorption and said drug-containing solid releases said active ingredient over time.

### BRIEF DESCRIPTION OF THE DRAWINGS

The objects, embodiments, features, and advantages of the present invention may be more fully understood when considered in conjunction with the following accompanying drawings:

FIG. 1 presents non-limiting schematics of (a) the passage of an immediate-release particulate dosage form through the gastrointestinal tract, (b) drug concentration in blood after administering a single particulate dosage form, (c) drug concentration in blood after repeated dosing of particulate dosage forms, (d) the passage of a gastroretentive fibrous dosage form through the gastrointestinal tract, (e) drug concentration in blood after administering a single gastroretentive fibrous dosage form, and (f) drug concentration in blood after repeated dosing of gastroretentive fibrous dosage forms. c_{d,b}: drug concentration in blood; cₘₐₓ: maximum drug concentration in blood; t: time; t_{d}: dosing interval; w_{1/2}: width of peak at half height (e.g., width of peak drug concentration at half height of the peak);

FIG. 2 presents non-limiting conceptual designs of gastroretentive dosage forms: (a) water-absorbing, drug-laden fibers surrounded by an enteric coating, and (b) an expandable solid (e.g., water-absorbing, enteric-excipient-coated fibers, etc.) surrounded by a drug-laden formulation;

FIG. 3 presents a non-limiting example of a pharmaceutical solid dosage form according to this invention;

FIG. 4 schematically shows another non-limiting example of a pharmaceutical solid dosage form according to this invention;

FIG. 5 presents another non-limiting example of a pharmaceutical solid dosage form according to this invention;

FIG. 6 schematically shows a non-limiting example of a state-of-the-art immediate release particulate dosage form;

FIG. 7 presents another non-limiting example of a pharmaceutical solid dosage form according to this invention, wherein FIG. 7a is a non-limiting example of the top view and FIG. 7b a non-limiting example of the sectional view of the microstructure of the dosage form;

FIG. 8 illustrates a drug particle exposed to a free-flowing dissolution fluid. The black dots represent dissolved drug molecules. cₛ: solubility of drug in dissolution fluid, Rₚ(t): radius of drug particle at time, t, v_{∞,p}: far-field velocity of dissolution fluid relative to drug particle, δ_{c}: drug concentration boundary layer thickness;

FIG. 9 shows non-limiting schematics of the microstructure of a fibrous dosage form according to this invention, the free-body diagram of a half-section of an expanding fiber, and the concentration of water in the fiber and the inter-fiber space versus radial distance: (a) immediately and (b) at time t after immersing the fibrous dosage form in a dissolution fluid. c_{w}: water concentration, h_{ec}: thickness of enteric coating in expanding/expanded dosage form, h_{ec,0}: thickness of enteric coating in solid dosage form, R_{f}: fiber radius in expanding/expanded dosage form, R_{f,0}: fiber radius in solid dosage form, x: axial coordinate, Π: osmotic pressure, ρ_{w}: density of water, σ_{θ}: tensile stress;

FIG. 10 presents non-limiting schematics of the erosion of an HPMC1-enteric excipient solid solution from drug-laden annuli: (a) solid annulus and its microstructure before immersing the dosage form in the dissolution fluid, (b) separation of the excipient solid solution into two phases after immersion, and (c) erosion of the excipient matrix;

FIG. 11 shows non-limiting schematics of a fluid-filled channel and the HPMC1 concentration profiles in the channel for small velocities of dissolution fluid through the channel, v_{z};

FIG. 12 illustrates (a) selected anatomy of the human body, and (b) a non-limiting flow diagram of the passage of drug through the body after ingesting a dosage form;

FIG. 13 shows non-limiting schematics of drug in the stomach after administering particulate dosage forms: (a) t<t_{r,p} and (b) t>>t_{r,p}. Here t_{r,p} is the “residence time” of drug particles in the stomach;

FIG. 14 is a non-limiting plot of the molecular concentration of drug in the gastric fluid, c_{d,gf}, versus time, t, after administering the particulate dosage form. The drug concentrations are calculated by Eqs. (12) and (13) using the non-limiting parameter values given in the text;

FIG. 15 is a non-limiting schematic of the expansion of a fibrous dosage form in the gastric fluid: (a) solid dosage form immediately after ingestion, and (b) expanded dosage form. h_{ec,0}: thickness of solid coating, R_{f,0}: fiber radius, Π₀: osmotic pressure in fiber at “zero” expansion, σ_{θ}: hoop stress in fiber coating;

FIG. 16 presents a non-limiting schematic of a fibrous dosage form exposed to cyclic loading. a: semi-width of contact; Pₐ: applied load intensity (load per unit length); P_{a,max}: maximum applied load intensity due to contracting stomach walls; R_{df}: radius of expanded dosage form; tₚᵤₗₛₑ: period of contraction pulse; σₐ: tensile stress due to applied load; σ_{a,max}: maximum tensile stress due to contracting stomach walls;

FIG. 17 is a non-limiting schematic illustrating drug release by gastroretentive fibrous dosage form in the gastric fluid;

FIG. 18 is a non-limiting plot of drug concentration in gastric fluid, c_{d,gf}, versus time, t, after administering a fibrous dosage form. The drug concentrations are calculated by Eqs. (19) and (20) using the parameter values given in the text. The curve of the particulate form is from FIG. 14;

FIG. 19 is a non-limiting schematic illustrating drug absorption in the duodenum by diffusion across the epithelial membrane into the duodenal capillaries;

FIG. 20 presents a non-limiting schematic of drug distribution from blood capillary into tissue;

FIG. 21 shows a non-limiting schematic of drug elimination from the sinusoids of the liver by drug diffusion across the hepatic plates into the biliary canaliculi;

FIG. 22 presents a non-limiting simplified lumped parameter model of drug release, absorption, drug distribution, and drug elimination after administering a dosage form. The volume of the tissue is the sum of the tissue volumes of all organs, including the stomach, duodenum, liver, heart, and so on;

FIG. 23 is a non-limiting plot of the calculated drug concentration in blood, c_{d,b}, versus time, t, after administering a particulate dosage form. The drug concentration is calculated by Eqs. (27) and (28) using the non-limiting parameter values given in the text;

FIG. 24 is a non-limiting plot of the calculated drug concentration in blood, c_{d,b}, versus time, t, after administering fibrous and particulate dosage forms. The concentration of the fibrous form is from Eqs. (29) and (30) using the non-limiting parameter values given in the text. The curve of the particulate form is from FIG. 23;

FIG. 25 presents a non-limiting example of a structure of a fluid-absorptive, expandable solid core herein;

FIG. 26 presents a non-limiting example of a structure comprising criss-crossed stacked layers of one or more fibers and a histogram of the inter-fiber distance;

FIG. 27 shows a non-limiting example of a dosage form (or an expandable, gastroretentive solid) herein comprising a fluid-absorptive solid core and a mechanically strengthening, semi-permeable layer;

FIG. 28 shows another non-limiting example of a pharmaceutical dosage form herein;

FIG. 29 presents a non-limiting example of a drug-containing solid herein and its drug release mechanism after immersing in a relevant dissolution fluid (e.g., gastric fluid). c₀: concentration of soluble excipient at solid-liquid interface (e.g., solubility or disentanglement concentration of soluble excipient), δ_{c}: concentration boundary layer thickness, H: thickness of drug-containing solid;

FIG. 30 shows a scanning electron micrograph of a mixture of drug and excipient particles of a particulate dosage form. The volume-based average particle radius was about 18.5 μm;

FIG. 31 depicts scanning electron micrographs of fibrous dosage forms: (a) top view and (b) longitudinal section of uncoated fibrous cylindrical disk; (c) top view and (d) longitudinal section of coated fibrous cylindrical disk; (e) top view and (f) cross section of final dosage form;

FIG. 32 depicts (a) top-view images of a particulate dosage form and (b) a fibrous dosage form at various times after immersing in a dissolution fluid.

FIG. 33 plots (a) normalized radial expansion of a fibrous dosage form, ΔR_{df}/R_{df,0}, versus time, t, after immersing in a dissolution fluid, and (b) measured normalized radial expansion, ΔR_{df}/R_{df,0|meas} versus 2π₀φ_{f}t/φ_{ec}η. The measured values are from FIG. 32b. Calculated values of 2π₀φ_{f}t/φ_{ec}η are obtained using the non-limiting parameters given in the text at the times the data were acquired;

FIG. 34 depicts images of the diametral compression test of an expanded fibrous dosage form at different times during loading. The dosage form was soaked in a dissolution fluid for 10 hours before the experiment;

FIG. 35 plots data of diametral compression of fibrous dosage forms: (a) Load intensity, P. versus displacement, δ, and (b) dP/dδ versus δ. P is the force per unit thickness of the expanded dosage form. The experiments were conducted 10 hours after immersing the dosage forms in the dissolution fluid. The thickness of the expanded dosage forms was about 12 mm;

FIG. 36 plots experimental results of the fraction of drug released after immersing (a) a particulate and (b) a fibrous dosage form in a dissolution fluid;

FIG. 37 depicts the position and shape of a particulate dosage form after administering to a fasted dog. The images were obtained by biplanar fluoroscopy. They show the abdomen in lateral projection (cranial left, caudal right);

FIG. 38 presents the position and shape of a fibrous dosage form after administration to a fasted dog. Dry food was given 3 h after administration. The images were obtained by biplanar fluoroscopy. They show the abdomen in lateral projection (cranial left, caudal right);

FIG. 39 plots calculated and measured normalized radial expansion of fibrous dosage forms, ΔR_{df}/R_{df,0}, versus time, t. The calculated curve is obtained from Eq. (3) using the parameter values of Appendix A. The in vivo data represent two samples administered to two dogs. They are obtained from fluoroscopic images of the abdomen of the dog as illustrated in FIG. 38. The in vitro data are from FIG. 33;

FIG. 40 depicts fluoroscopic image sequences of a fibrous dosage form during a compression pulse by the stomach walls of a dog 6 hours after administration;

FIG. 41 plots measured and calculated drug concentrations in the blood plasma of dogs after administering (a) particulate and (b) fibrous dosage forms. The calculated values (also shown in FIGS. 24 and 25) are obtained from Eqs. (27a)-(30) using the parameter values given in the text. (The drug concentration in the blood plasma is assumed the same as the drug concentration in blood.) The measured values represent the average of four particulate dosage forms and two fibrous dosage forms.

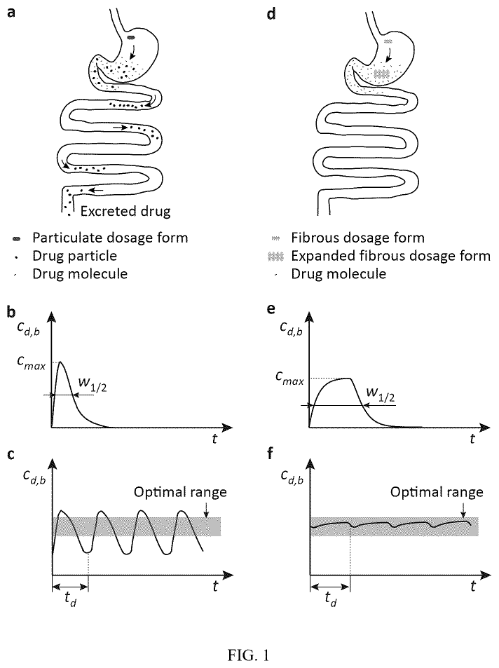

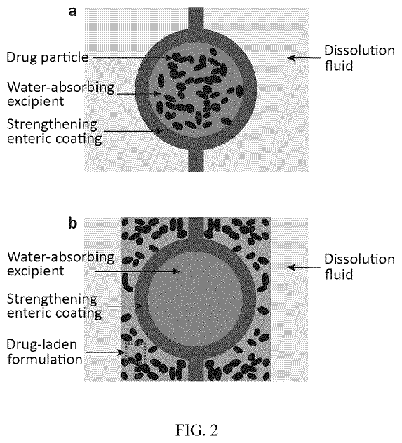

### DEFINITIONS

In order for the present disclosure to be more readily understood, definitions for certain terms are suggested below. Additional definitions for the following terms and other terms are set forth throughout the specification. It may be noted that the definitions are not meant to be limited in any way.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art.

Moreover, in the disclosure herein, the terms “active ingredient”, “one or more active ingredients”, “active pharmaceutical ingredient”, “one or more active pharmaceutical ingredients”, “drug”, “one or more drugs”, and so on are used interchangeably. As used herein, an “active ingredient” or “active agent” or “drug” refers to an agent whose presence or level correlates with elevated level or activity of a target, as compared with that observed absent the agent (or with the agent at a different level). In some embodiments, an active ingredient is one whose presence or level correlates with a target level or activity that is comparable to or greater than a particular reference level or activity (e.g., that observed under appropriate reference conditions, such as presence of a known active agent, e.g., a positive control).

In the invention herein, a drug-containing solid generally comprises a solid that includes or contains at least a drug. A drug-containing solid generally can have any shape, geometry, or form.

Furthermore, in the context of some embodiments herein, a three dimensional structural framework (or network) of one or more elements comprises a structure (e.g., an assembly or an assemblage or an arrangement or a skeleton or a skeletal structure or a three-dimensional lattice structure of said one or more elements) that may extend over a length, width, and thickness greater than 100 μm. This includes, but is not limited to structures that extend over a length, width, and thickness greater than 200 μm, or greater than 300 μm, or greater than 500 μm, or greater than 700 μm, or greater than 1 mm, or greater than 1.25 mm, or greater than 1.5 mm, or greater than 2 mm.

In other embodiments, a three dimensional structural framework (or network) of elements may comprise a structure (e.g., an assembly or an assemblage or a skeleton or a skeletal structure or a lattice structure of said elements) that extends over a length, width, and thickness greater than the average thickness of at least one element (or at least one segment) in the three dimensional structural framework (or network) of elements. This includes, but is not limited to structures that extend over a length, width, and thickness greater than 1.5, or greater than 2, or greater than 2.5, or greater than 3, or greater than 3.5, or greater than 4 times the average thickness of at least one element (or at least one segment) in the three dimensional structural framework (or network) of elements.

In some embodiments, a three dimensional structural framework (or network) of elements is continuous. In other words, in some embodiments a three dimensional structural framework (or network) of elements forms a continuous structure. Furthermore, in some embodiments, one or more elements or segments thereof are bonded to each other or interpenetrating.

It may be noted that the terms “three dimensional structural network”, “structural network”, “three dimensional structural framework”, “structural framework”, “framework”, and “three dimensional lattice structure” are used interchangeably herein. Also, the terms “three dimensional structural framework of elements”, “three dimensional structural framework of one or more elements”, “three dimensional structural framework of one or more fibers”, “three dimensional fiber structural framework”, “three dimensional fibrous structural framework”, “fibrous structural framework”, “fiber structural framework”, “three dimensional structural framework of fibers”, “three dimensional framework”, “structural framework”, etc. are used interchangeably herein.

In the invention herein, a “structural element” or “element” refers to a two-dimensional element (or 2-dimensional structural element), or a one-dimensional element (or 1-dimensional structural element), or a zero-dimensional element (or 0-dimensional structural element). In the invention herein, moreover, the terms “one or more elements” and “elements” are generally used interchangeably.

As used herein, a two-dimensional structural element is referred to as having a length and width much greater than its thickness. In the present disclosure, the length and width of a two-dimensional structural element are greater than 2 times its thickness. An example of such an element is a “sheet”. A one-dimensional structural element is referred to as having a length much greater than its width and thickness. In the present disclosure, the length of a one-dimensional structural element is greater than 2 times its width and thickness. An example of such an element is a “fiber”. A zero-dimensional structural element is referred to as having a length and width of the order of its thickness. In the present disclosure, the length and width of a zero-dimensional structural element are no greater than 2 times its thickness. Furthermore, the thickness of a zero-dimensional element is less than 2.5 mm. Examples of such zero-dimensional elements are “particles” or “beads” and include polyhedra, spheroids, ellipsoids, or clusters thereof.

Moreover, in the invention herein, a segment of a one-dimensional element is a fraction of said element along its length. A segment of a two-dimensional element is a fraction of said element along its length and/or width. A segment of a zero-dimensional element is a fraction of said element along its length and/or width and/or thickness. The terms “segment of a one-dimensional element”, “fiber segment”, “segment of a fiber”, and “segment” are used interchangeably herein. Also, the terms “segment of a two-dimensional element” and “segment” are used interchangeably herein. Also, the terms “segment of a zero-dimensional element” and “segment” are used interchangeably herein.

As used herein, the terms “fiber”, “fibers”, and “one or more fibers”, are used interchangeably. They are understood as the solid, structural elements (or building blocks) that can make up part of a three dimensional structural framework or network or an entire three dimensional structural framework or network. A fiber may have a length much greater than its width and thickness. In the present disclosure, a fiber is referred to as having a length greater than 2 times its width and thickness (e.g., the length is greater than 2 times the fiber width and the length is greater than 2 times the fiber thickness). This includes, but is not limited to a fiber length greater than 3 times, or greater than 4 times, or greater than 5 times, or greater than 6 times, or greater than 8 times, or greater than 10 times, or greater than 12 times the fiber width and thickness. In other embodiments that are included but not limiting in the disclosure herein, the length of a fiber may be greater than 0.3 mm, or greater than 0.5 mm, or greater than 1 mm, or greater than 2.5 mm.

Moreover, as used herein, the term “fiber segment” or “segment” refers to a fraction of a fiber along the length of said fiber.

In the invention herein, fibers (or fiber segments) may be bonded, and thus they may serve as building blocks of “assembled structural elements” with a geometry different from that of the original fibers. Such assembled structural elements include two-dimensional elements, one-dimensional elements, or zero-dimensional elements.

In the invention herein, a “layer” or a “ply” of one or more fibers refers to a layer or ply formed by at least two fibers or at least two fiber segments in a plane defining said layer or ply. This includes, but is not limited to a layer or ply formed by at least three fibers or at least three fiber segments, or least four fibers or at least four fiber segments in a plane defining said layer or ply.

In the invention herein, an “expandable solid” refers to a solid or semi-solid or viscoelastic material that expands upon immersing in a relevant physiological fluid under physiological conditions. A solid or semi-solid or viscoelastic material is referred to as “expanding upon immersing in a relevant physiological fluid under physiological conditions” if it has at least one exterior dimension expanded to greater than its length prior to immersing in said physiological fluid.

In some embodiments of the invention herein, an expandable solid is also a gastroretentive solid. In such embodiments, the terms “expandable solid”, “gastroretentive solid”, “gastroretentive, expandable solid”, and “expandable, gastroretentive solid” are used interchangeably herein.

As used herein, a “gastroretentive solid” refers to a solid or semi-solid or viscoelastic material which upon ingestion by a human subject (or an animal subject that reasonably resembles a human subject) resides in the stomach longer than a small particle that is substantially insoluble in gastric fluid. This includes, but is not limited to a solid or semi-solid or viscoelastic material which upon ingestion by a human subject (or an animal subject that reasonably resembles a human subject) resides in the stomach substantially longer than a small particle that is substantially insoluble in gastric fluid. This also includes, but is not limited to a solid or semi-solid or viscoelastic material which upon ingestion by a human subject (or an animal subject that reasonably resembles a human subject) resides in the stomach at least 1.2 (e.g., at least 1.4, or at least 1.6, or at least 1.8, or at least 2, or at least 3, or at least 4, or at least 5) times as long as a small particle that is substantially insoluble in gastric fluid. By way of example but not by way of limitation, a small particle may comprise a substantially insoluble, biologically inert particle with a size of the order of 1 mm-5 mm or similar.

As used herein, the terms “physiological fluid”, “body fluid”, “dissolution medium”, “dissolution fluid”, “medium”, “fluid”, “aqueous solution”, “fluid”, “penetrant”, etc. are used interchangeably. They are understood as any fluid produced by or contained in a human body under physiological conditions, or any fluid that resembles a fluid produced by or contained in a human body under physiological conditions. Generally, a dissolution fluid contains water and thus may be aqueous. Examples include, but are not limited to: water, saliva, stomach fluid, gastrointestinal fluid, saline, simulated gastric fluid, etc. at a temperature of 37° C. and a pH value adjusted to the relevant physiological condition.

In the invention herein, moreover, a “relevant physiological fluid” is understood as the relevant physiological fluid surrounding the dosage form in the relevant physiological application. For example, if the dosage form is a gastroretentive dosage form, a relevant physiological fluid is gastric fluid or a fluid that resembles gastric fluid.

As used herein, moreover, the terms “gastric fluid”, “fluid that resembles gastric fluid”, “simulated gastric fluid”, “acidic water”, and so on are used interchangeably. They refer to gastric fluid or a fluid that resembles gastric fluid. A fluid that resembles gastric fluid is generally understood herein as acidic water at a pH in the range of about 1-2 and a temperature of about 37° C. This includes, but is not limited to acidic water at a pH of about 1.5 and a temperature of about 37° C. This also includes, but is not limited to a mixture of water and hydrochloric acid at a pH in the range of 1-2 and a temperature of about 37° C. This also includes, but is not limited to a mixture of water and hydrochloric acid at a pH of 1.5 and a temperature of about 37° C. Generally, moreover, a fluid that resembles gastric fluid may be stirred.

In the invention herein, moreover, the terms “intestinal fluid”, “fluid that resembles intestinal fluid”, “simulated intestinal fluid”, and so on are used interchangeably. They refer to intestinal fluid or a fluid that resembles intestinal fluid. A fluid that resembles intestinal fluid is generally understood herein as water at a pH in the range of about 6-7.5 and a temperature of about 37° C. This includes, but is not limited to an aqueous buffer solution at a pH in the range of 6-7.5 and a temperature of about 37° C. This also includes, but is not limited to an aqueous buffer solution at a pH of about 6.8 and a temperature of about 37° C. This also includes, but is not limited to an aqueous buffer solution at a pH of about 7.2 and a temperature of about 37° C. Generally, moreover, a fluid that resembles intestinal fluid may be stirred.

Furthermore, in the invention herein, a fluid-absorptive solid, such as a fluid-absorptive solid core, a fluid-absorptive core, a fluid-absorptive structural framework, a fluid-absorptive structural element, a fluid-absorptive element, a fluid-absorptive fiber, and so on, may generally comprise at least a fluid-absorptive excipient.

In the invention herein, a “fluid-absorptive excipient” is referred to as an excipient that is “absorptive” of gastric or a relevant physiological fluid under physiological conditions. Generally, said absorptive excipient is a solid, or a semi-solid, or a viscoelastic material in the dry state at room temperature. Upon contact with (e.g., immersion in) gastric or a relevant physiological fluid under physiological conditions, however, said fluid-absorptive excipient can absorb said fluid and form solutions or mixtures with said fluid having a weight fraction of gastric or relevant physiological fluid greater than 0.4. This includes, but is not limited to the formation of solutions or mixtures with a weight fraction of gastric or relevant physiological fluid greater than 0.5, or greater than 0.6, or greater than 0.7, or greater than 0.75, or greater than 0.8, or greater than 0.85, or greater than 0.9, or greater than 0.95. In other words, the solubility of gastric fluid or a relevant physiological fluid in the fluid-absorptive excipient under physiological conditions generally is greater than about 400 mg/ml. This includes, but is not limited to solubility of gastric or relevant physiological fluid in an absorptive excipient greater than 500 mg/ml, or greater than 600 mg/ml, or greater than 700 mg/ml, or greater than 750 mg/ml, or greater than 800 mg/ml, or greater than 850 mg/ml, or greater than 900 mg/ml, or greater than 950 mg/ml. It may be noted that the terms “fluid-absorptive”, “physiological fluid-absorptive”, “fluid-absorbing”, “physiological fluid-absorbing”, “water-absorptive”, “water-absorbing”, “gastric fluid-absorptive”, “gastric fluid-absorbing”, “absorptive”, “absorbing”, and so on are generally used interchangeably herein.

It may be noted, furthermore, that an absorptive excipient may also be highly soluble in a physiological fluid.

Preferably, an absorptive excipient may be mutually soluble with a relevant physiological fluid under physiological conditions, such as gastric fluid. By way of example but not by way of limitation, an absorptive excipient may be mutually soluble with a relevant physiological fluid (e.g., with a relevant physiological fluid under physiological conditions) in all proportions. Non-limiting examples of preferred absorptive excipients may include, but are not limited to water-soluble polymers of large molecular weight and with amorphous molecular structure, such as hydroxypropyl methylcellulose with a molecular weight (e.g., a number-average molecular weight) greater than 50 kg/mol or hydroxypropyl methylcellulose with a molecular weight (e.g., a number-average molecular weight) in the range between 50 kg/mol and 1000 kg/mol.

Similarly, in the invention herein, a mechanically strengthening phase may generally comprise at least a mechanically strengthening excipient.

In the invention herein, a “strengthening excipient”, too, may generally be a solid, or a semi-solid, or a viscoelastic material in the dry state at room temperature. Upon contact with (e.g., immersion in, etc.) gastric or a relevant physiological fluid under physiological conditions, however, said strengthening excipient may be far less absorptive of said fluid, and thus it may remain a solid, or semi-solid, or viscoelastic, or highly viscous material. Generally, the solubility of gastric or relevant physiological fluid in strengthening excipient under physiological conditions may be no greater than 800 mg/ml. This includes, but is not limited to a solubility of gastric or a relevant physiological fluid in strengthening excipient under physiological conditions no greater than 750 mg/ml, or no greater than 700 mg/ml, or no greater than 650 mg/ml, or no greater than 600 mg/ml, or no greater than 550 mg/ml, or no greater than 500 mg/ml, or no greater than 450 mg/ml, or no greater than 400 mg/ml. In the non-limiting extreme case, the relevant physiological fluid can be insoluble or practically insoluble in a strengthening excipient.

Typically, however, a relevant physiological fluid may be sparingly-soluble in a strengthening excipient. Thus, upon immersion of said strengthening excipient in said relevant physiological fluid, the stiffness (e.g., the elastic modulus) or the viscosity of said strengthening excipient may decrease somewhat compared with the stiffness or viscosity of the dry strengthening excipient. Similarly, upon immersing strengthening excipient in a relevant physiological fluid, the strain at fracture of said strengthening excipient may increase compared with the strain at fracture of the dry strengthening excipient. Because the strengthening excipient can be a solid, viscoelastic, semi-solid, or highly viscous material even after prolonged immersion in a relevant physiological fluid, it may also referred to herein as “stabilizing excipient”, or “viscoelastic excipient”. It may be further be noted that the terms “strengthening”, “mechanically strengthening”, “strength-enhancing”, and so on are generally used interchangeably herein.

In some embodiments of the invention herein, moreover, a mechanically strengthening phase (e.g., a “mechanically strengthening layer”, a “strength-enhancing layer”, “strengthening layer”, “strengthening phase”, and so on) may be attached to a core. In such embodiments, upon exposure to a relevant physiological fluid, the mechanical properties, such as elastic modulus, yield strength, tensile strength, viscosity, and so on, of said mechanically supported core (e.g., said core with attached strengthening phase) may generally be greater than the mechanical properties of said core without any mechanically strengthening phase. Typically, upon exposure to a relevant physiological fluid, at least a mechanical property, such as elastic modulus, yield strength, tensile strength, viscosity, and so on, of said mechanically supported core (e.g., said core with attached strengthening phase) may generally be at least two times greater than the corresponding mechanical property of said core without any strengthening phase. This includes, but is not limited to at least a mechanical property, such as elastic modulus, yield strength, tensile strength, viscosity, and so on, of said mechanically supported core (e.g., said core with attached strengthening phase) at least three, or at least four, or at least five or at least six, or at least seven, or at least eight times, or at least nine, or at least ten times greater than the corresponding mechanical property of said core without any strengthening phase.

In the invention herein, a material (e.g., a membrane, a layer, a strengthening phase, a composite mass, etc.) may generally be referred to as “viscoelastic” if it exhibits both viscous and elastic characteristics when undergoing deformation. By way of example but not by way of limitation, upon exposure of a viscoelastic material to a small stress or load for a short time, said viscoelastic material may behave similar to an elastic solid and spring back after unloading. If the viscous material is exposed to said small stress or load for a long time, however, said viscoelastic material may behave more like a highly viscous mass and deform plastically. An estimate of the “critical time” (e.g., the loading time below which a viscoelastic material may behave more like an elastic solid and above which said viscoelastic material may exhibit substantial plastic deformation) is the “relaxation time” defined as the ratio of elongational viscosity and elastic modulus of the material. Typically, as used herein the relaxation time of a viscoelastic material may be greater than about 0.1-0.5 seconds, and more preferably greater than about a second, and even more preferably greater than about 2-5 seconds. Also, upon loading and unloading a viscoelastic material the stress-strain curve of said viscoelastic material may exhibit a hysteresis loop. A non-limiting example of a viscoelastic material is rubber, such as natural rubber.

In the invention herein, moreover, a core may generally be referred to as “substantially encapsulated” by a surface layer if said surface layer covers (e.g., encloses, coats, etc.) at least 20 percent of the surface of said core. This includes, but is not limited to said surface layer covering at least 30 percent, or at least 40 percent, or at least 50 percent, or at least 60 percent, or at least 70 percent, or at least 80 percent, or at least 90 percent, or about 100 percent of the surface of said core.

In the invention herein, furthermore, the terms “semi-permeable layer”, “semi-permeable phase”, “semi-permeable membrane”, and so on may generally be understood as a membrane, layer, film, coating, coating film, etc. through which physiological fluid (e.g., water or water molecules) can fairly readily (e.g., fairly easily, fairly rapidly, etc.) pass upon exposure to said physiological fluid, but through which passage of at least an absorptive excipient is hindered or slow or slowed down. Thus, a “semi-permeable layer” is generally referred to as a membrane through which the diffusivity of physiological fluid (e.g., water) is substantially greater than the diffusivity of a fluid-absorptive excipient. Typically, upon exposure of a semi-permeable layer to a physiological fluid (e.g., water, saliva, gastric fluid, etc.) the diffusivity of said fluid through said layer is at least 5 times greater than the diffusivity of a fluid-absorptive excipient through said layer. This includes, but is not limited to diffusivity of physiological fluid through a semi-permeable layer at least 10 times, or at least 20 times, or at least 50 times, or at least 100 times greater than diffusivity of a fluid-absorptive excipient through said semi-permeable layer.

In the invention herein, drug release from a drug-containing solid (or a drug releasable solid, or a solid dosage form, or a pharmaceutical solid dosage form, etc.) refers to the conversion of drug (e.g., one or more drug particles, or drug molecules, or clusters thereof, etc.) that is/are embedded in or attached to the drug-containing solid (or a drug releasable solid, or a solid dosage form, or a pharmaceutical solid dosage form, etc.) to drug in a dissolution medium.

In the invention herein, the term “drug delivery” or “delivery” is generally referred to as “delivery of drug molecules to a human or animal body”. In specific circumstances it can refer to “delivery of drug molecules into the blood of a human or animal subject”.

In the invention herein, an “excipient matrix” may generally be understood as the component in a drug-containing solid that holds dispersed drug particles and/or dispersed drug molecules together.

As used herein, moreover, an excipient matrix may be understood “erodible” if said excipient matrix erodes or dissolves upon exposure to a relevant physiological fluid (e.g., gastric fluid) under physiological conditions.

Similarly, in the invention herein an excipient may be considered “soluble” if a solid particle of said excipient dissolves upon exposure to a relevant physiological fluid under physiological conditions (e.g., gastric fluid).

In the invention herein, moreover, a “stabilizing excipient” in an erodible excipient matrix may be understood as an excipient that slows down the erosion rate of said excipient matrix.

Further information related to the definition, characteristics, features, composition, analysis etc. of the disclosed dosage forms, and the elements for fabricating or constructing them, is provided throughout this specification.

### SCOPE OF THE INVENTION

It is contemplated that a particular feature described either individually or as part of an embodiment in this disclosure can be combined with other individually described features, or parts of other embodiments, even if the other features and embodiments make no mention of the particular feature. Thus, the invention herein extends to such specific combinations not already described. Furthermore, the drawings and embodiments of the invention herein have been presented as examples, and not as limitations. Thus, it is to be understood that the invention herein is not limited to these precise embodiments. Other embodiments apparent to those of ordinary skill in the art are within the scope of what is claimed.

By way of example but not byway of limitation, it is contemplated that compositions, articles, systems, devices, methods, and processes of the claimed invention encompass variations and adaptations developed using information from the embodiments described herein. Adaptation and/or modification of the compositions, systems, devices, methods, and processes described herein may be performed by those of ordinary skill in the relevant art.

Furthermore, where compositions, articles, and devices are described as having, including, or comprising specific components or features, or where processes and methods are described as having, including, or comprising specific steps, it is contemplated that, additionally, there are compositions, articles, and devices of the present invention that consist essentially of, or consist of, the recited components, and that there are processes and methods according to the present invention that consist essentially of, or consist of, the recited processing steps.

Similarly, where compositions, articles, and devices are described as having, including, or comprising specific compounds and/or materials, it is contemplated that, additionally, there are compositions, articles, and devices of the present invention that consist essentially of, or consist of, the recited compounds and/or materials.

It should be understood that the order of steps or order for performing certain action is immaterial so long as the invention remains operable. Moreover, two or more steps or actions may be conducted simultaneously.

The mention herein of any publication is not an admission that the publication serves as prior art with respect to any of the claims presented herein. Headers are provided for organizational purposes and are not meant to be limiting

### DETAILED DESCRIPTION OF THE INVENTION

### Aspects of the Dosage Form

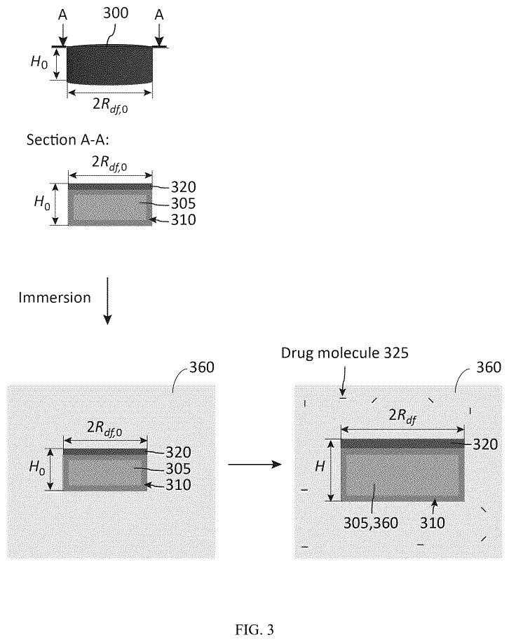

FIG. 3 presents a non-limiting example of a pharmaceutical solid dosage form 300 according to the invention herein. The dosage form 300 comprises a fluid-absorptive, expandable solid core 305, a mechanically strengthening semi-permeable layer 310, and a drug-containing solid 320 (e.g., a drug-containing layer, a drug-containing solid layer, a drug-containing surface layer, etc.). Said fluid-absorptive, expandable solid core 305 may be substantially encapsulated (e.g., substantially surrounded, substantially enclosed, substantially coated, etc.) by said mechanically strengthening semi-permeable layer 310. Additionally, at least a fraction (e.g., at least part, etc.) of said mechanically strengthening semi-permeable layer 310 may be disposed (e.g., positioned, located, placed, etc.) between said solid core 305 and said drug-containing solid 320.

Upon exposure of the dosage form 300 to a relevant physiological fluid 360 (e.g., gastric fluid) under physiological conditions (e.g., upon immersing the dosage form 300 in a relevant physiological fluid 360 under physiological conditions), the encapsulated solid core 305,310 may expand with fluid 360 absorption, and said drug-containing solid 320 may release drug 325 over time.

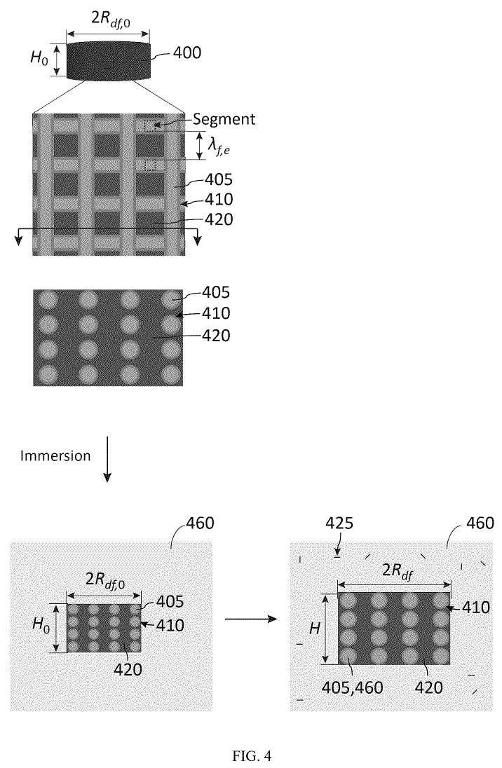

FIG. 4 presents another non-limiting example of a pharmaceutical solid dosage form 400 according to the invention herein. The dosage form 400 comprises a fluid-absorptive, expandable three dimensional structural framework of one or more fluid-absorptive structural elements 405 (e.g., an expandable solid core), a mechanically strengthening semi-permeable layer 410, and a drug-containing solid 420. Said structural framework of one or more elements 405 may comprise segments spaced apart from adjoining segments by element-free spacings, λ_{f,e}. Said structural framework 405 may further be substantially encapsulated (e.g., substantially surrounded, substantially coated, etc.) by said mechanically strengthening semi-permeable layer 410. Additionally, at least a fraction (e.g., at least part, etc.) of said mechanically strengthening semi-permeable layer 410 may be disposed (e.g., positioned, placed, etc.) between one or more elements or segments of said structural framework 405 and said drug-containing solid 420.

Upon exposure of the dosage form 400 to a relevant physiological fluid 460 (e.g., gastric fluid) under physiological conditions (e.g., upon immersing the dosage form 400 in a relevant physiological fluid 460 under physiological conditions), the encapsulated structural framework 405,410 may expand with fluid 460 absorption, and said drug-containing solid 420 may release drug 425 over time.

FIG. 4 illustrates a further non-limiting example of a pharmaceutical solid dosage form 400 according to the invention herein. The dosage form 400 comprises a fluid-absorptive, expandable three dimensional structural framework of criss-crossed stacked layers of one or more fluid-absorptive fibrous structural elements 405 (e.g., an expandable solid core), a mechanically strengthening semi-permeable layer 410, and a drug-containing solid 420. Said one or more fibrous structural elements 405 may comprise segments spaced apart from adjoining segments by element-free spacings, λ_{f,e}. Said structural framework 405 may further be substantially encapsulated (e.g., substantially surrounded, substantially coated, etc.) by said mechanically strengthening semi-permeable layer 410. Additionally, at least a fraction (e.g., at least part, etc.) of said mechanically strengthening semi-permeable layer 410 may be disposed (e.g., positioned, placed, etc.) between one or more elements or segments 405 thereof and said drug-containing solid 420.

Upon exposure of the dosage form 400 to a relevant physiological fluid 460 (e.g., gastric fluid) under physiological conditions (e.g., upon immersing the dosage form 400 in a relevant physiological fluid 460 under physiological conditions), the encapsulated structural framework 405,410 may expand with fluid 460 absorption, and said drug-containing solid 420 may release drug 425 over time.

FIG. 5 presents another non-limiting example of a pharmaceutical solid dosage form 500 according to the invention herein. The dosage form 500 comprises a three-dimensional structural framework of one or more fluid-absorptive elements 505, at least a mechanically strengthening semi-permeable layer 510, at least a drug-containing solid, and one or more free spaces (e.g., open free spaces, etc.). 520. Said structural framework of elements 505 may be substantially encapsulated (e.g., substantially surrounded, substantially coated, etc.) by said mechanically strengthening semi-permeable layer 510. Said encapsulated framework of elements 505,510 may comprise encapsulated segments spaced apart from adjoining encapsulated segments by encapsulated-element-free spacings, λ_{f,ee}, defining one or more encapsulated-element-free spaces in the pharmaceutical solid dosage form. At least one encapsulated-element-free space may have a free space (e.g., a channel, an open free space, an open channel, etc.) within or through it. Drug-containing solid may occupy encapsulated-element-free space (e.g., form an annulus, etc.) between a channel and the encapsulated framework of elements. Thus, generally, at least a fraction (e.g., at least part, etc.) of said mechanically strengthening semi-permeable layer 510 may be disposed (e.g., positioned, placed, etc.) between one or more elements or segments 505 thereof and said drug-containing solid 520.

FIG. 5 presents another non-limiting example of a pharmaceutical solid dosage form 500 according to the invention herein. The dosage form 500 comprises a three-dimensional structural framework of criss-crossed stacked layers of one or more fluid-absorptive fibrous structural elements 505, at least a mechanically strengthening semi-permeable layer 510, at least a drug-containing solid, and one or more free spaces 520 (e.g., open free spaces, etc.). Said structural framework 505 may be substantially encapsulated (e.g., substantially surrounded, substantially coated, etc.) by said mechanically strengthening semi-permeable layer 510. Said encapsulated framework of fibrous structural elements 505,510 may comprise encapsulated segments spaced apart from adjoining encapsulated segments by encapsulated-element-free spacings, λ_{f,ee}, defining one or more encapsulated-element-free spaces within the exterior volume of the encapsulated framework of fibrous elements 505,510. At least one encapsulated-element-free space may have free space (e.g., an open free space, a channel, an open channel, etc.) within or through it. Drug-containing solid may occupy encapsulated-element-free space (e.g., form an annulus, etc.) between a channel and the encapsulated framework of elements. Thus, generally, at least a fraction (e.g., at least part, etc.) of said mechanically strengthening semi-permeable layer 510 may be disposed (e.g., positioned, placed, etc.) between one or more elements or segments 505 thereof and said drug-containing solid 520.

It may be noted that in preferred embodiments, drug containing solid may also be attached to the encapsulated framework of one or more elements. Moreover, it may be noted that at least a three dimensional structural framework of one or more fluid-absorptive elements, at least a mechanically strengthening semi-permeable layer, at least a drug containing solid, and one or more free spaces or channels may form an expandable, drug releasable solid.

Another non-limiting example of a pharmaceutical solid dosage form according to the invention herein is shown schematically in FIG. 5. The dosage form 500 may comprise an expandable, drug releasable solid 501 comprising an outer surface 502 and an internal structure 504 contiguous with and terminating at said outer surface 502. Said internal structure 504 may comprise a fluid-absorptive, expandable three dimensional structural framework of one or more structural elements 505 (e.g., an expandable solid core), a mechanically strengthening semi-permeable layer 510, a drug-containing solid 520, and one or more free spaces 530. Said structural framework of one or more elements 505 may comprise segments spaced apart from adjoining segments by element-free spacings, λ_{f,e}. Said structural framework 505 may further be substantially encapsulated (e.g., substantially surrounded, substantially coated, etc.) by said mechanically strengthening semi-permeable layer 510. Said encapsulated structural framework of one or more elements 505,510 may comprise encapsulated segments spaced apart from adjoining encapsulated segments by encapsulated-element-free spacings, λ_{f,ee}, defining one or more encapsulated-element-free spaces within the expandable, drug releasable solid 501. At least part of said drug-containing solid 520 may be attached to said encapsulated structural framework 505,510 and occupy encapsulated-element-free space within the expandable, drug releasable solid 501. Said one or more encapsulated-element-free spaces within the expandable, drug releasable solid 501 may further comprise one or more free spaces or channels 530.

It may be noted that the role or function of one or more free spaces 530 in an expandable, drug releasable solid 501 can be to provide faster access of physiological fluid (e.g., gastric fluid) 560 to the interior of said expandable, drug releasable solid 501 after exposure of said expandable, drug releasable solid 501 to said physiological fluid 560. In some embodiments, therefore, one or more free spaces 530 (e.g., one or more interconnected free spaces, or one or more substantially interconnected free spaces, one or more substantially open free spaces, etc.) may be filled with a matter that is removable by a physiological fluid 560 under physiological conditions. Such matter that is removable by a physiological fluid under physiological conditions can, for example, be a gas which escapes a free space upon percolation of said free space by said physiological fluid. Such matter that is removable by a physiological fluid under physiological conditions can, however, also be a solid that is soluble or highly soluble in said physiological fluid, and thus may dissolve upon exposure to said physiological fluid.

Thus, upon exposure of the dosage form 500 or said expandable, drug releasable solid 501 to a relevant physiological fluid (e.g., gastric fluid) 560 under physiological conditions (e.g., upon immersing the dosage form 500 in a relevant physiological fluid 560 under physiological conditions), the fluid 560 may percolate one or more interconnected free spaces 530, and said encapsulated structural framework 505,510 may expand with fluid 560 absorption (e.g., due to diffusion or osmosis of physiological fluid into said framework, etc.). Also, said drug-containing solid 520 may release drug 525 over time.

FIG. 5 also shows a dosage form 500 comprising an expandable, drug releasable solid 501 comprising an outer surface 502 and an internal structure 504 contiguous with and terminating at said outer surface 502. Said internal structure 504 may comprise a fluid-absorptive, expandable three dimensional structural framework of one or more fibrous structural elements 505 (e.g., an expandable solid core), a mechanically strengthening semi-permeable layer 510, a drug-containing solid 520, and one or more free spaces 530. Said fibrous structural elements 505 may comprise segments spaced apart from adjoining segments by element-free spacings, λ_{f,e}. Said structural framework 505 may further be substantially encapsulated (e.g., substantially surrounded, substantially coated, etc.) by said mechanically strengthening semi-permeable layer 510. Additionally, at least a fraction (e.g., at least part, etc.) of said mechanically strengthening semi-permeable layer 510 may be disposed (e.g., positioned, placed, etc.) between one or more elements or segments 505 thereof and said drug-containing solid 520. In some embodiments, said one or more free spaces 530 may form at least a substantially interconnected (e.g., substantially connected, substantially open, etc.) free space 530 through the expandable, drug releasable solid 501.

Upon exposure of the dosage form 500 to a relevant physiological fluid 560 (e.g., gastric fluid) under physiological conditions (e.g., upon immersing the dosage form 500 in a relevant physiological fluid 560 under physiological conditions), the fluid 560 may percolate one or more free spaces 530, and said encapsulated structural framework 505,510 may expand with fluid 560 absorption. Also, said drug-containing solid 520 may release drug 525 over time.

It may be obvious to a person of ordinary skill in the art that many more non-limiting examples of dosage forms according to this invention could be given. Additional aspects and embodiments of dosage forms disclosed herein are described throughout this specification. Any more aspects and embodiments that would be obvious to a person of ordinary skill in the art are all within the spirit and scope of this invention.

### General Embodiments of the Dosage Form

Generally, said expandable solid core, or fluid-absorptive, expandable three dimensional structural framework, or element, or fiber, etc. 305,405,505 may comprise one or more first excipients. Said one or more first excipients may include at least a fluid-absorptive excipient. A non-limiting example of a fluid-absorptive first excipient is hydroxypropyl methylcellulose, and preferably hydroxypropyl methylcellulose with a molecular weight greater than 30 kg/mol, or even more preferably greater than 50 kg/mol.

Similarly, said mechanically strengthening semi-permeable layer 310,410,510 may comprise one or more second excipients. Said one or more second excipients may include at least a strengthening excipient. A non-limiting example of a strengthening excipient is methacrylic acid-ethyl acrylate copolymer, and preferably methacrylic acid-ethyl acrylate copolymer with a molecular weight of about 250 kg/mol (also referred to herein as “Eudragit L100-55”).

The drug-containing solid 320,420,520 may comprise at least one active pharmaceutical ingredient or drug. In some embodiments, the drug-containing solid also comprises one or more third excipients.

In preferred embodiments, moreover, upon exposure of the dosage form 300,400,500 to a physiological fluid (e.g., gastric fluid) 360,460,560, the encapsulated solid core 305,310,405,410,505,510 expands primarily with fluid 360,460,560 absorption. In the invention herein, a solid is generally understood as “expanding primarily with fluid absorption” if upon exposure of said solid to a physiological fluid, the greatest expansion of said solid (e.g., the greatest longitudinal expansion, such as the greatest increase in a length or normalized length; the greatest volumetric expansion, such as the greatest increase in a volume or normalized volume; etc.) is mostly or primarily due to the absorption of said physiological fluid 360,460,560. It may be noted that the encapsulated solid core 305,310,405,410,505,510 may generally transition to a viscoelastic mass, viscoelastic solid, viscoelastic composite, semi-solid, or viscous (e.g., highly viscous) mass as it expands with fluid 360,460,560 absorption.

In preferred embodiments, upon exposure of the dosage form 300,400,500 to a relevant physiological fluid (e.g., gastric fluid) 360,460,560 under physiological conditions, a mechanically strengthening, semi-permeable layer 310,410,510 forms a semi-permeable, viscoelastic membrane.

In preferred embodiments, moreover, upon exposure of the dosage form 300,400,500 to a relevant physiological fluid (e.g., gastric fluid) 360,460,560 under physiological conditions, a mechanically strengthening, semi-permeable layer 310,410,510 is substantially permeable to said physiological fluid 360,460,560. In the invention herein, a membrane or layer is generally referred to as “substantially permeable” to a physiological fluid 360,460,560 if the diffusivity of water in said membrane or layer is greater than about 0.0001 times the self-diffusivity of water. Thus, generally, in the invention herein a membrane or layer is understood “substantially permeable” to a physiological fluid 360,460,560 if the diffusivity of water in said membrane or layer under physiological conditions (e.g., at a temperature of 37° C.) is greater than about 1×10⁻¹³ m²/s.

Similarly, in preferred embodiments, upon exposure of the dosage form 300,400,500 to a physiological fluid (e.g., gastric fluid) 360,460,560, said mechanically strengthening, semi-permeable layer 310,410,510 is substantially impermeable to at least one fluid-absorptive first excipient. In the invention herein, a membrane or layer 310,410,510 is generally understood “substantially impermeable” to a fluid-absorptive first excipient if a diffusivity of said fluid-absorptive first excipient in or through said membrane or layer 310,410,510 is smaller than 0.1 times the diffusivity of water in or through said membrane or layer 310,410,510. This includes, but is not limited to a diffusivity of said fluid-absorptive first excipient in or through said membrane or layer 310,410,510 smaller than 0.05 times, or smaller than 0.02 times, or smaller than 0.01 times, or smaller than 0.005 times the diffusivity of water in or through said membrane or layer 310,410,510.

In preferred embodiments, moreover, upon exposure of the dosage form 300,400,500 to a relevant physiological fluid (e.g., gastric fluid) 360,460,560 under physiological conditions, the mechanically strengthening semi-permeable layer 310,410,510 expands due to an internal pressure in the core 305,405,505 said internal pressure generated by osmotic flow of fluid 360,460,560 into said core 305,405,505.

Generally, furthermore, upon exposure of the dosage form 300,400,500 to a relevant physiological fluid (e.g., gastric fluid) 360,460,560, the encapsulated solid core 305,310,405,410,505,510 forms an expanded, viscoelastic composite mass 305,310,360,405,410,460,505,510,560.

In preferred embodiments, moreover an expandable solid core 305,405,505 generally has at least one exterior dimension (e.g., a length, width, or thickness) greater than 3 mm.

Additional embodiments of dosage forms disclosed herein are described throughout this specification. Any more embodiments that would be obvious to a person of ordinary skill in the art are all within the spirit and scope of this invention.

### Theoretical Models of Dosage Form Properties

This section presents non-limiting ways by which the properties of disclosed gastroretentive dosage forms may be modeled. For comparison, models of the properties of an immediate-release particulate dosage form are also presented. The models and examples will enable one of skill in the art to more readily understand the conceptual details and advantages of the invention. The models and examples are for illustrative purposes only, and are not meant to be limiting in any way.

### Theoretical Models of Dosage Form Properties: Microstructures and Compositions of Dosage Forms

### (a) Microstructure of Particulate Dosage Form

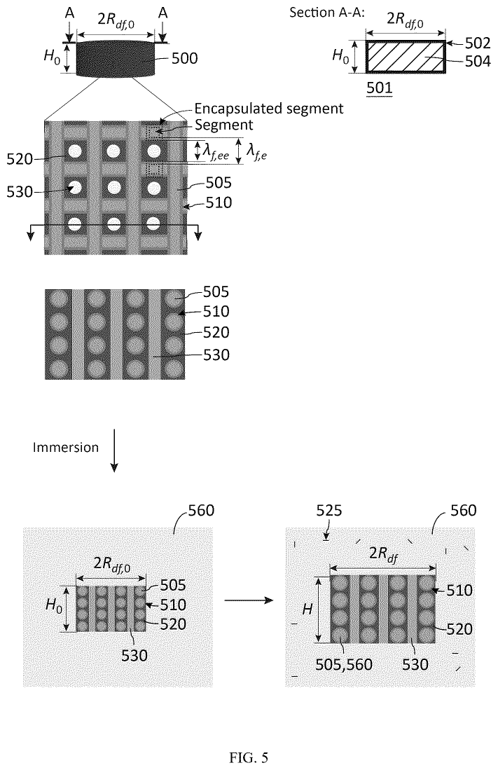

As shown schematically in FIG. 6, the particulate dosage form 600 considered in the non-limiting models presented herein may comprise an immediate-release gelatin capsule 603 filled with loose particles of drug 624 and various particles of excipient 644. The non-limiting model drug 624 may be nilotinib hydrochloride monohydrate. The amount of drug 624 in the capsule 703 may be 200 mg, and the radius of the drug particles 624 may be about 18.5 μm, Table 1.

### (b) Microstructure of Gastroretentive Dosage Form

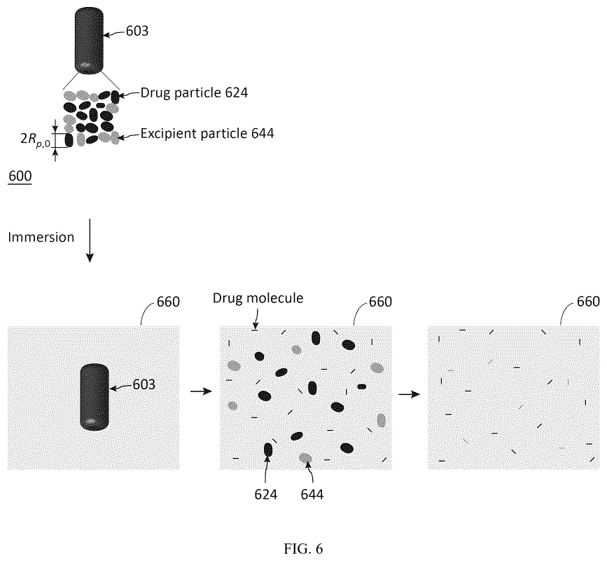

The microstructure of the non-limiting gastroretentive dosage form considered in the models is schematically illustrated in FIG. 7. The dosage form 700 comprises an expandable, drug releasable solid 701 comprising an outer surface 702 and an internal structure 704 contiguous with and terminating at said outer surface 702.

Said internal structure comprises 704 at least an expandable, three dimensional structural framework of criss-crossed stacked layers of one or more fluid-absorptive fibers 705, a mechanically strengthening semi-permeable layer 710, a drug-containing solid 720, and one or more free spaces or channels 730.

Said structural framework 705 may be substantially encapsulated by said mechanically strengthening layer 710. Said encapsulated structural framework 705 may comprise encapsulated segments spaced apart from adjoining encapsulated segments by encapsulated-fiber-free spacings, λ_{f,ee}, defining one or more encapsulated-fiber-free spaces within the expandable, drug releasable solid 701. At least one encapsulated-fiber-free space may have an open channel 730 through it.

Drug-containing solid 720 may be attached to the encapsulated framework of fibers 705,710 and occupy encapsulated-fiber-free space (e.g., form an annulus) between an open channel 730 and the encapsulated framework of fibers 705,710.

It may be noted that a “three-dimensional structural framework of criss-crossed stacked layers of one or more fluid-absorptive fibers” is also referred to herein as a “three-dimensional structural framework of one or more fluid-absorptive fibers stacked in a cross-ply arrangement” or as a “three-dimensional structural framework of one or more fluid-absorptive fibers forming fiber layers stacked in a cross-ply arrangement”. The non-limiting dosage form modeled herein (and included in this invention) further comprises an ordered or substantially ordered structure; the one or more fibers in the criss-crossed stacked layers of one or more fibers may be orderly or substantially orderly arranged. Moreover, because the gastroretentive dosage form modeled herein is based on a framework of fibers, it is also referred to as “fibrous dosage form”. Selected microstructural parameters of the specific non-limiting fibrous dosage form modeled herein are listed in Table 1.

Any other ways of describing the structure of the non-limiting fibrous dosage forms modeled herein are all within the spirit and scope of this invention.

### (c) Composition of Gastroretentive Dosage Form

The composition, or formulation, of the various phases or “structural phases” or “structural components” of the non-limiting gastroretentive dosage form 700 considered in the models may be as shown schematically in the inset of FIG. 7.

The structural framework of fluid-absorptive fibers 705 may comprise a mixture of a physiological fluid-absorptive excipient 715, a mechanically strengthening excipient 716, and a gastrointestinal contrast agent. The physiological fluid-absorptive excipient 715 in the fibers 705 may comprise hydroxypropyl methylcellulose (HPMC) with large molecular weight, referred to herein as “HPMC2”. The mechanically

TABLE 1
Selected microstructural parameters, composition, and properties of the
non-limiting particulate and fibrous dosage forms modeled in this work.

| | | |
|---|---|---|
| Symbol | Description | Value |

Particulate

| | | | |
|---|---|---|---|
| M_{d,0} | drug mass in dosage form | 200 | mg |
| R_{p,0} | radius of drug particles in dosage form | 18.5 | μm |

Fibrous

| H_{c,0} | channel length in solid dosage form | 8 | mm |
|---|---|---|---|
| | (thickness of solid dosage form) | | |
| h_{a,0} | thickness of drug-loaded, solid annulus | 156 | μm |
| M_{d,0} | drug mass in dosage form | 200 | mg |
| nₐ or n_{c} | number of annuli, or channels, in dosage form | 91.4^{a} | |
| R_{c,0} | radius of inner channel of drug-loaded annulus | 302 | μm |
| R_{df,0} | radius of dosage form | 7 | mm |
| R_{f,0} | fiber radius | 165 | μm |
| w_{d,a} | weight fraction of drug (nilotinib) in annulus | 0.6 | |
| w_{HPMC1,a} | weight fraction of low-molecular-weight HPMC in annulus | 0.36 | |
| w_{ee,a} | weight fraction of enteric excipient (Eudragit L100-55) in annulus ^{b} | 0.04 | |
| λ₀ | inter-fiber spacing | 1298 | μm |
| ρₐ | density of drug-loaded annulus | 1302 | kg/m^{3c} |

| | | |
|---|---|---|
| φ_{ec} | volume fraction of enteric excipient coating in the dosage form | 0.15 |
| φ_{f} | volume fraction of solid fiber core in dosage form | 0.30 |
| φ_{HPMC2,f} | volume fraction of high-molecular-weight HPMC in solid fiber core | 0.46 |

All parameters are the same as those of the experimental dosage forms presented in section ″Experimental Examples″.
^{a}Calculated as nₐ = n_{c} = πR_{df,0}²/λ₀².
^{b}The trade name of the non-limiting enteric methacrylic acid-ethyl acrylate excipient used is Eudragit L100-55.
^{c}Calculated as ρₐ = 1/(w_{HPMC1,a}/ρ_{HPMC1} + w_{d,a}/ρ_{d}+ w_{ee,a}/ρₑₑ), where the densities of HPMC1, nilotinib, and enteric excipient, ρ_{HPMC1} = 1300, ρ_{d} = 1360, and ρₑₑ = 800 kg/m³.

strengthening excipient 716 in the fibers 705 may comprise methacrylic acid-ethyl acrylate copolymer (e.g., Eudragit L100-55). The gastrointestinal contrast agent in the fibers 705 may comprise barium sulfate (not shown in the schematic; it may not be needed to produce a dosage form that is functional). The weight fractions of HPMC2 715, Eudragit L100-55 716, and barium sulfate in the solid fibers 705 may be about 0.4375, 0.2625, and 0.3, respectively.

The mechanically strengthening, semi-permeable layer 710 (e.g., a mechanically strengthening phase) may comprise a mechanically strengthening excipient 717. The mechanically strengthening excipient 717 in the strengthening phase 710 may comprise methacrylic acid-ethyl acrylate copolymer (e.g., Eudragit L100-55).

The drug-containing solid 720 may comprise a drug 721,722 (e.g., in the form of drug particles 721 and/or drug molecules 722) and one or more excipients 825,826 for releasing the drug at the desired rate after immersing the dosage form 700 in a physiological fluid under physiological conditions. The drug 721,722 may comprise nilotinib (e.g., nilotinib hydrochloride monohydrate). The one or more excipients 725,726 may include at least an excipient 725 that is soluble in a physiological fluid (e.g., gastric fluid, simulated gastric fluid, water, etc.) and a mechanically strengthening excipient 726. The physiological fluid-soluble excipient 725 in the drug-containing solid 720 may comprise hydroxypropyl methylcellulose (HPMC) with low molecular weight, referred to herein as “HPMC1”. The mechanically strengthening excipient 726 in the drug-containing solid 720 may comprise methacrylic acid-ethyl acrylate copolymer (e.g., Eudragit L100-55). The weight fractions of nilotinib 721,722, HPMC1 725, and Eudragit L100-55 726 in the drug-containing solid 720 may be about 0.6, 0.36, and 0.04, respectively.

The one or more open channels 730 may be filled with air.

Any other ways of describing the composition of the non-limiting fibrous dosage forms modeled herein are all within the spirit and scope of this invention.

### Theoretical Models of Dosage Form Properties: In Vitro Expansion, Mechanical Properties, and Drug Release

### (a) In Vitro Drug Release by Particulate Dosage Form

Upon immersing the particle-filled capsule in a dissolution fluid (e.g., acidic water, simulated gastric fluid, gastric fluid, etc.), the capsule may dissolve and drug particles may be released. An analysis of the dissolution of the gelatin capsule is beyond the scope of this disclosure. Suffice it to state, however, that a typical immediate-release gelatin capsule may dissolve in 3-6 minutes, and thereafter release the drug particles almost immediately into the dissolution fluid. The drug particles in the dissolution fluid may then dissolve and release drug molecules.

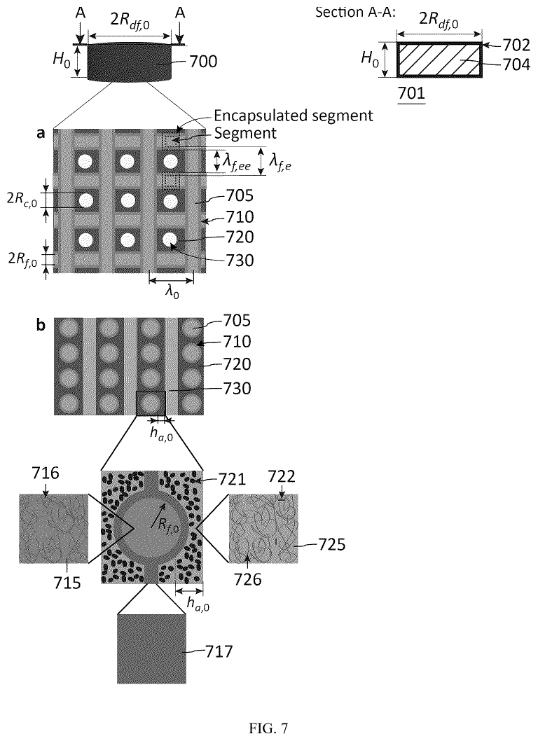

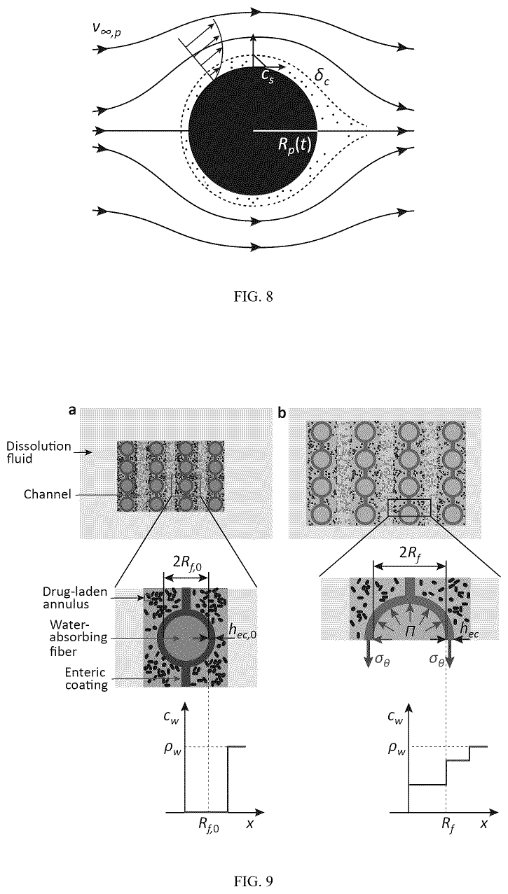

As shown in FIG. 8, for estimating the dissolution time of the drug particles, a spherical drug particle of radius, Rₚ, exposed to free-flowing dissolution fluid with far-field velocity, v_{∞,p}, may be considered. From companion work (see. e.g., A. H. Blaesi and N. Saka, Gastroretentive fibrous dosage forms for prolonged delivery of sparingly soluble tyrosine kinase inhibitors. Part 1: Dosage form design, and models of expansion, post-expansion mechanical strength, and drug release, to be published in the International Journal of Pharmaceutics, and referred to herein as “REF. [1]”), if the volume of dissolution fluid is large and the drug concentration in the fluid, c_{d}(t)˜0, the dissolution time of the particle may be estimated as:

$${t}_{dis} - {t}_{d , c} = 0.94 ( \frac{{ρ}_{d}}{{c}_{s}} ) {( \frac{{R}_{p , 0}^{5}}{{D}_{d}^{2} {v}_{∞ , p}} )}^{1 / 3} \qquad ( 1 )$$

where t_{dis} and t_{d,c} are the times at which the particle and the capsule have dissolved after immersing the dosage form in a dissolution fluid, ρ_{d} is the density of the drug particle, cₛ the drug solubility in the dissolution fluid, R_{p,0} the initial radius of the particle, D_{d} the drug diffusivity in the dissolution fluid, and v_{∞,p} the far-field velocity of the dissolution fluid.

Substituting the non-limiting parameter values ρ_{d}=1360 kg/m³, cₛ=1 mg/ml, R_{p,0}=18.5 μm, D_{d}=4.22×10⁻¹⁰ m²/s, and v_{∞,p}=29 μm/s in Eq. (1), t_{dis}−t_{d,c}=16 min. Thus, the drug particle may be dissolved just about 16 minutes after dissolution of the capsule.

Similarly, from companion work (see, e.g., REF. [1]), the drug release rate by a collection of identical drug particles that do not interact with each other may be estimated as:

$$\frac{{dm}_{d , r}}{dt} = 1.92 {M}_{d} ( \frac{{c}_{s} - {c}_{d} ( t )}{{ρ}_{d}} ) {( \frac{{D}_{d}^{2} {v}_{∞ , p}}{{R}_{p}^{5}} )}^{1 / 3} {t}_{d , c} \leq t \leq {t}_{dis} \qquad ( 2 )$$

where M_{d} is the mass of drug particles in the dissolution fluid, c_{d}(t) the drug-molecule concentration in the dissolution fluid, and Rₚ the radius of the drug particles.

Eq. (2) stipulates that the drug release rate may be time-dependent because both the drug mass in the dissolution fluid and the particle radius may be time-dependent. Regardless, substituting the non-limiting initial values, M_{d,0}=200 mg and R_{p,0}=18.5 μm, and the non-limiting parameters cₛ=1 mg/ml, ρ_{d}=1360 kg/m³, D_{d}=4.22×10⁻¹⁰ m²/s, and v_{∞,p}=29 μm/s, in Eq. (2), the “initial” drug release rate in a large volume of dissolution fluid with c_{d}(t)˜0 is 23 mg/min. At this release rate, 200 mg of drug particles dissolve in 8.7 minutes, just 1.8 times shorter than the dissolution time calculated by Eq. (1) above. Thus, in a large volume of dissolution fluid the “initial” drug release rate may be considered a fairly reasonable approximation of the release rate by the particles.

Any other models for estimating the dissolution time and/or the drug release rate by particulate dosage forms or drug particles obvious to a person of ordinary skill in the art are all within the spirit and scope of this disclosure.

### (b) In Vitro Expansion of Gastroretentive Fibrous Dosage Form

By contrast, upon immersing the fibrous dosage forms in a dissolution fluid (e.g., acidic water, simulated gastric fluid, gastric fluid, etc.), the fluid may rapidly percolate the open channels, and diffuse into the solid walls comprising the drug-excipient annulus, the enteric fiber coating, and the high-molecular weight HPMC (HPMC2) fibers, FIGS. 9a and 9b. The enteric coating over the fibers may be considered a semi-permeable membrane. It may allow the passage of small water molecules into the fiber, but block the passage of the large HPMC2 molecules out. Thus, as water diffuses into the fibers an internal pressure may develop in the fiber. The internal pressure may induce a tensile stress in the coating, and if the coating is deformable, the coating, fiber, and dosage form may expand, FIG. 9b.

An exact analysis of the coupled diffusion-expansion problem is beyond the scope of this disclosure. From companion work (see, e.g., REF. [1]), the normalized radial expansion of the dosage form may be estimated as:

$$\begin{matrix}\frac{Δ {R}_{df} ( t )}{{R}_{df , 0}} = {k}_{2} \frac{{σ}_{θ} t}{η} = {k}_{2} {\prod }_{0} \frac{2 {φ}_{f}}{{φ}_{ec}} \frac{t}{η} \\ = {k}_{2} RT \frac{{φ}_{HPMC 2 , f} {ρ}_{HPMC 2}}{{M}_{HPMC 2}} \frac{2 {φ}_{f}}{{φ}_{ec}} \frac{t}{η} = {C}_{2} t\end{matrix} \qquad ( 3 )$$

where ΔR_{df}=R_{df}−R_{df,0}, and R_{df} is the radius of the expanding dosage form, R_{df,0} the radius of the initial solid dosage form, k₂ a dimensionless constant, σ_{θ} the tensile stress in the coating, η the elongational viscosity of the acidic water-soaked coating, Π₀ the osmotic pressure in the fiber initially (e.g., at time, t=0 and at “zero” expansion), φ_{f} and φ_{ec}, respectively, are the volume fractions of fiber and coating in the solid dosage form, R is the ideal gas constant, T the absolute temperature, φ_{HPMC2,f} the volume fraction of HPMC2 in the fibers, and ρ_{HPMC2} the solid density of HPMC2, M_{HPMC2} the molecular weight of HPMC2, and C₂ a dimensional constant.

It will be shown later in subsection “Example 6: In vitro expansion of gastroretentive dosage forms” of section “Experimental Examples” that a non-limiting approximation of the constant, k₂˜0.108. Substituting this value and the non-limiting parameter values, R=8.314 J/molK, T=310 K, φ_{HPMC2,f}=0.46, ρ_{HPMC2}=1300 kg/m³, M_{HPMC2}=120 kg/mol, φ_{f}=0.3, φ_{ec}=0.15, and η=1.36×10⁸ Pa·s in Eq. (3), the dosage form may expand to about 1.5 times the initial radius in about 3.5 hours. Prior work suggests that this amount of expansion may prevent the premature passage of the dosage form through the pylorus (see, e.g., A. H. Blaesi, D. Kümmerlen, H. Richter, N. Saka, Mechanical strength and gastric residence time of expandable fibrous dosage forms, Int. J. Pharm. 613 (2022) 120792).

Any other models for estimating the expansion rate of the disclosed dosage forms obvious to a person of ordinary skill in the art are all within the spirit and scope of this disclosure.

### (c) Mechanical Strength of Expanded Fibrous Dosage Form

In the stomach, the expanded dosage form may be exposed to repeated compression-decompression pulses by the stomach walls. Thus, to remain in the stomach for prolonged time, the expanded dosage form may withstand these loads.

From companion work (see, e.g., REF. [1]), a predominant component that may add strength to the expanded dosage form is the strengthening coating over the fibers. If the width of the compression-decompression pulses, τₚᵤₗₛₑ˜1 s, is much smaller than relaxation time of the acidic water-soaked coating, τᵣₑₗ˜24 s, the coating, and the expanded dosage form, may behave essentially like an elastic solid under these loads, and may fracture.

Treating the internally pressurized coating network in the expanded dosage form as a cellular solid, the fracture strength of the expanded dosage form may be estimated as (see, e.g., REF. [1]):

$${σ}_{f , df} = {C}_{8} {φ}_{ec}^{n} {σ}_{ec} \qquad ( 4 )$$

where C₈ is a constant, φ_{ec} the volume fraction of the enteric coating in the expanded dosage form, n a constant, and σ_{ec} the fracture strength of the acidic water-soaked coating.

Substituting the non-limiting values, C₈=0.93, φ_{ec}=0.15, n=1.19, and σ_{ec}=1.8 MPa in Eq. (4), the fracture strength, σ_{f,df}=0.175 MPa. Prior work suggests that this fracture stress may be sufficient to hold the dosage form in the stomach for prolonged time (see, e.g., A. H. Blaesi, D. Kümmerlen, H. Richter, N. Saka, Mechanical strength and gastric residence time of expandable fibrous dosage forms, Int. J. Pharm. 613 (2022) 120792). Thus, with the coating volume fraction, φ_{c}=0.15, both the expansion rate and the post-expansion mechanical strength of the dosage form may be fairly adequate.

Any other models for estimating the post-expansion mechanical properties of the disclosed dosage forms obvious to a person of ordinary skill in the art are all within the spirit and scope of this disclosure.

### (d) In Vitro Drug Release by Gastroretentive Fibrous Dosage Form

In the dissolution fluid (e.g., acidic water, simulated gastric fluid, gastric fluid, etc.), moreover, the gastroretentive fibrous dosage form may release drug from the drug-containing annuli into the dissolution fluid.

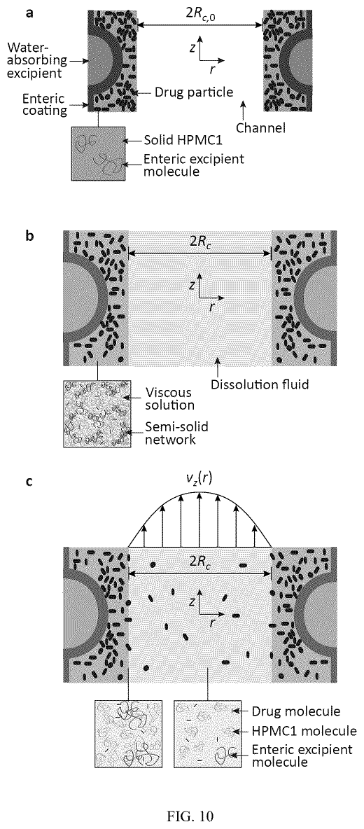

As shown in the non-limiting FIG. 10a, initially the solid annuli in the dosage form may comprise drug particles dispersed in a solid solution of HPMC1 and enteric excipient. Upon immersing in the dissolution fluid, the water may percolate the open channels, and diffuse into the excipient matrix, FIG. 10b. The HPMC1 molecules in the matrix may be mutually soluble with acidic water, but the enteric excipient molecules may not. Thus, as shown in the inset of the non-limiting FIG. 10b, as water diffuses in, the HPMC1 and enteric excipient molecules may separate out into two phases: a viscous solution of HPMC1 and water, and a stabilizing, semi-solid cellular network of enteric excipient.

As shown in the non-limiting FIG. 10c, at the surface of the annulus the HPMC1 molecules may diffuse into the channel and may be convected through the channel into the dissolution fluid outside. As a result, the excipient matrix may erode.

If the excipient in the annuli erodes or dissolves faster than the drug, the drug particles at the surface of the annuli may be released as the surrounding HPMC1-enteric excipient matrix dissolves. Further assuming that the excipient concentration at the channel exit is about the same as that at the interface between the annulus and the fluid-filled channel, FIG. 11, as detailed in a companion work (see, e.g., REF. [1]), the drug release rate may be estimated as:

$$\frac{{dm}_{d , r}}{dt} = ( \frac{{w}_{d , a}}{{w}_{HPMC 1 , a}} ) {n}_{c} π {R}_{c}^{2} 〈 {v}_{z} 〉 {c}_{HPMC 1}^{*} \qquad ( 5 )$$

where w_{d,a} and w_{HPMC1,a}, respectively, are the weight fractions of drug and HPMC1 in the solid annuli, n_{c} the number of channels in the dosage form, R_{c} the channel radius [symbol] v₂ [symbol] the average velocity of dissolution fluid through a channel, and c_{HPMC1}* the concentration of HPMC1 at the channel surface (i.e., the HPMC1 concentration at which the HPMC1-enteric excipient composite may disentangle).

Eq. (5) shows that the drug release rate may be constant and controllable by the number of channels, n_{c}, and the HPMC1 concentration at which the HPMC1-enteric excipient composite may disentangle, c_{HPMC1}*. For the non-limiting parameters, w_{d}=0.6, w_{HPMC1}=0.36, n_{c}=91.4, R_{c}=453 μm, [symbol] v_{z} [symbol] =6.68 μm/s, and c_{HPMC1}*=3.27 mg/ml, by Eq. (5), dm_{d,r}/dt=7.72 mg/h. This is about 179 times slower than the release rate of drug particles estimated in subsection (a) above.

Moreover, by integrating the drug release rate over time the mass of drug released may be obtained as:

$${m}_{d , r} ( t ) = ( \frac{{w}_{d , a}}{{w}_{HPMC 1 , a}} ) {n}_{c} π {R}_{c}^{2} 〈 {v}_{z} 〉 {c}_{HPMC 1}^{*} t \qquad ( 6 )$$

Dividing m_{d,r}(t) by the drug mass in the dosage form and rearranging may give the fraction of drug released as:

$$\frac{{m}_{d , r} ( t )}{{M}_{d , 0}} = π {n}_{c} ( \frac{{w}_{d , a}}{{w}_{HPMC 1 , a}} ) ( \frac{{R}_{c}^{2} 〈 {v}_{z} 〉 {c}_{HPMC 1}^{*} t}{{M}_{d , 0}} ) \qquad ( 7 )$$

Thus, under the assumptions made, the fraction of drug released may increase fairly linearly with time until it may reach unity at the dissolution time, t_{dis}.

By substituting m_{d,r}/M_{d,0}=1 into Eq. (7) and rearranging, the drug dissolution time may be estimated as:

$${t}_{dis} = \frac{1}{π {n}_{c}} ( \frac{{w}_{HPMC 1 , a}}{{w}_{d , a}} ) ( \frac{{M}_{d , 0}}{{R}_{c}^{2} 〈 {v}_{z} 〉 {c}_{HPMC 1}^{*}} ) \qquad ( 8 )$$

For the non-limiting parameter values listed in Table 1, and [symbol] v_{z} [symbol] =6.68 μm/s and c_{HPMC1}*=3.27 mg/ml, by Eq. (8) t_{dis}=26 hours.

[symbol]

The calculated dissolution time is of the order of the reasonable dosing interval of a day. Thus, the gastroretentive fibrous dosage forms may enable a substantially constant drug release rate for prolonged time.

Any other models for estimating the drug release rate of the disclosed dosage forms obvious to a person of ordinary skill in the art are all within the spirit and scope of this disclosure.

### Theoretical Models of Dosage Form Properties: Gastric Residence Time and Drug Concentration in Blood

Upon administering to a human or animal, however, the course of the dosage form and of the drug may generally be far more complex than that in an in vitro dissolution vessel.

(a) Overview of Drug Release, Absorption, Distribution, and Elimination after Administering a Dosage Form to a Human or Animal

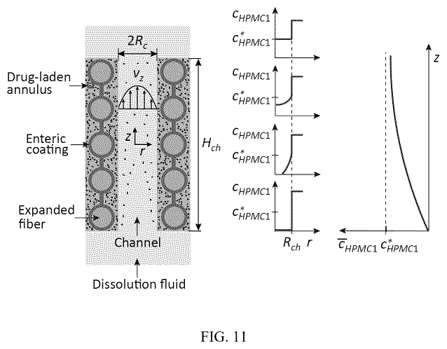

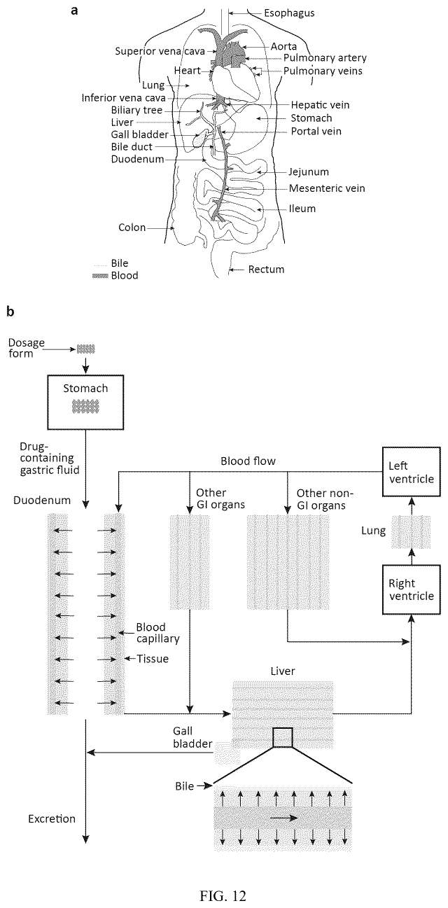

FIG. 12a presents a non-limiting schematic of some of the relevant organs of the human body for in vivo drug release, absorption, distribution, and elimination. FIG. 12b is a flow diagram of a non-limiting passage of the drug through the body. The relevant biophysical processes may be as follows:
Upon oral ingestion, the dosage form may pass into the stomach and release drug molecules.
Drug molecules released in the stomach may pass into the duodenum with the gastric fluid flow, and diffuse into the duodenal blood capillaries.
The blood in the duodenal capillaries may then join with that in other gastrointestinal organs and drain into the portal vein and the liver.
In the liver, some of the drug may be eliminated from the blood by diffusion into the bile.
The remaining drug may exit the liver with the blood flow, combine with the venous blood of all other organs, and be supplied to the right ventricle of the heart.
The right ventricle may pump the blood into the lung capillaries and the left ventricle.
The left ventricle may then pump the blood back into the blood capillaries of the duodenum and all other organs.
Within all organs, drug may diffuse from the blood capillaries into the tissue (cellular and extracellular space without blood) and back.

In what follows, the above processes are modeled for estimating drug concentration in blood versus time after administering particulate and fibrous dosage forms. In all models the anatomical and physiological parameter values of the fasted dog may be used. The models may, however, be readily extended to humans.

### (b) Gastric Residence Time of Particulate Dosage Form

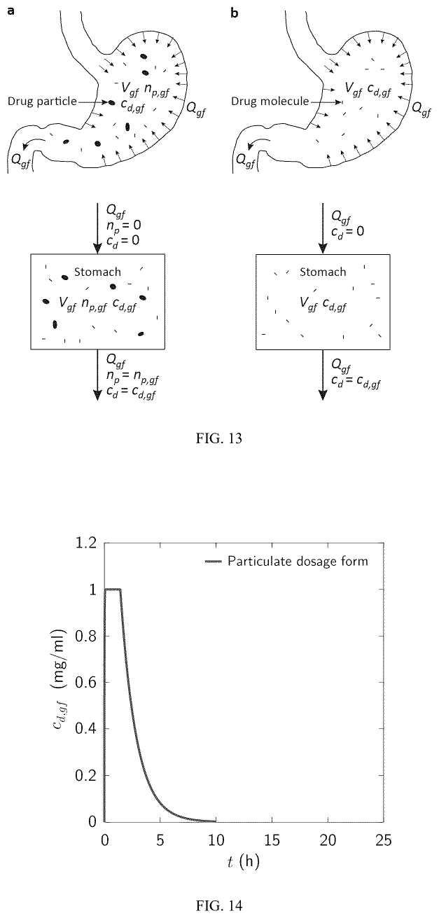

Upon entering the stomach, the particle-filled capsule may disintegrate and release drug particles into the gastric fluid which may then dissolve. In the present models, the mass of drug per unit volume of the gastric fluid, M_{d,0}/V_{gf}, may generally be far greater than the solubility of the drug, c_{s,gf}. Thus, as shown schematically in the non-limiting FIG. 13a, the drug particles may not dissolve entirely; part of the particles may flow out of the stomach with the gastric fluid.

Assuming that (a) the volumetric inflow and outflow rates of gastric fluid, Q_{gf}, are the same and time-invariant, (b) the particles in the stomach are perfectly mixed with the gastric fluid, and (c) no drug particle will dissolve completely, the rate at which the number of drug particles per unit volume of gastric fluid, n_{p,gf}, changes may be written as:

$${V}_{gf} \frac{{dn}_{p , gf}}{dt} = - {Q}_{gf} {n}_{p , gf} \qquad ( 9 a )$$

where V_{gf} is the volume of the gastric fluid.

If the drug particles are released immediately after the capsule reaches the stomach, at time, t=0 the number of drug particles in the gastric fluid may be written as:

$$\begin{matrix}{n}_{p , gf} = {n}_{p , 0} & t = 0\end{matrix} \qquad ( 9 b )$$

where n_{p,0} is the number of drug particles in the solid capsule per unit volume of gastric fluid.

Rearranging and integrating Eq. (9a) with the initial condition, Eq. (9b), may give:

$${n}_{p , gf} = {n}_{p , 0} exp ( - \frac{{Q}_{gf}}{{V}_{gf}} t ) \qquad ( 10 )$$

From Eq. (10) the number of drug particles in the gastric fluid may decrease to 37 percent of the initial value at the characteristic residence time:

$${t}_{r , p} = \frac{{V}_{gf}}{{Q}_{gf}} \qquad ( 11 )$$

For the non-limiting parameter values roughly resembling an empty stomach of a dog, V_{gf}=20 ml and Q_{gf}=14 ml/h, by Eq. (11) t_{r,p}˜1.43 h, Table 2 later.

For further details related to the estimation of the gastric residence time of drug particles, see, e.g., A. H. Blaesi and N. Saka, Gastroretentive fibrous dosage forms for prolonged delivery of sparingly soluble tyrosine kinase inhibitors. Part 3: Theoretical models of drug concentration in blood, to be published in the International Journal of Pharmaceutics, and referred to herein as “REF. [3]”.

Any other models for estimating the gastric residence time of particulate dosage forms or drug particles obvious to a person of ordinary skill in the art are all within the spirit and scope of this disclosure.

(c) Drug Concentration in Gastric Fluid after Administering Particulate Dosage Form

Generally, the drug in the gastrointestinal system should be in molecular form for absorption by blood. Indeed, as was shown schematically in FIG. 13a, as the drug particles may dissolve in the stomach, the molecular concentration of drug in the gastric fluid may increase.

Because the particles release drug rapidly, and in the present models, the mass of drug per unit volume of the gastric fluid, M_{d,0}/V_{gf}>>c_{s,gf}, the drug solubility in gastric fluid, the drug concentration in gastric fluid may rapidly rise to solubility. Thus, up to the “gastric residence time” of drug particles, t_{r,p}, the drug concentration in gastric fluid may roughly be approximated as:

$$\begin{matrix}{c}_{d , gf} = {c}_{s , gf} & t \leq {t}_{r , p}\end{matrix} \qquad ( 12 )$$

An estimate of the solubility of nilotinib in an empty stomach of a dog, c_{s,gf}˜1 mg/ml.

After t_{r,p} the stomach may be assumed to be mostly depleted of drug particles, and only comprise residual drug molecules, FIG. 13b. From a companion work (see. e.g., REF. [3]), the residual drug molecules may flow out of the stomach with the gastric fluid flow. A rough estimate of the drug concentration in gastric fluid may be written as:

$$\begin{matrix}{c}_{d , gf} = {c}_{s . gf} exp ( - \frac{{Q}_{gf} ( t - {t}_{r , p} )}{{V}_{gf}} ) & t > {t}_{r , p}\end{matrix} \qquad ( 13 )$$

Thus, after the “residence time” of the particles, the concentration of drug molecules in the gastric fluid may drop exponentially at the time constant, τ_{gf}=V_{gf}/Q_{gf}. For the non-limiting parameter values roughly resembling an empty stomach of a dog, V_{gf}=20 ml and Q_{gf}=14 ml/h, the time constant, τ_{gf}˜1.43 h.

FIG. 14 plots the drug concentration in the gastric fluid by Eqs. (12) and (13) for the non-limiting parameters, c_{s,gf}=1 mg/ml, V_{gf}=20 ml, Q_{gf}=14 ml/h, and t_{r,p}=1.43 h. After a short hold at the solubility, the drug concentration in gastric fluid may rapidly drop to zero. Already by 3 hours after administering the dosage form the concentration may be less than 37 percent of the maximum.

Any other models for estimating the drug concentration in gastric fluid after administering particulate dosage forms obvious to a person of ordinary skill in the art are all within the spirit and scope of this disclosure.

### (d) Gastric Residence Time of Fibrous Dosage Form

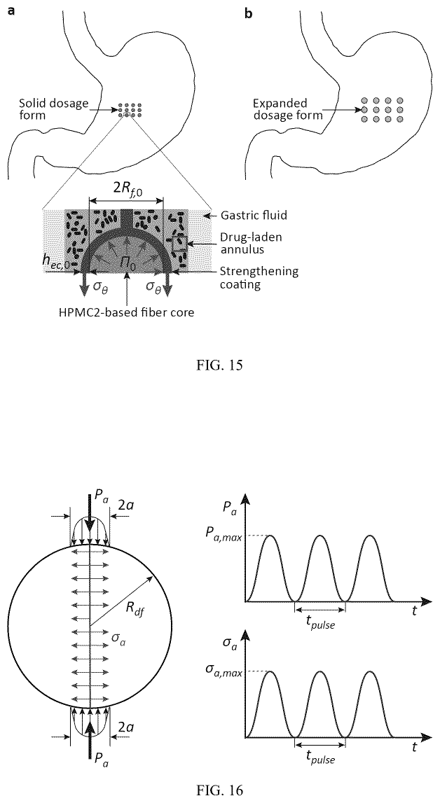

By contrast, the gastroretentive fibrous dosage form may expand in the stomach as shown schematically in the non-limiting FIG. 15 and detailed in subsection (b) of “Theoretical models of dosage form properties: In vitro expansion, mechanical properties, and drug release”. In the stomach, moreover, the expanded dosage form may be exposed to repeated compression pulses by the stomach walls. If the pulses are applied on diametrically opposed line contacts along the thickness of the dosage form, FIG. 16, the maximum applied tensile stress in the dosage form may roughly be estimated as:

$${σ}_{a , max} = \frac{{P}_{a , max}}{π {R}_{df}} \qquad ( 14 )$$

where P_{a,max} is the maximum load intensity (load per unit thickness) applied by the stomach walls, and R_{df} the radius of the expanded dosage form.

For non-limiting parameter values roughly resembling an expanded dosage form in an empty stomach of a dog, P_{a,max}˜1 N/mm and R_{df}˜10.5 mm, by Eq. (14) σ_{a,max}˜0.03 MPa. This is smaller than the estimated tensile stress of the dosage form, σ_{f,df}=0.175 MPa, calculated in subsection (c) of “Theoretical models of dosage form properties: In vitro expansion, mechanical properties, and drug release”. Thus, the expanded dosage form may remain in the stomach initially. Eventually, however, it may fracture or disintegrate due to dynamic fatigue or similar effects.

From companion work (see. e.g., REF. [3]), a highly approximate estimate of the time to fracture an expanded dosage form in dynamic fatigue may be written as:

$${t}_{f} = {{t}_{pulse} ( \frac{{σ}_{a , max}}{{σ}_{f , df}} )}^{1 / b} = {{t}_{pulse} ( \frac{{σ}_{a , max}}{{C}_{8} {φ}_{ec}^{n} {σ}_{ec}} )}^{1 / b} \qquad ( 15 )$$

where tₚᵤₗₛₑ is the period of the compression pulses by the stomach walls, σ_{a,max} the maximum tensile stress due to the compression pulses, σ_{f,df} tensile stress of the dosage form, b, C₈, and n are constants, φ_{ec} is the volume fraction of enteric coating in the solid dosage form, and σ_{ec} the fracture strength of the acidic water-soaked enteric coating.

Substituting the highly approximate, non-limiting values roughly resembling a fibrous dosage form in an empty stomach of a dog, tₚᵤₗₛₑ˜10 s, σ_{a,max}˜0.03 MPa, σ_{f,df}=0.175 MPa, and b˜−0.214 in Eq. (15), the time to fracture, t_{f} may be about 10 hours.

Fractured fragments that are smaller than the diameter of the pylorus may pass into the intestine with the gastric fluid flow. Fragments that are larger may continue to be subjected to the compressive loads by the stomach walls, and fracture eventually into smaller fragments that may flow out of the stomach.

The gastric residence time of the fibrous dosage form may be estimated as:

$${t}_{r , f} = {t}_{f} + {t}_{r , fr} \qquad ( 16 )$$

where t_{r,fr} is the characteristic time to decrease the amount of fragments in the stomach substantially. Substituting the non-limiting values, t_{f}˜10 h and t_{r,fr}˜1.43 h (the same as the estimated “gastric residence time” of drug particles in an empty stomach of a dog), by Eq. (16) t_{r,f}˜11.43 h. This is about an order of magnitude longer than the “gastric residence time” of drug particles estimated in subsection (b) above.

Any other models for estimating the gastric residence time of the disclosed dosage forms obvious to a person of ordinary skill in the art are all within the spirit and scope of this disclosure.

(e) Drug Concentration in Gastric Fluid after Administering Fibrous Dosage Forms

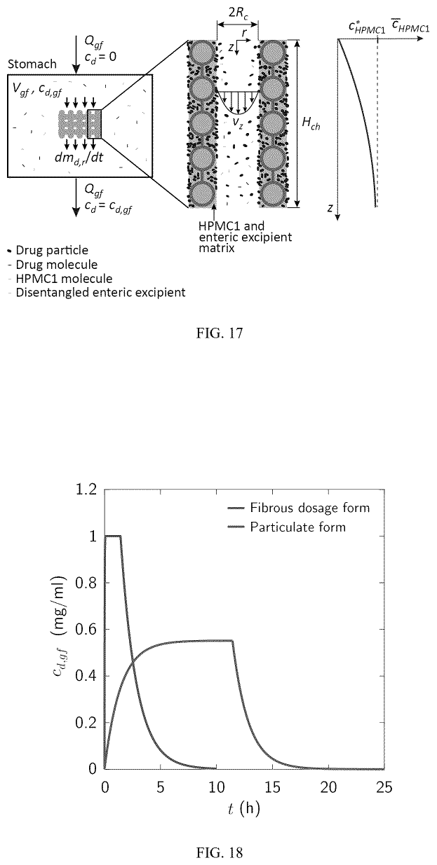

In the stomach, moreover, the fibrous dosage form may release drug as shown in the non-limiting FIG. 17. Assuming that no drug may be absorbed through the gastric wall, by mass balance the rate of change of the molecular concentration of drug in the gastric fluid may be written as:

$${V}_{gf} \frac{{dc}_{d , gf}}{dt} = - {Q}_{gf} {c}_{d , gf} + \frac{{dm}_{d , r}}{dt} \qquad ( 17 )$$

where V_{gf} is the volume of gastric fluid, Q_{gf} the flow rate of gastric fluid into and out of the stomach, and dm_{d,r}/dt the drug release rate by the dosage form.

From subsection (d) of section “Theoretical models of dosage form properties: In vitro expansion, mechanical properties, and drug release”, the drug release rate may be written as:

$$\frac{{dm}_{d , r}}{dt} = ( \frac{{w}_{d , a}}{{w}_{HPMC 1 , a}} ) {n}_{c} π {R}_{c}^{2} 〈 {v}_{z} 〉 {c}_{HPMC 1}^{*} = const \qquad ( 18 )$$

where w_{d,a} and w_{HPMC1,a}, respectively, are the weight fractions of drug and HPMC1 in the drug-containing annulus, n_{c} is the number of channels, R_{c} the radius of the channel in the expanded annulus, [symbol] v_{z} [symbol] the average velocity of the dissolution fluid through the channel, c_{HPMC1}* the HPMC1 concentration at the channel/annulus interface, and const a dimensional constant. For the non-limiting parameters, w_{d}=0.6, w_{HPMC1}=0.36, n_{c}=91.4, R_{c}=453 μm, [symbol] v_{z} [symbol] =6.68 μm/s, and c_{HPMC1}*=3.27 mg/ml, the drug release rate, dm_{d,r}/dt=const=7.72 mg/h.

It may be assumed here that this release rate is maintained as long as the fibrous dosage form resides in the stomach. By substituting dm_{d,r}/dt=const in Eq. (17), and solving the equation with the initial condition, c_{d,gf}=0 at t=0, up to the gastric residence time of the fibrous dosage form the drug concentration in gastric fluid may be obtained as (see. e.g., REF. [3]):

$$\begin{matrix}{c}_{d , gf} = \frac{const}{{Q}_{gf}} ( 1 - exp ( - \frac{{Q}_{gf} t}{{V}_{gf}} ) ) & 0 \leq t \leq {t}_{r , f}\end{matrix} \qquad ( 19 )$$

For t>t_{r,f}, however, dm_{d,r}/dt˜0. Substituting this release rate in Eq. (17), and solving the equation may give:

$$\begin{matrix}{c}_{d , gf} = {c}_{d , gf} {\vert}_{t = {t}_{r , f}} exp ( - \frac{{Q}_{gf} ( t - {t}_{r , f} )}{{V}_{gf}} ) & t > {t}_{r , f}\end{matrix} \qquad ( 20 )$$

where

$${c}_{d , gf} {\vert}_{t = {t}_{r , f}}$$

may be obtained from Eq. (19).

FIG. 18 plots Eqs. (19) and (20) for the non-limiting parameters const=7.72 mg/h, Q_{gf}=14 ml/h, V_{gf}=20 ml, and t_{r,f}=11.43 h. The drug concentration in the gastric fluid may rise exponentially with the time constant, τ_{gf}=V_{gf}/Q_{gf}˜1.43 h, and then it may asymptotically plateau out to the steady-state value, const/Q_{gf}˜0.55 mg/ml. After the gastric residence time (at 11.43 hours), the drug concentration in gastric fluid may fall exponentially with the same time constant, τ_{gf}=1.43 h.

Thus, in comparison with the particulate form, the drug concentration in the gastric fluid may be lower but it may be maintained for a longer time.

Any other models for estimating the drug concentration in gastric fluid after administering the disclosed dosage forms obvious to a person of ordinary skill in the art are all within the spirit and scope of this disclosure.

### (f) Drug Absorption by Duodenal Blood Capillaries

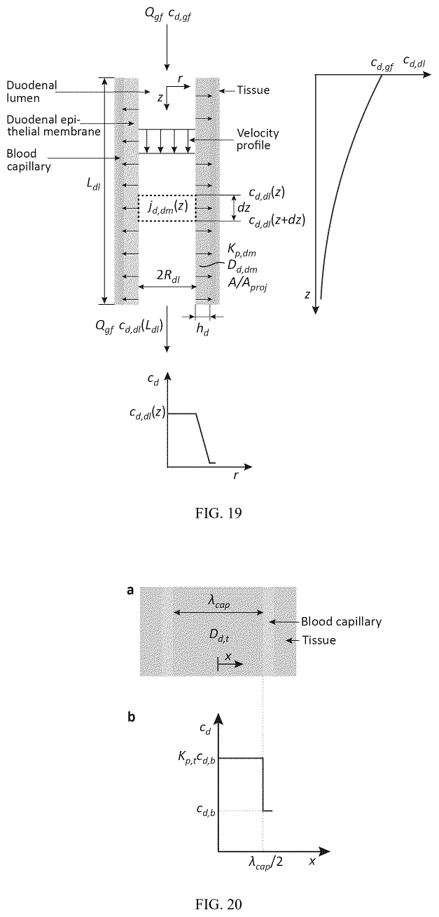

Drug molecules released in the stomach may pass into the duodenum (first part of the small intestine) and diffuse through the duodenal epithelial membrane into the blood capillaries, as shown in the non-limiting FIG. 19.

From companion work (see. e.g., REF. [3]), if the diffusivity of drug through the duodenal membrane is large, the number of drug molecules that exit the duodenum and enter the lower parts of the intestines may be very small. Further assuming that the drug molecules do not precipitate in the duodenum, the drug absorption rate by the blood may roughly be given by the mass flow rate at which drug molecules exit the stomach. Thus,

$$\frac{{dm}_{d , a}}{dt} = {Q}_{gf} {c}_{d , gf} \qquad ( 21 )$$

By Eq. (41) the absorption rate, dm_{d,a}/dt, may be directly proportional to the drug concentration in gastric fluid, c_{d,gf}. Thus, the absorption rate may be constant and/or controllable if the drug concentration in gastric fluid is constant and/or controllable.

Any other models for estimating drug absorption rate obvious to a person of ordinary skill in the art are all within the spirit and scope of this disclosure.

(g) Drug Diffusion from the Blood Capillaries into the Tissue and Vice Versa

The absorbed drug molecules may be transported forward with the blood along the duodenal or intestinal capillaries. Concurrently, they may diffuse from the capillaries into the surrounding tissue (cellular and extracellular space without blood), as shown schematically in the non-limiting FIG. 20a.

Shown schematically in the non-limiting FIG. 20b, is a longitudinal section along two parallel neighboring capillaries spaced apart by a tissue slab of thickness, λ_{c}. Assuming that drug transport in the tissue is fast, the drug concentration in the tissue may be quasi-steady and roughly at equilibrium with the drug concentration in blood.

The drug concentration in the tissue may then be estimated as:

$${c}_{d , t} = {K}_{p , t} {c}_{d , b} \qquad ( 22 )$$

where K_{p,t} is the tissue-blood partition coefficient and c_{d,b} the drug concentration in blood.

Thus, the drug concentration in the tissue may rise and fall with the drug concentration in blood. A non-limiting value of K_{p,t} for nilotinib in a dog, K_{p,t}=2.08.

Any other models for determining drug distribution into tissues obvious to a person of ordinary skill in the art are all within the spirit and scope of this disclosure.

### (h) Drug Elimination in the Liver

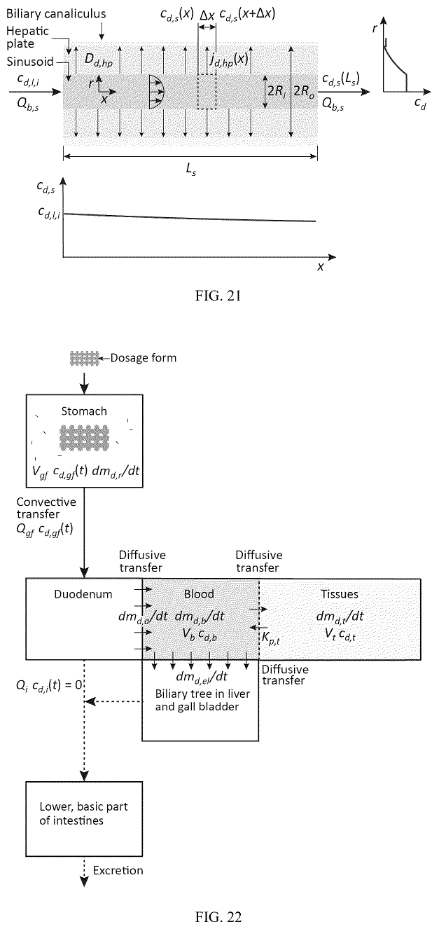

The absorbed drug molecules in the blood capillaries of the duodenum may further be convected into the portal vein and the sinusoidal blood capillaries of the liver. In the sinusoidal capillaries, the drug may be continuously eliminated from the blood by diffusion through the hepatic plates into the biliary canaliculi, as shown in the non-limiting FIG. 21.

From companion work (see, e.g., REF. [3]), a rough estimate of the elimination rate of drug molecules by the liver may be written as:

$$\frac{{dm}_{d , el}}{dt} = 2 π \frac{{K}_{p , hp} {D}_{d , hp} {L}_{s}}{ln ( {R}_{o} / {R}_{i} ) {Q}_{b , s}} {Q}_{b , l} {c}_{d , b} \qquad ( 23 )$$

where K_{p,hp}=(c_{d,hp}/c_{d,b})_{r=Ri} is the partition coefficient of the drug between the hepatic plates and the blood in the sinusoidal capillary, D_{d,ph} the drug diffusivity through the hepatic plates, Lₛ the length of the sinusoids, Rₒ the outer radius of the hepatic plates, Rᵢ the radius of the sinusoidal capillaries, Q_{b,s} the flow rate of blood through a sinusoidal capillary, Q_{b,l} the flow rate of blood through the liver, and c_{d,b} the drug concentration in blood.

Eq. (23) suggests that the drug elimination rate may be roughly proportional to the drug concentration in blood.

Any other models for estimating the drug elimination rate from the blood obvious to a person of ordinary skill in the art are all within the spirit and scope of this disclosure.

### (i) Drug Concentration in Blood Versus Time

Finally, the models of drug absorption, drug distribution, and drug elimination may be combined for expressing the drug concentration in the blood versus time. As shown schematically in FIG. 22 and detailed in a companion work (see, e.g., REF. [3]), assuming that the drug concentrations in the blood and in the tissue are uniform throughout the body, by mass balance an approximate equation for the drug concentration in blood may be obtained as:

$$( {V}_{p} + {K}_{p , t} {V}_{t} ) \frac{{dc}_{d , b}}{dt} = \frac{{dm}_{d , a}}{dt} - \frac{{dm}_{d , el}}{dt} \qquad ( 24 )$$

where V_{b} is the volume of blood and Vₜ the volume of tissue in the body.

Substituting Eq. (21) for dm_{d,a}/dt and Eq. (23) for dm_{d,el}/dt in Eq. (24) may give:

$$( {V}_{b} + {K}_{p , t} {V}_{t} ) \frac{{dc}_{d , b}}{dt} = {Q}_{gf} {c}_{d , gf} - 2 π \frac{{K}_{p , hp} {D}_{d , hp} {L}_{s}}{ln ( {R}_{o} / {R}_{i} ) {Q}_{b , s}} {Q}_{b , l} {c}_{d , b} \qquad ( 25 )$$

Rearranging and rewriting:

$$\frac{{dc}_{d , b}}{dt} + \frac{{c}_{d , b}}{{τ}_{el}} = \frac{{Q}_{gf} {c}_{d , gf}}{{V}_{b} + {K}_{p , t} {V}_{t}} \qquad ( 26 a )$$

where τₑₗ may be considered an elimination time constant, given by:

$${τ}_{el} = \frac{( {V}_{b} + {K}_{p , t} {V}_{t} ) ln ( {R}_{o} / {R}_{i} ) {Q}_{b , s}}{2 π {K}_{p , hp} {D}_{d , hp} {L}_{s} {Q}_{b , l}} \qquad ( 26 b )$$

For the non-limiting parameter values V_{b}=1.1 l, K_{p,t}=2.08, Vₜ=12.8 l, Rₒ=15 μm, Rᵢ=5 μm, Q_{b,s}=3.93×10⁻⁸ ml/s, K_{p,hp}=2.08, D_{d,hp}=5.24×10⁻¹² m²/s, Lₛ=275 μm, and Q_{b,l}: =9 ml/s, by Eq. (26b) τₑₗ=1.96 h.

In the following subsections, the drug concentration in gastric fluid derived in the earlier sections is substituted in Eq. (26a), and the drug concentration in blood is estimated for both the particulate and the fibrous dosage forms. The solution may be divided into two time intervals: Before and after the gastric residence time.

(i.1) Drug Concentration in Blood after Administering Particulate Dosage Forms

$$0 \leq t \leq {t}_{r , p} \qquad ( i \text{.1} . a )$$

Up to the gastric residence time of drug particles, t_{r,p}˜V_{gf}/Q_{gf}˜1.43 h, the drug concentration in gastric fluid, by Eq. (12) may be about equal to the solubility. Substituting c_{d,gf}=c_{s,gf} in Eq. (63a) and solving the equation with the initial condition, c_{d,b}=0 at t=0, may give (see, e.g., REF. [3]):

$$\begin{matrix}{c}_{d , b} = \frac{{Q}_{gf} {c}_{s , gf} {τ}_{el}}{{V}_{b} + {K}_{p , t} {V}_{t}} ( 1 - exp ( - \frac{t}{{τ}_{el}} ) ) & 0 \leq t \leq {t}_{r , p}\end{matrix} \qquad ( 27 a )$$

Because the “gastric residence time” of the drug particles, t_{r,p}=1.43 h, is shorter than the time constant, τₑₗ=1.96 h, up to t_{r,p} the exponential term in Eq. (27a) may be expanded as 1−t/τₑₗ. Substituting this term for the exponential term gives:

$${c}_{d , b} = \frac{{Q}_{gf} {c}_{s , gf}}{{V}_{b} + {K}_{p , t} {V}_{t}} t \qquad ( 27 b )$$

FIG. 13 plots Eq. (27a) for the non-limiting parameter values, Q_{gf}=14 ml/h, c_{s,gf}=1 mg/ml, V_{b}=1.1 l, K_{p,t}=2.08, Vₜ=12.8 l, τₑₗ=1.96 h, and t_{r,p}=1.43 h. Indeed, up to t_{r,p} the drug concentration in blood may increase roughly linearly with time.

$$t > {t}_{r , p} \qquad ( i \text{.1} . b )$$

After the gastric residence time of drug particles, by substituting Eq. (13) in Eq. (26a), and solving the equation with c_{d,b} at t=t_{r,p} obtained from Eq. (27a), the drug concentration in blood may be obtained as (see, e.g., REF. [3]):

$$\begin{matrix}{c}_{d , b} = \frac{{Q}_{gf} {c}_{s , gf} {τ}_{el}}{{V}_{b} + {K}_{p , t} {V}_{t}} \times ( \frac{{τ}_{el}}{{τ}_{el} - {τ}_{gf}} exp ( - \frac{t - {t}_{r , p}}{{τ}_{el}} ) - \frac{{τ}_{gf}}{{τ}_{el} - {τ}_{gf}} exp ( - \frac{t - {t}_{r , p}}{{τ}_{gf}} ) - exp ( - \frac{t}{{τ}_{el}} ) ) & t > {t}_{r , p}\end{matrix} \qquad ( 28 )$$

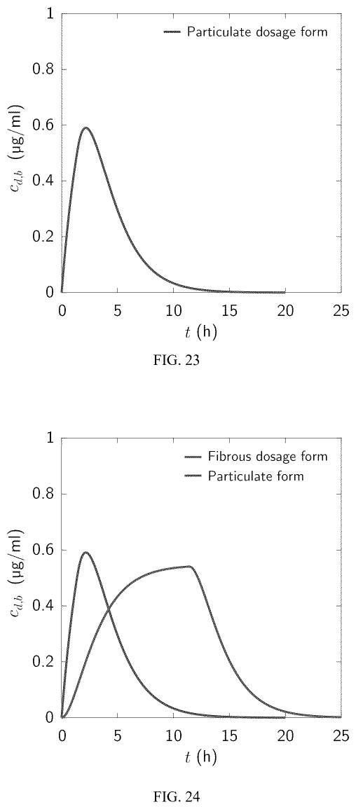

FIG. 23 plots Eq. (28) for times, t>t_{r,p}, and the non-limiting parameter values Q_{gf}=14 ml/h, c_{s,gf}=1 mg/ml, V_{b}=1.1 l, K_{p,t}=2.08, Vₜ=12.8 l, τₑₗ=1.96 h, τ_{gf}=1.43 h, and t_{r,p}=1.43 h. Because τₑₗ>τ_{gf}, the first term in the parentheses may dominate, and the drug concentration may fall exponentially with the time constant, τₑₗ.

Therefore, because t_{r,p} is short, after administering the particulate dosage form the drug concentration in blood may rise and fall. As listed in Table 2, the maximum drug concentration, cₘₐₓ˜0.59 μg/ml at tₘₐₓ˜2.2 h. The width of the peak at half-height, w_{1/2}˜4.3 hours. This is much shorter than the reasonable dosing intervals, 12 or 24 hours.

Any other models for estimating the drug concentration in blood after administering particulate dosage forms obvious to a person of ordinary skill in the art are all within the spirit and scope of this disclosure.

(i.2) Drug Concentration in Blood after Administering Fibrous Dosage Forms

$$0 \leq t \leq {t}_{r , f} \qquad ( i \text{.2} . a )$$

Up to the gastric residence time of the fibrous dosage form, t_{r,f}˜11.43 h, by substituting Eq. (19) in Eq. (26a) and solving the equation with the initial condition, c_{d,b}=0 at t=0, the drug concentration in blood may be obtained as (see, e.g., REF. [3]):

$$\begin{matrix}{c}_{d , b} = \frac{const {τ}_{el}}{{V}_{b} + {K}_{p , t} {V}_{t}} ( 1 + ( \frac{{τ}_{gf}}{{τ}_{el} - {τ}_{gf}} ) exp ( - \frac{t}{{τ}_{gf}} ) - ( \frac{{τ}_{el}}{{τ}_{el} - {τ}_{gf}} ) exp ( - \frac{t}{{τ}_{el}} ) ) & 0 \leq t \leq {t}_{r , f}\end{matrix} \qquad ( 29 )$$

FIG. 24 plots Eq. (29) for the non-limiting parameter values const=7.72 mg/h, τₑₗ=1.96 h, V_{b}=1.1 l, K_{p,t}=2.08, Vₜ=12.8 l, τ_{gf}=1.43 h, and t_{r,f}=11.43 h. Because τₑₗ>τ_{gf}, the drug concentration may rise with the time constant, τₑₗ˜1.96 hours. Moreover, because τₑₗ<<t_{r,f}, the drug concentration in blood may plateau to the steady state value, cₛₛ=constτₑₗ/(V_{b}+K_{p,t}Vₜ)˜0.55 μg/ml.

$$t > {t}_{r , f} \qquad ( i \text{.2} . b )$$

After the gastric residence time of the fibrous dosage form, substituting Eq. (20) in Eq. (26a), and solving the equation with c_{d,b} at t=t_{r,f} obtained from Eq. (29), the drug concentration in blood may be obtained as (see, e.g., REF. [3]):

$$\begin{matrix}{c}_{d , b} = {c}_{d , b} {\vert}_{t = {t}_{r , f}} exp ( - \frac{t - {t}_{r , f}}{{τ}_{el}} ) + \frac{const {τ}_{el}}{{V}_{b} + {K}_{p , t} {V}_{t}} ( \frac{{τ}_{gf}}{{τ}_{el} - {τ}_{gf}} ) \times ( exp ( - \frac{t - {t}_{r , f}}{{τ}_{el}} ) - exp ( - \frac{t - {t}_{r , f}}{{τ}_{gf}} ) ) & t > {t}_{r , f}\end{matrix} \qquad ( 30 )$$

FIG. 24 plots Eq. (30) for times, t>t_{r,f} and the non-limiting parameter values const=7.72 mg/h, τₑₗ=1.96 h, V_{b}=1.1 l, K_{p,t}=2.08, Vₜ=12.8 l, τ_{gf}=1.43 h, and t_{r,f}=11.43 h. Thus, after administering the fibrous dosage forms the drug concentration in blood may rise, plateau out, and fall. The peak-width at half-height estimated from the models is 11.5 hours, 2.7 times that of the particulate forms, and about the reasonable dosing interval, Table 1.

TABLE 2
Calculated in vivo properties of particulate and fibrous dosage forms.

| | t_{r,p} | t_{r,f} | cₘₐₓ | tₘₐₓ | w_{1/2} |
|---|---|---|---|---|---|
| | (h) | (h) | (μg/m) | (h) | (h) |
| Particulate forms | 1.43 | — | 0.59 | 2.2 | 4.3 |
| Fibrous forms | — | 11.43 | 0.54 | 11.43 | 11.5 |

t_{r,p}: gastric residence time of drug particles;
t_{r,f.} gastric residence time of fibrous dosage forms;
cₘₐₓ: maximum drug concentration in blood;
tₘₐₓ: time when drug concentration in blood is maximal;
w_{1/2}: width of peak at half height.
t_{r,p} is calculated by Eq. (11).
t_{r,f} is calculated by Eqs. (15) and (16).
cₘₐₓ, tₘₐₓ, and w_{1/2} are obtained from FIGS. 23 and 24.

Thus, upon repeated dosing the fibrous dosage form may be able to maintain a fairly steady drug concentration in blood—enhancing efficacy and mitigating side effects of the drug therapy.

Any more models and concepts of demonstrating superiority of the disclosed dosage forms to state-of-the-art or other dosage forms obvious to a person of ordinary skill in the art are all within the spirit and scope of this invention.

### Additional Embodiments of the Dosage Form

In view of the theoretical models and non-limiting examples above, which are suggestive and approximate rather than exact, and other considerations, the dosage forms disclosed herein may further comprise the following embodiments.

### (a) Outer Geometry of Dosage Form, Fluid-Absorptive, Expandable Solid Core, Expandable Three-Dimensional Structural Framework, or Expandable, Drug Releasable Solid

Generally, the dosage forms disclosed herein may be used for delivery of drugs by oral ingestion. Thus, to assure that the dosage form is swallowable by a human or animal subject, the exterior dimensions of the dosage form (or of a fluid-absorptive, expandable solid core, or of an expandable, three-dimensional structural framework of one or more fluid-absorptive structural elements, or of an encapsulated expandable, three-dimensional structural framework of one or more fluid-absorptive structural elements, or of an expandable, drug releasable solid, etc.) disclosed herein may not be too large. In some embodiments, therefore, at least one exterior dimension of the dosage form or of a fluid-absorptive, expandable solid core or of an expandable, three-dimensional structural framework of one or more fluid-absorptive structural elements or of an expandable, drug releasable solid herein may be no greater than 16 mm. This includes, but is not limited to at least one exterior dimension of the dosage form or of a fluid-absorptive, expandable solid core of an expandable, three-dimensional structural framework of one or more fluid-absorptive structural elements or of an expandable, drug releasable solid no greater than 15 mm, or no greater than 14 mm, or no greater than 13 mm, or no greater than 12 mm or no greater than 11 mm, or no greater than 10 mm.

After ingestion, however, the dosage form (or fluid-absorptive, expandable solid core or expandable, drug releasable solid) should remain in the stomach for prolonged time. Thus, to prevent premature passage into the small intestine, the dosage form (or fluid-absorptive, expandable solid core or expandable, drug releasable solid) disclosed herein may expand in the stomach to a size of the order of or greater than the diameter of the pylorus (e.g., the diameter of the pyloric sphincter, etc.). Because the maximum expansion and the expansion rate may be limited, dosage forms (or fluid-absorptive, expandable solid cores or expandable, three-dimensional structural frameworks of one or more fluid-absorptive structural elements or expandable, drug releasable solids) with greater exterior dimensions may be preferred. In some embodiments, therefore, at least one exterior dimension of the dosage form or of a fluid-absorptive, expandable solid core or of an expandable, three-dimensional structural framework of one or more fluid-absorptive structural elements or of an expandable, drug releasable solid herein may be greater than 3 mm. This includes, but is not limited to at least one exterior dimension of the dosage form or of a fluid-absorptive, expandable solid core or of an expandable, three-dimensional structural framework of one or more fluid-absorptive structural elements or of an expandable, drug releasable solid greater than 4 mm, or greater than 5 mm, or greater than 6 mm, or greater than 7 mm, or greater than 8 mm, or greater than 9 mm.

In some embodiments, therefore, at least one exterior dimension of the dosage form or of a fluid-absorptive, expandable solid core or of an expandable, three-dimensional structural framework of one or more fluid-absorptive structural elements or of an expandable, drug releasable solid herein may be in the range of 3 mm-16 mm. This includes, but is not limited to at least one exterior dimension of the dosage form or of a fluid-absorptive, expandable solid core or of an expandable, drug releasable solid in the ranges 4 mm-16 mm, 5 mm-16 mm, 6 mm-16 mm, 5 mm-16 mm, or 6 mm-15 mm.

The dosage form (or fluid-absorptive, expandable solid core or expandable, three-dimensional structural framework of one or more fluid-absorptive structural elements or expandable, drug releasable solid) disclosed herein can have any common or uncommon outer shape of oral solid dosage forms (e.g., tablets, capsules, etc.). For non-limiting examples of common tablet shapes, see, e.g., K. Alexander, Dosage forms and their routes of Administration, in M. Hacker, W. Messer, and K. Bachmann, Pharmacology: Principles and Practice, Academic Press, 2009. Any other outer geometries, outer shapes, outer surfaces, or dimensions of dosage forms or fluid-absorptive, expandable solid cores or expandable, three-dimensional structural frameworks of one or more fluid-absorptive structural elements or expandable, drug releasable solids obvious to a person of ordinary skill in the art are all within the spirit and scope of this invention.

### (b) Structure of Fluid-Absorptive, Expandable Solid Core and Three Dimensional Structural Framework of Elements or Fibers

For assuring that the dosage form (or an expandable, drug releasable solid) herein comprises the desired properties, such as the desired expansion rate, adequate mechanical properties after expansion, the desired drug release rate, and so on, in some embodiments a fluid-absorptive solid core as disclosed herein may comprise a three dimensional structural framework of one or more fluid-absorptive elements. The three dimensional structural framework of elements may comprise segments separated and spaced from adjoining segments by element-free spacings. Such element-free spacings may define one or more element-free spaces within the exterior (or outer) volume of the three dimensional structural framework of elements.

If such element-free spaces are reasonably well connected within or through an exterior volume of a three dimensional structural framework of elements, they may enable percolation of a fluid into one or more of said element-free spaces upon immersing said framework into said fluid.

Percolation of fluid into one or more element-free spaces can be desirable for various reasons. By way of example but not by way of limitation, a non-limiting method or way of manufacturing dosage forms as disclosed herein includes dip-coating a structural framework of fluid-absorptive elements with an encapsulating mechanically strengthening coating. If a dip-coating solution (e.g., a solution comprising at least a coating substance and a solvent) percolates the element-free spaces surrounding said framework, the coating may encapsulate said framework. The more connected the element-free spaces, the more uniformly may the dip-coating solution percolate the element-free spaces, and the more uniformly may the mechanically strengthening coating be applied around said framework. A more uniform coating may result in improved properties of the dosage form or expandable, drug releasable solid, such as greater mechanical strength after expansion, etc.

In some embodiments, therefore, one or more element-free spaces within an exterior volume of a three-dimensional structural framework of elements may be substantially connected through an exterior dimension of said structural framework. Similarly, in some embodiments, one or more fiber-free spaces within an exterior volume of a three-dimensional structural framework of criss-crossed stacked layers of one or more fibers may be substantially connected through an exterior dimension of said structural framework.

In some embodiments of the invention herein, one or more element-free spaces within an exterior volume of a three dimensional structural framework of elements may be considered “substantially connected” if a fluid can substantially percolate said one or more element-free spaces upon immersing said framework in said fluid.

In some embodiments of the invention herein, moreover, one or more element-free spaces within an exterior volume of a three dimensional structural framework of elements may be considered “substantially connected” if they extend over a length at least about a third of an exterior dimension of said structural framework. This includes, but is not limited to one or more element-free spaces within an exterior volume of a three-dimensional structural framework of elements extending over a length at least two thirds of an exterior dimension of said structural framework, or over a length at least equal to an exterior dimension of said structural framework, or over a length and width at least equal to an exterior dimension of said structural framework, or over the entire length, width, and thickness of the exterior volume of said three-dimensional structural framework.

In the invention herein, one or more element-free spaces that are “connected” may also be referred to as one or more element-free spaces that are “interconnected”, “contiguous”, “in direct contact”, “merged”, “without any wall in between”, and so on. Similarly, an “exterior dimension of a structural framework” may be considered a “length of an exterior volume of said structural framework”, and so on.

FIG. 25 schematically illustrates a non-limiting solid core 2505 comprising an exterior surface 2502 and an internal, three dimensional structural framework 2504 of a plurality of criss-crossed stacked layers of fibrous structural elements 2510. Said framework 2504 is contiguous with and terminates at said exterior surface 2502. The fibrous structural elements 2510 further have segments spaced apart from adjoining segments, thereby defining element-free or fiber-free spaces 2520. A plurality of adjacent element-free or fiber-free spaces 2525 combine to define one or more interconnected element-free or fiber-free spaces 2530.

As shown in the non-limiting schematic of section A-A, at least one interconnected element-free or fiber-free space 2530 extends over the entire length and thickness of the outer or exterior volume of the three dimensional structural framework 2504. In other words, the length, L_{ef}, over which the interconnected element-free or fiber-free space 2530 extends is the same or about the same as the length of the outer or exterior volume of the three dimensional structural framework 2504; the thickness, H_{ef}, over which the interconnected element-free or fiber-free space 2530 extends is the same or about the same as the thickness, H, of the outer or exterior volume of the three dimensional structural framework 2504. It may be noted that the term “section” is understood herein as “plane” or “surface”. Thus a “section” is not a “projection” or “projected view”.

Moreover, in the non-limiting example of FIG. 25 the microstructure is rotationally symmetric. If the plane or section A-A is rotated by 90 degrees about the central axis the microstructure (e.g., the microstructural details) is/are the same. Thus, the interconnected element-free or fiber-free space 2530 also extends over the entire width of the outer or exterior volume of the three dimensional structural framework 2504 or solid core 2505. In other words, the width over which the interconnected element-free or fiber-free space 2530 extends is the same or about the same as the width of the outer or exterior volume of the three dimensional structural framework 2504 or solid core 2405.

Furthermore, in the non-limiting microstructure of FIG. 25, as shown in section A-A the interconnected element-free or fiber-free space 2530 (or element-free or fiber-free space 1320 or element-free or fiber-free spaces 2525) is/are contiguous. No walls (e.g., walls comprising the three dimensional structural framework 2504 of elements) must be ruptured to obtain an interconnected cluster of element-free or fiber-free spaces (e.g., an open channel of element-free or fiber-free space) from the outer or exterior surface 2502 of the three dimensional structural framework 2504 to a point (or to any point or position) in the element-free or fiber-free space 2520, 2525, 2530. Also, no walls (e.g., walls comprising the three dimensional structural framework 2504 of elements) must be ruptured to obtain an interconnected cluster of element-free or fiber-free space (e.g., an open channel of element-free or fiber-free space) from any point or position within the element-free or fiber-free space 1320, 2525, 2530 to any other point or position in the element-free or fiber-free space 2520, 2525, 2530. The entire element-free or fiber-free space 2520, 2525, 2530 is accessible from the outer or exterior surface 2502 of the three dimensional structural framework of fibers 2504 or solid core 2505. In addition, the entire element-free or fiber-free space 2520, 2525, 2530 is accessible from any point, location, or position within the element-free or fiber-free space 2520, 2525, 2530.

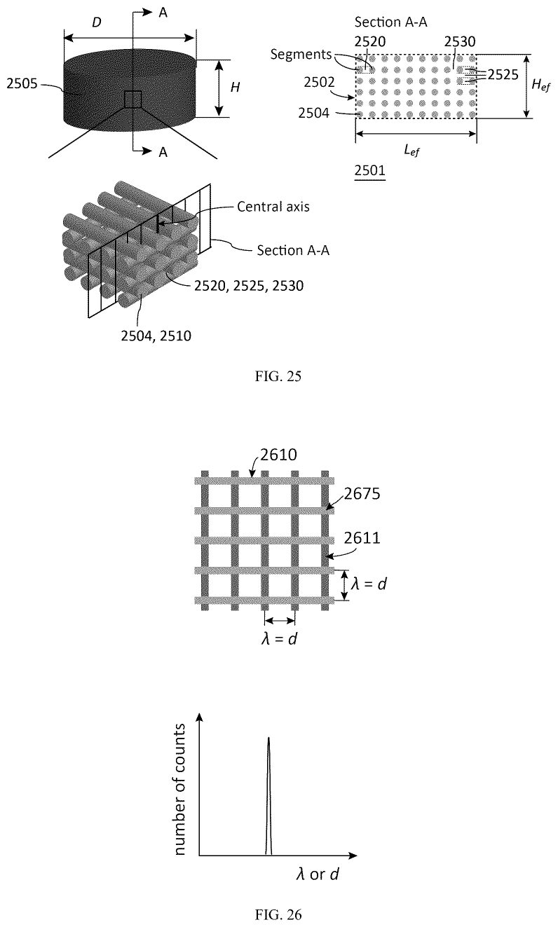

Several microstructural features can be defined to further characterize such structures. By way of example but not by way of limitation, as shown in FIG. 26, the structural framework 2610 may be considered a network comprising nodes or vertices at the inter-fiber contacts 2675 and edges 2611 defined by the fiber segments of length, λ, between adjacent nodes or vertices 2675.

FIG. 26 also shows a histogram of the length, λ, of fiber segments between adjacent point contacts 2675. The λ values in this non-limiting example are distributed in a very narrow window or zone around the average, λ_{avg}. Thus, the standard deviation of the λ values is very small; λ is precisely controlled; the structure is repeatable, regular, deterministic, and ordered. Generally, structural elements may be understood as “orderly arranged” or “repeatably arranged” if such structural features as spacing between elements, orientation of elements, etc. is/are controlled. A structural feature may be referred to as “controlled” if a standard deviation of said feature across a three dimensional structural framework (or across multiple three dimensional structural frameworks of multiple dosage forms, etc.) is smaller (or much smaller) than that in a random structure with randomly arranged elements. Generally, moreover, the properties of ordered structures may be controllable or better controllable than those or random structures.

In some embodiments, therefore, one or more structural elements or segments thereof may be repeatably or substantially repeatably arranged. More specifically, in some embodiments, one or more structural elements or segments thereof may be repeatably or substantially repeatably arranged within a three dimensional structural framework formed by said one or more elements or segments. Similarly, in some embodiments one or more structural elements or segments thereof may be orderly or substantially orderly arranged. More specifically, in some embodiments, one or more structural elements or segments thereof may be orderly or substantially orderly arranged within a three dimensional structural framework formed by said one or more elements or segments.

Also, in some embodiments one or more fibers or segments thereof may be orderly or substantially orderly arranged in a three dimensional structural framework of criss crossed stacked layers of one or more fibers. Similarly, in some embodiments one or more fibers or segments thereof forming a three dimensional structural framework of criss-crossed stacked fiber layers may be substantially orderly arranged.

It may be noted, furthermore, that in embodiments where a three dimensional structural framework of elements comprises criss-crossed stacked layers of fibers, the inter-fiber spacing in a layer may be uniform or substantially uniform. In some embodiments, furthermore, the inter-fiber spacing in the fiber layers (e.g., in all fiber layers, etc.) of a structural framework of criss-crossed stacked fiber layers may be substantially the same and/or substantially uniform within the exterior volume of said framework.

In some embodiments, moreover, one or more fibers or fiber segments in a layer of a three dimensional structural framework of criss-crossed stacked fiber layers may be substantially parallel.

In some embodiments moreover, at least four (e.g., at least five, at least six, at least seven, etc.) substantially flat layers of one or more fibers or fiber segments may be stacked in a cross-ply arrangement to form a three dimensional structural framework.

In some embodiments, moreover a three dimensional structural framework may comprise a single continuous structure.

Moreover as the theoretical models and the experimental examples suggests, a relevant parameter for determining the expansion rate and other properties of a dosage form (or of a fluid-absorptive, expandable solid core or of an expandable, drug releasable solid) herein may be the volume fraction of one or more fluid-absorptive elements in an outer volume of a three dimensional structural framework formed by said one or more elements. In some embodiments, a larger volume fraction of fluid-absorptive elements may increase the expansion rate of a dosage form (or of a fluid-absorptive, expandable solid core or of an expandable, drug releasable solid) and enhance some mechanical properties, such as an elastic modulus, of an expanded dosage form or an expanded drug releasable solid.

In some embodiments, therefore, a three dimensional structural framework of one or more elements comprises an outer surface and an outer volume, said outer volume defined by the volume enclosed (e.g., enveloped, etc.) by said outer surface. In some embodiments, the volume fraction of one or more fluid-absorptive elements within an outer volume of a three dimensional structural framework formed by said one or more elements may be greater than 0.01. This includes, but is not limited to a volume fraction of one or more fluid-absorptive elements within an outer volume of a three dimensional structural framework formed by said one or more elements greater than 0.03, or greater than 0.05, or greater than 0.07, or greater than 0.08, or greater than 0.1, or greater than 0.12, or greater than 0.14, or greater than 0.15, or greater than 0.17, or greater than 0.18, or greater than 0.2.

The volume fraction of one or more fluid-absorptive elements within an outer volume of a three dimensional structural framework formed by said one or more elements may, however, also not be too large to assure that the dosage form or drug releasable solid possesses the desired properties. In some embodiments, therefore, the volume fraction of one or more fluid-absorptive elements within an outer volume of a three dimensional structural framework formed by said one or more elements is in the range between 0.005 and 0.75. This includes, but is not limited to a volume fraction of one or more fluid-absorptive elements within an outer volume of a three dimensional structural framework formed by said one or more elements in the ranges 0.01-0.75, or 0.02-0.075, or 0.005-0.7, or 0.01-0.7, or 0.02-0.7.

Furthermore, it may be noted that many of the above features and characteristics that were presented here for fibrous structures may also apply to (e.g., the features or characteristics may be similar to the features or characteristics of) three-dimensional structural frameworks of stacked layers of sheets, or stacked layers of beads (or particles), as shown, for example, in the International Application No. PCT/US2019/052030 filed on Sep. 19, 2019, and titled “Dosage form comprising structured solid-solution framework of sparingly-soluble drug and method for manufacture thereof”. Such features or characteristics are obvious to a person of ordinary skill in the art who is given all information disclosed in this specification. Application of such features or characteristics to three-dimensional structural frameworks of stacked layers of beads (or particles), or stacked layers sheets (or two-dimensional elements), is included in the invention herein.

Any more microstructures of solid cores and three dimensional structural frameworks of elements or fibers would be obvious to a person of ordinary skill in the art. All of them are included in this invention.

### (c) Element-Free or Fiber-Free Spacing

To assure that fluid can percolate element-free space and/or to assure that element-free space can be adequately occupied by encapsulating coating, drug-containing solid, and/or free space into which a physiological fluid can percolate, the element-free spacing (e.g., the channel size or diameter, channel width, pore size, etc.) between elements, fibers, or segments may be on the micro- or macro-scale. Thus, in some embodiments, the element-free spacing, λ_{f,e}, between elements (e.g., fibers) or segments through (e.g., across, along, etc.) one or more element-free spaces or through (e.g., across, along, etc.) one or more connected element-free spaces (e.g., the channel size or channel diameter) is greater than 10 μm. This includes, but is not limited to λ_{f,e} greater than 15 μm, or greater than 20 μm, or greater than 25 μm, or greater than 30 μm, or greater than 35 μm, or greater than 40 μm, or greater than 45 μm, or greater than 50 μm, or greater than 60 μm, or greater than 70 μm, or greater than 80 μm, or greater than 90 μm, or greater than 100 μm, or greater than 125 μm, or greater than 150 μm, or greater than 175 μm, or greater than 200 μm.

For assuring that the dosage form has the required properties (e.g., the required mechanical properties), however, the element-free spacing may also not be too large. Thus, in some embodiments, the element-free spacing through (e.g., across, along, etc.) one or more element-free spaces or through (e.g, across, along, etc.) a connected element-free space may be in the ranges 10 μm-5 mm, 10 μm-3 mm, 15 μm-5 mm, 25 μm-5 mm, 25 μm-3 mm, 50 μm-4.5 mm, 50 μm-4 mm, 100 μm-5 mm, 100 μm-4 mm, 200 μm-5 mm, 150 μm-4 mm, 200 μm-4 mm, 50 μm-4 mm, 40 μm-4 mm, or 100 μm-5 mm.

Moreover, in some embodiments herein one or more element-free spacings between one or more elements or segments within the outer volume of a three dimensional structural framework may be precisely controlled. Similarly, in some embodiments herein one or more element-free spacings between one or more elements or segments through (e.g., across, along, etc.) one or more interconnected element-free spaces may be precisely controlled.

The element-free spacing may be determined experimentally from microstructural images (e.g., scanning electron micrographs, micro computed tomography scans, and so on) of a three dimensional structural framework of elements or of an expandable, drug releasable solid or dosage form. Non-limiting examples describing and illustrating how an element-free spacing may be determined from microstructural images are given in the U.S. application Ser. No. 15/482,776 titled “Fibrous dosage form”.

Any more details of element-free or fiber-free spacings would be obvious to a person of ordinary skill in the art. All of them are included in this invention.

### (d) Thickness of Elements or Fibers

To assure that the dosage form or expandable, drug releasable solid disclosed herein may achieve several properties, such as fast expansion after immersing in a physiological fluid under physiological conditions, adequate drug release rate, and so on, a large specific surface area (i.e., a large surface area-to-volume ratio) of the framework of elements can be desirable. In some embodiments, therefore, one or more elements (e.g., one or more fibers) forming a three-dimensional structural framework may have an average thickness, h₀, no greater than 2.5 mm. This includes, but is not limited to h₀ no greater than 2 mm, or no greater than 1.75 mm, or no greater than 1.5 mm, or no greater than 1.25 mm, or no greater than 1 mm, or no greater than 750 μm.

It may be noted, however, that if the elements are very thin and tightly packed, the spacing and free spacing between the elements may be too small. Thus, in some embodiments one or more elements (e.g., one or more fibers) forming a three dimensional structural framework of said one or more elements may have an average thickness, h₀, in the range of 5 μm-2.5 mm. This includes, but is not limited to average thickness, h₀, of one or more elements (e.g., one or more fibers) forming a three dimensional structural framework of said one or more elements in the ranges 10 μm-2 mm, 20 μm-2 mm, 25 μm-2 mm, 30 μm-2 mm, 20 μm-1.5 mm, 25 μm-1.5 mm, 25 μm-1.25 mm, 25 μm-1 mm, 30 μm-1.5 mm, 30 μm-1.25 mm, 30 μm-1 μm, 40 μm-1.5 mm, or 50 μm-1.25 mm.

In some embodiments, moreover, the average thickness of the one or more elements (e.g., fibers) comprising or composing (e.g., producing, making up, etc.) a three dimensional structural framework or network (e.g., the average thickness of the elements in a three dimensional structural network) may be precisely controlled.

The element or fiber thickness, h, may be considered the smallest dimension of an element (i.e., h≤w and h≤l, where h, w and l are the thickness, width and length of the element, respectively). The average element or fiber thickness, h₀, is the average of the element or fiber thickness along the length of the one or more elements or fibers. A non-limiting example illustrating how an average fiber thickness may be derived is presented in U.S. application Ser. No. 15/482,776 titled “Fibrous dosage form”.

In some embodiments, at least one outer surface of one or more elements (e.g., the outer surface or one or more fibers or the outer surface of a fiber segment) comprises a coating. Said coating may cover part of or the entire outer surface of one or more elements or a segment thereof. Said coating may further have a composition that is different from the composition of one or more elements or a segment thereof. The coating may be a solid, and may or may not comprise or contain a drug.

Any more element or fiber geometries would be obvious to a person of ordinary skill in the art. All of them are included in this invention.

### (e) Composition and Properties of Solid Core

Generally, in the invention herein, a solid core (e.g., a fluid-absorptive solid core, element, fiber, and so on) may comprise at least a physiological fluid-absorptive first excipient, also referred to herein as “fluid-absorptive first excipient”, “absorptive first excipient”, or “first excipient”. Generally, a solid core comprising an absorptive first excipient may expand with fluid absorption upon immersing said solid core in a physiological fluid under physiological conditions.

In some embodiments, therefore, upon exposure to or immersion in a physiological fluid under physiological conditions, an absorptive first excipient (or the excipient in the solid core in its totality) can absorb said physiological fluid and form solutions or mixtures with said fluid having a weight fraction of said physiological fluid greater than 0.2. This includes, but is not limited to an absorptive first excipient forming solutions or mixtures with a physiological fluid under physiological conditions having a weight fraction of said physiological fluid greater than 0.3, or greater than 0.4, or greater than 0.5, or greater than 0.6, or greater than 0.7, or greater than 0.8, or greater than 0.9, or greater than 0.95.

In some embodiments, moreover, the solubility of gastric fluid or a relevant physiological fluid in an absorptive first excipient under physiological conditions can be greater than about 200 mg/ml. This includes, but is not limited to solubility of gastric or relevant physiological fluid in an absorptive first excipient greater than 300 mg/ml, or greater than 400 mg/ml, or greater than 500 mg/ml, or greater than 600 mg/ml, or greater than 700 mg/ml, or greater than 750 mg/ml, or greater than 800 mg/ml, or greater than 850 mg/ml. Generally, a greater solubility of gastric or a relevant physiological fluid in an absorptive first excipient under physiological conditions may be preferable.

In some embodiments, moreover an effective diffusivity of physiological/body fluid in an absorptive first excipient (and/or an element or a segment comprising an absorptive first excipient) may be greater than 0.05×10⁻¹¹ m²/s under physiological conditions. This includes, but is not limited to an effective diffusivity of physiological/body fluid in an absorptive first excipient (and/or an element or a segment comprising an absorptive first excipient) greater than 0.1×10⁻¹¹ m²/s, or greater than 0.2×10⁻¹¹ m²/s, or greater than 0.5×10⁻¹¹ m²/s, or greater than 0.75×10⁻¹¹ m²/s, or greater than 1×10⁻¹¹ m²/s, or greater than 2×10⁻¹¹ m²/s, or greater than 3×10⁻¹¹ m²/s, or greater than 4×10⁻¹¹ m²/s under physiological conditions. Generally, a greater diffusivity of gastric or a relevant physiological fluid in an absorptive first excipient under physiological conditions may be preferable.

Alternatively, for absorptive first excipients where diffusion of physiological/body fluid to the interior may or may not be Fickian, a rate of penetration may be specified. In some embodiments, the rate of penetration of a physiological/body fluid into an element or a segment comprising an absorptive first excipient may be greater than an average thickness of said element divided by 3600 seconds (i.e., h₀/3600 μm/s). In other examples without limitation, rate of penetration may be greater than h₀/1800 μm/s, greater than h₀/1200 μm/s, greater than h₀/800 μm/s, greater than h₀/600 μm/s, or greater than h₀/500 μm/s, or greater than h₀/400 μm/s, or greater than h₀/300 μm/s. Generally, a greater rate of penetration of gastric or a relevant physiological fluid into an element comprising an absorptive first excipient under physiological conditions may be preferable.

For determining the effective diffusivity (and/or the rate of penetration) of dissolution medium in a solid, absorptive first excipient (and/or an element or a segment comprising said absorptive first excipient) the following procedure may be applied. An element (e.g an element or segment of the dosage form structure, or preferably an element or segment that just consists of the absorptive first excipient) may be placed in a still dissolution medium at 37° C. The time t₁ for the element to break apart or deform substantially may be recorded. (By way of example but not by way of limitation, a deformation of an element may generally be considered substantial if either the length, width, or thickness of the element differs by at least 20 to 80 percent (e.g., at least 20 percent, or at least 30 percent, or at least 40 percent, or at least 50 percent, or at least 60 percent, or at least 70 percent, or at least 80 percent, etc.) from its initial value.) The effective diffusivity, D_{eff}, may then be determined according to D_{eff}=hᵢₙᵢₜ²/4t₁ where hᵢₙᵢₜ is the initial element or segment thickness (e.g., the thickness of the dry element or segment). Similarly, the rate of penetration of a physiological/body fluid into the element or segment may be equal to hᵢₙᵢₜ/2t₁. Further non-limiting examples for deriving the effective diffusivity or rate of penetration are presented in U.S. application Ser. No. 15/482,776 titled “Fibrous dosage form”.

In some embodiments, at least one fluid-absorptive first excipient may comprise hydroxypropyl methylcellulose.

In some embodiments, at least one fluid-absorptive first excipient comprises hydroxypropyl methylcellulose with an average molecular weight greater than 30 kg/mol. In some embodiments, moreover, at least one fluid-absorptive first excipient comprises hydroxypropyl methylcellulose with a number-average molecular weight greater than 30 kg/mol.

In some embodiments, at least one fluid-absorptive first excipient may comprise hydroxypropyl methylcellulose with an average molecular weight greater than 30 kg/mol, and wherein the volume or weight fraction of hydroxypropyl methylcellulose with average molecular weight greater than 30 kg/mol in the fluid-absorptive solid core may be greater than 0.05.

In some embodiments, moreover, at least one fluid-absorptive first excipient may be selected from the group comprising hydroxypropyl methylcellulose, hydroxyethyl cellulose, polyvinyl alcohol, polyvinylpyrrolidone, sodium alginate, hydroxypropyl cellulose, hydroxyethyl cellulose, methyl cellulose, hydroxypropyl methyl ether cellulose, starch, chitosan, pectin, polymethacrylates (e.g., poly(methacrylic acid, ethyl acrylate) 1:1, or butylmethacrylat-(2-dimethylaminoethyl)methacrylat-methylmathacrylat-copolymer), polyacrylic acid, polyethylene oxide, vinylpyrrolidone-vinyl acetate copolymer, and/or others.

To ensure that a dosage form or drug releasable solid may expand substantially, and that the integrity of an expanded dosage form or an expanded expandable, drug releasable solid (e.g., a semi-solid or viscoelastic mass, etc.) may be preserved for prolonged time within a physiological fluid under physiological conditions, the molecular weight of the one or more physiological fluid-absorptive first excipients may be quite large. In some embodiments, therefore, the molecular weight (e.g., the number-average molecular weight) of at least one absorptive first excipient may be greater than 30 kg/mol. This includes, but is not limited to at least one fluid-absorptive first excipient having a molecular weight (e.g., a number-average molecular weight) greater than 40 kg/mol, or greater than 50 kg/mol, or greater than 60 kg/mol, or greater than 70 kg/mol, or greater than 80 kg/mol.

To ensure that the dosage form can be processed by patterning a viscous paste comprising absorptive first excipient, and for other reasons, the molecular weight of at least one absorptive first excipient (or the absorptive first excipient in its totality) may, however, also be limited.

By way of example but not by way of limitation, the number-average molecular weight of at least one absorptive first excipient (or the number-average molecular weight of the absorptive first excipient in its totality) may be in the ranges 30 kg/mol-10,000,000 kg/mol, 50 kg/mol-10,000,000 kg/mol, 70 kg/mol-10,000,000 kg/mol, 80 kg/mol-10,000,000 kg/mol, 70 kg/mol-5,000,000 kg/mol, 70 kg/mol-2,000,000 kg/mol. In preferable embodiments, a physiological fluid-absorptive first excipient comprises hydroxypropyl methylcellulose with a molecular weight (e.g., a number-average molecular weight) in the range between about 50 kg/mol and 1000 kg/mol (e.g., 70 kg/mol-500,000 kg/mol).

Thus, in some embodiments, at least one absorptive first excipient (or the absorptive excipient in its totality) may comprise a plurality of individual chains or molecules that dissolve or disentangle upon immersion in a physiological fluid.

In some embodiments, moreover, at least one absorptive first excipient may have a solubility greater than 20 g/l in a relevant physiological/body fluid under physiological conditions. This includes, but is not limited to at least one absorptive first excipient (or the absorptive first excipient in its totality) having a solubility in a relevant physiological/body fluid under physiological conditions greater than 50 g/l, or greater than 75 g/l, or greater than 100 g/l, or greater than 150 g/l, or greater than 175 g/l, or greater than 200 g/l, or greater than 250 g/l, or greater than 300 g/l, or greater than 350 g/l. In the extreme case, absorptive first excipient (e.g., at least one absorptive first excipient or the absorptive first excipient in its totality) may be mutually soluble with a relevant physiological fluid under physiological conditions. The solubility of a material is referred to herein as the maximum amount or mass of said material that can be dissolved at equilibrium in a given volume of physiological fluid under physiological conditions divided by the volume of said fluid or of the solution formed. By way of example but not by way of limitation, the solubility of a solute in a solvent may be determined by optical methods.

Preferably, moreover, at least one absorptive first excipient (or the absorptive first excipient in its totality) may comprise an amorphous molecular structure (e.g., an amorphous arrangement of molecules, or an arrangement of molecules without long-range order) in the solid state. A non-limiting method for determining the molecular structure of a solid (e.g., distinguishing amorphous molecular structure from crystalline molecular structure, etc.) is Differential Scanning Calorimetry.

In some embodiments, volume or weight fraction of one or more fluid-absorptive first excipients in a fluid-absorptive solid core may be greater than 0.05. This includes, but is not limited to volume of weight fraction of one or more fluid-absorptive first excipients greater than 0.15 or greater than 0.2.

In some embodiments the weight fraction of absorptive first excipient in at least a solid core (e.g., one or more elements, etc.) with respect to the total weight of said solid core may be greater than 0.1. This includes, but is not limited to a weight fraction of absorptive first excipient in a solid core with respect to the total weight of said solid core greater than 0.15, or greater than 0.2, or greater than 0.25, or greater than 0.3, or greater than 0.35, or greater than 0.4. Generally, a greater weight fraction of fluid-absorptive first excipient in a solid core may be preferable for achieving greater expansion of said solid core.

Similarly, in some embodiments the weight fraction of absorptive first excipient in a three dimensional structural framework of one or more elements (e.g., fibers) with respect to the total weight of said framework may be greater than 0.1. This includes, but is not limited to a weight fraction of absorptive first excipient in the structural framework with respect to the total weight of said framework greater than 0.15, or greater than 0.2, or greater than 0.25, or greater than 0.3, or greater than 0.35, or greater than 0.4. Generally, a greater weight fraction of fluid-absorptive first excipient in a framework may be preferable for achieving greater expansion of said framework.

In some embodiments, the concentration of at least an absorptive first excipient may be substantially uniform within or through or across a solid core (e.g., one or more elements, a three dimensional structural framework of elements, etc.).

In some embodiments, moreover, a solid core (e.g., one or more elements, etc.) may comprise a plurality of (e.g., two or more) segments having substantially the same weight fraction of physiological fluid-absorptive first excipient distributed within the segments (e.g., the standard deviation of the weight fraction of absorptive first excipient within the segments is no greater than the average value, etc.). In some embodiments, moreover, an element or framework may comprise a plurality of segments having substantially the same weight fraction of physiological fluid-absorptive first excipient distributed within the segments.

To stabilize a solid core, drug releasable solid, or dosage form after soaking with a physiological fluid under physiological conditions, in some embodiments, a fluid-absorptive solid core (e.g., an element, fiber, etc.) may further comprise a mechanically strengthening first excipient. A non-limiting example of a mechanically strengthening first excipient is methacrylic acid-ethyl acrylate copolymer.

Moreover, it may be obvious to a person of ordinary skill in the art that additional excipients, such as gastrointestinal contrast agents, fillers, process agents, and so on can be added to a fluid absorptive solid core.

Any further compositions of solid cores or physiological fluid-absorptive solid cores be obvious to a person of ordinary skill in the art. All of them are within the spirit and scope of this invention.

### (f) Microstructure of Mechanically Strengthening Layer-Encapsulated Solid Core and Mechanically Strengthening Layer-Encapsulated Three Dimensional Structural Framework of Elements or Fibers

Generally, for assuring that the dosage form possesses the desired expansion rate, mechanical properties, drug release rate, and so on, a mechanically strengthening semi-permeable layer as disclosed herein may substantially encapsulate a fluid-absorptive solid core or structural framework. Generally a core or framework may be referred to as “substantially encapsulated” by a mechanically strengthening semi-permeable layer if after expansion with fluid absorption the mechanical properties (e.g., the tensile strength, elastic modulus, etc.) of said expanded encapsulated core or framework are substantially greater than the mechanical properties of the expanded core or framework without encapsulation.

In some embodiments, moreover, a core or framework may be referred to as “substantially encapsulated” by a mechanically strengthening semi-permeable layer if said mechanically strengthening semi-permeable layer covers (e.g., encloses, coats, etc.) at least 20 percent of the surface of said core. This includes, but is not limited to said mechanically strengthening semi-permeable layer covering at least 30 percent, or at least 40 percent, or at least 50 percent, or at least 60 percent, or at least 70 percent, or at least 80 percent, or at least 90 percent, or about 100 percent of the surface of said core. Generally, a greater area of a solid core or framework covered by mechanically strengthening semi-permeable layer may be preferable for achieving a pharmaceutical solid dosage form with the desired properties.

It may generally be desirable that mechanically strengthening semi-permeable layer may substantially stabilize a large part or the entire body of an expanded solid or semi-solid. In preferred embodiments, therefore, mechanically strengthening phase may be substantially connected (e.g., substantially continuous, etc.) through a length, and/or through a width, and/or through a thickness, and/or through a volume of an encapsulated solid core (e.g., a solid core or framework encapsulated by said mechanically strengthening semi-permeable layer). In some embodiments, therefore, a mechanically strengthening, semi-permeable layer may form a substantially connected structure through or across an exterior volume of an encapsulated three dimensional structural framework of elements.

In some embodiments, moreover, a mechanically strengthening semi-permeable layer comprising a thickness or an average thickness greater than 1 μm (e.g., greater than 2 μm, no less than 5 μm, no less than 10 μm) may be substantially connected through a length of an encapsulated solid core (e.g., a solid core or framework encapsulated by said mechanically strengthening semi-permeable layer). This includes, but is not limited to a mechanically strengthening semi-permeable layer comprising a thickness or an average thickness greater than 1 μm (e.g., greater than 2 μm, no less than 5 μm, no less than 10 μm) substantially connected through a volume of an encapsulated solid core (e.g., a solid core or framework encapsulated by said mechanically strengthening semi-permeable layer).

Moreover as the theoretical models and the experimental examples suggest, a relevant parameter for determining the expansion rate and other properties of a dosage form (or of an encapsulated fluid-absorptive core or framework, or of an expandable, drug releasable solid) herein may be the volume fraction of mechanically strengthening, semi-permeable layer in an outer volume of an encapsulated solid core or three dimensional structural framework. In some embodiments, a larger volume fraction of mechanically strengthening semi-permeable layer may decrease the expansion rate of a dosage form (or of an encapsulated fluid-absorptive, expandable solid core or of an expandable, drug releasable solid) but enhance several mechanical properties, such as a tensile strength, an elastic modulus, etc., of an expanded dosage form or an expanded drug releasable solid.

In some embodiments, the volume fraction of mechanically strengthening semi-permeable layer within an outer volume of a three dimensional structural framework encapsulated by said mechanically strengthening semi-permeable layer may be no less than 0.005. This includes, but is not limited to a volume fraction of mechanically strengthening layer within an outer volume of a three dimensional structural framework encapsulated by said mechanically strengthening layer no less than 0.01, or no less than 0.02, or no less than 0.03, or greater than 0.03, or greater than 0.04, or greater than 0.05, or greater than 0.06, or greater than 0.07, or greater than 0.08, or greater than 0.09, or greater than 0.1.

The volume fraction of mechanically strengthening semi-permeable layer within an outer volume of a three dimensional structural framework encapsulated by said mechanically strengthening semi-permeable layer may, however, also not be too large to assure that the dosage form or drug releasable solid possesses the desired properties. In some embodiments, therefore, the volume fraction of mechanically strengthening semi-permeable layer within an outer volume of a three dimensional structural framework encapsulated by said mechanically strengthening semi-permeable layer may be in the range between 0.005 and 0.7. This includes, but is not limited to a volume fraction of mechanically strengthening layer within an outer volume of a three dimensional structural framework encapsulated by said mechanically strengthening layer in the ranges 0.01-0.7, or 0.02-0.7, or 0.005-0.65, or 0.01-0.65, or 0.02-0.65.

In some embodiments, moreover, the weight fraction of mechanically strengthening semi-permeable layer within an outer volume of a three dimensional structural framework encapsulated by said mechanically strengthening semi-permeable layer may be no less than 0.005. This includes, but is not limited to a weight fraction of mechanically strengthening layer within an outer volume of a three dimensional structural framework encapsulated by said mechanically strengthening layer no less than 0.01, or no less than 0.02, or no less than 0.03, or greater than 0.03, or greater than 0.04, or greater than 0.05, or greater than 0.06, or greater than 0.07, or greater than 0.08, or greater than 0.09, or greater than 0.1.

In some embodiments, moreover, the weight fraction of mechanically strengthening semi-permeable layer within an outer volume of a three dimensional structural framework encapsulated by said mechanically strengthening semi-permeable layer may be in the range between 0.005 and 0.7. This includes, but is not limited to a weight fraction of mechanically strengthening layer within an outer volume of a three dimensional structural framework encapsulated by said mechanically strengthening layer in the ranges 0.01-0.7, or 0.02-0.7, or 0.005-0.65, or 0.01-0.65, or 0.02-0.65, or 0.01-0.55, or 0.01-0.5, or 0.01-0.45, or 0.01-0.4.

In some embodiments, moreover the volume of mechanically strengthening semi-permeable surface layer per unit volume of an exterior volume of an encapsulated solid core (e.g., a solid core encapsulated by said mechanically strengthening semi-permeable surface layer) may be greater than 0.005. This includes, but is not limited to volume of mechanically strengthening semi-permeable surface layer per unit volume of an exterior volume of an encapsulated solid core greater than 0.01, or greater than 0.015, or greater than 0.02, or greater than 0.025.

In some embodiments, the weight of mechanically strengthening semi-permeable surface layer per unit volume of an exterior volume of an encapsulated solid core (e.g., the density of said mechanically strengthening semi-permeable surface layer in said encapsulated solid core) is greater than 5 kg/m³. This includes, but is not limited to a weight of mechanically strengthening semi-permeable surface layer per unit volume of an exterior volume of an encapsulated solid core greater than 10 kg/m³, or greater than 15 kg/m³, or greater than 20 kg/m³.

In some embodiments, moreover, the ratio of the volume fraction of a fluid-absorptive three dimensional structural framework and the volume fraction of mechanically strengthening, semi-permeable layer within an outer volume of an encapsulated three dimensional structural framework may be in the range between 0.1 and 50. This includes, but is not limited to a ratio of the volume fraction of a fluid-absorptive three dimensional structural framework and the volume fraction of mechanically strengthening, semi-permeable layer within an outer volume of an encapsulated three dimensional structural framework is in the ranges 0.1-40, or 0.2-0.50, or 0.2-40, or 0.1-30, or 0.2-30.

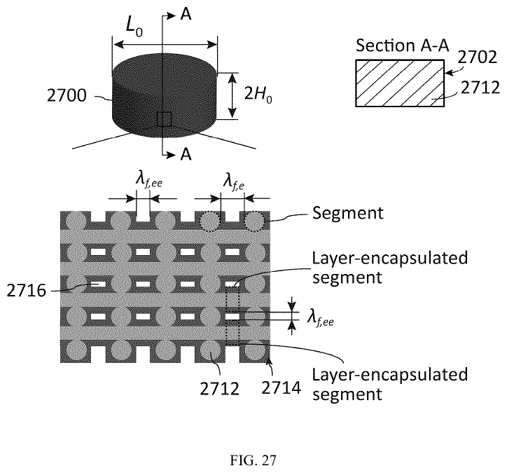

FIG. 27 presents a non-limiting dosage form 2700 comprising a fluid-absorptive solid core 2712 and a mechanically strengthening, semi-permeable layer 2714. The fluid-absorptive solid core 2712 comprises a three-dimensional structural framework of structural elements 2712. The elements 2712 are substantially encapsulated (e.g., substantially coated, substantially surrounded, etc.) by said mechanically strengthening, semi-permeable layer 2714. The surface layer-encapsulated elements 2712, 2714 (e.g., the elements 2712 and the encapsulating layer 2714 combined) further comprise surface layer-encapsulated segments spaced apart from adjoining surface layer-encapsulated segments by encapsulated-element-free spacings, λ_{f,ee}, thereby defining one or more encapsulated-element-free spaces 2716 in or through the exterior volume of the encapsulated solid core 2712 or framework 2712.

As for the three dimensional structural framework of elements, in some embodiments the encapsulated three dimensional structural framework of elements may comprise one or more substantially connected encapsulated-element-free spaces within its exterior volume.

In some embodiments of the invention herein, one or more encapsulated-element-free spaces within an exterior volume of an encapsulated three dimensional structural framework of elements may be considered “substantially connected” if a fluid can substantially percolate said one or more encapsulated-element-free spaces upon immersing said encapsulated framework in said fluid.

In some embodiments of the invention herein, moreover, one or more encapsulated-element-free spaces within an exterior volume of an encapsulated three dimensional structural framework of elements may be considered “substantially connected” if they extend over a length at least about a third of an exterior dimension of said encapsulated structural framework. This includes, but is not limited to one or more encapsulated-element-free spaces within an exterior volume of an encapsulated three-dimensional structural framework of elements extending over a length at least two thirds of an exterior dimension of said encapsulated structural framework, or over a length at least equal to an exterior dimension of said encapsulated structural framework, or over a length and width at least equal to an exterior dimension of said encapsulated structural framework, or over the entire length, width, and thickness of the exterior volume of said encapsulated three-dimensional structural framework.

In the invention herein, one or more encapsulated-element-free spaces that are “connected” may also be referred to as one or more encapsulated-element-free spaces that are “interconnected”, “contiguous”, “in direct contact”, “merged”, “without any wall in between”, and so on. Similarly, an “exterior dimension of a encapsulated structural framework” may be considered a “length of an exterior volume of said encapsulated structural framework”, and so on.

Any more microstructures of surface layer-encapsulated solid core and surface layer-encapsulated three dimensional structural framework of elements would be obvious to a person of ordinary skill in the art. All of them are included in this invention.

### (g) Encapsulated-Element-Free Spacing

As for the element-free spacing, to assure that fluid can percolate encapsulated-element-free space and/or to assure that encapsulated-element-free space can be adequately occupied by drug-containing solid and/or free space into which a physiological fluid can percolate, the encapsulated-element-free spacing (e.g., the channel size or diameter, channel width, pore size, etc.) between encapsulated elements, encapsulated fibers, or encapsulated segments may be on the micro- or macro-scale.

Thus, in some embodiments, an encapsulated-element-free spacing, λ_{f,ee}, between encapsulated elements (e.g., encapsulated fibers, etc.) or encapsulated segments through (e.g., across, along, etc.) one or more encapsulated element-free spaces or one or more connected encapsulated-element-free spaces may be greater than 10 μm. This includes, but is not limited to λ_{f,ee} greater than 15 μm, or greater than 20 μm, or greater than 25 μm, or greater than 30 μm, or greater than 35 μm, or greater than 40 μm, or greater than 45 μm, or greater than 50 μm, or greater than 60 μm, or greater than 70 μm, or greater than 80 μm, or greater than 90 μm, or greater than 100 μm, or greater than 125 μm, or greater than 150 μm, or greater than 175 μm, or greater than 200 μm.

The encapsulated-element-free spacing may, however, also not be too large. Thus, in some embodiments, the encapsulated-element-free spacing through (e.g., along, across, etc.) one or more encapsulated-element-free spaces or through (e.g., along, across, etc.) a connected encapsulated-element-free space may be in the ranges 10 μm-5 mm, 10 μm-3 mm, 15 μm-5 mm, 25 μm-5 mm, 25 μm-3 mm, 50 μm-4.5 mm, 50 μm-4 mm, 100 μm-5 mm, 100 μm-4 mm, 200 μm-5 mm, 150 μm-4 mm, 200 μm-4 mm, 50 μm-4 mm, 40 μm-4 mm, or 100 μm-5 mm.

The encapsulated-element-free spacing may be determined experimentally from microstructural images (e.g., scanning electron micrographs, micro computed tomography scans, and so on) of an encapsulated solid framework or of an expandable, drug releasable solid. Non-limiting examples describing and illustrating how an encapsulated-element-free spacing may be determined from microstructural images are given in the U.S. application Ser. No. 15/482,776 titled “Fibrous dosage form”.

It may be noted, that in some embodiments herein the encapsulated-element-free spacing between encapsulated elements or encapsulated segments within an exterior volume of an encapsulated three dimensional structural framework or through (e.g., along, across, etc.) one or more encapsulated-element-free spaces may be precisely controlled.

Any more details of encapsulated-element-free spacings would be obvious to a person of ordinary skill in the art. All of them are included in this invention.

### (h) Composition and Properties of Mechanically Strengthening Semi-Permeable Layer

Generally, a mechanically strengthening semi-permeable layer comprises at least a mechanically strengthening second excipient. A mechanically strengthening second excipient is also referred to herein as “strength-enhancing second excipient”, “strengthening second excipient” or “second excipient”.

For assuring that a pharmaceutical dosage form or drug releasable solid possesses desirable properties, a mechanically strengthening semi-permeable layer (or a strength-enhancing second excipient, or strength-enhancing second excipient in its totality, etc.) may have several characteristics, features, or properties.

By way of example but not by way of limitation, for assuring that a mechanically strengthening semi-permeable layer-encapsulated solid core can expand with fluid absorption, a mechanically strengthening semi-permeable layer (or a strengthening second excipient, or strengthening second excipient in its totality, etc.) may form a viscoelastic, semi-permeable membrane or material upon exposure to a physiological fluid (e.g., gastric fluid) under physiological conditions.

Similarly, for assuring that a mechanically strengthening semi-permeable layer-encapsulated solid core can expand with fluid absorption, a mechanically strengthening semi-permeable layer (or a strengthening second excipient, or strengthening second excipient in its totality, etc.) that encapsulates a fluid-absorptive solid core may be permeable or substantially permeable to a physiological fluid upon immersing (e.g. upon soaking, etc.) said mechanically strengthening semi-permeable layer-encapsulated solid core in a physiological fluid under physiological conditions.

In some embodiments, therefore, the diffusivity of a relevant physiological fluid under physiological conditions in at least a mechanically strengthening layer (or a strengthening second excipient, or strengthening second excipient in its totality, etc.) may be greater than 0.5×10⁻¹³ m²/s. This includes, but is not limited to a diffusivity of a relevant physiological fluid under physiological conditions in at least a mechanically strengthening layer (or in at least one strengthening second excipient, or in the strengthening second excipient in its totality) greater than 1×10⁻¹³ m²/s, or greater than 2×10⁻¹³ m²/s, or greater than 5×10⁻¹³ m²/s, or greater than 1×10⁻¹² m²/s, or greater than 2×10⁻¹² m²/s, or greater than 3×10⁻¹² m²/s, or greater than 4×10⁻¹² m²/s, or greater than 5×10⁻¹² m²/s, or greater than 6×10⁻¹² m²/s, or greater than 1×10⁻¹¹ m²/s. A larger fluid diffusivity is generally preferable for promoting rapid expansion of the solid core.

Similarly, for preventing or reducing outflow of a fluid-absorptive excipient (e.g., a fluid-absorptive first excipient) from a mechanically strengthening semi-permeable layer-encapsulated solid core, a mechanically strengthening semi-permeable layer that encapsulates a fluid-absorptive solid core may be substantially impermeable to a fluid-absorptive first excipient upon immersing (e.g., soaking, etc.) said mechanically strengthening semi-permeable layer-encapsulated solid core in a physiological fluid under physiological conditions.

In some embodiments, accordingly, upon immersing a mechanically strengthening semi-permeable layer-encapsulated solid core (e.g., enclosing or encapsulating layer and solid core combined) in a relevant physiological fluid under physiological conditions, the diffusivity of at least one physiological fluid-absorptive first excipient in or through said mechanically strengthening, semi-permeable layer (or through a strengthening second excipient, or through a strengthening second excipient in its totality, etc.) may be no greater than 1×10⁻¹¹ m²/s. This includes, but is not limited to a diffusivity of at least one physiological fluid-absorptive first excipient in or through a mechanically strengthening, semi-permeable layer (or through a strengthening second excipient or through a strengthening second excipient in its totality) after soaking with a physiological fluid under physiological conditions no greater than 5×10⁻¹² m²/s, or no greater than 2×10⁻¹² m²/s, or no greater than 1×10⁻¹² m²/s, or no greater than 5×10⁻¹³ m²/s, or no greater than 2×10⁻¹³ m²/s, or no greater than 1×10⁻¹³ m²/s, or no greater than 5×10⁻¹⁴ m²/s, or no greater than 2×10⁻¹⁴ m²/s. Generally, a smaller diffusivity of absorptive first excipient through a mechanically strengthening layer after soaking with a physiological fluid under physiological conditions may be preferable for preserving the integrity of an expanded dosage form (or of an expanded mechanically strengthening semi-permeable layer-encapsulated solid core, or of an expanded expandable, drug releasable solid) in a physiological fluid.

In some embodiments, furthermore, upon immersing a mechanically strengthening semi-permeable layer-encapsulated solid core (e.g., enclosing or encapsulating layer and solid core combined) in a relevant physiological fluid under physiological conditions, the diffusivity of at least one physiological fluid-absorptive first excipient in or through said mechanically strengthening, semi-permeable layer (or through a strength-enhancing second excipient, or through strength-enhancing second excipient in its totality, etc.) is no greater than 0.3 times the self-diffusivity of said at least one absorptive first excipient in a relevant physiological fluid under physiological conditions. This includes, but is not limited to the diffusivity of at least one absorptive first excipient in or through mechanically strengthening, semi-permeable surface layer (or through a strength-enhancing second excipient, or through strength-enhancing second excipient in its totality, etc.) after soaking with a physiological fluid under physiological conditions no greater than 0.2 times, or no greater than 0.1 times, or no greater than 0.05 times, or no greater than 0.02 times, or no greater than 0.01 times, or no greater than 0.005 times, or no greater than 0.002 times, or no greater than 0.001 times the self-diffusivity of said at least one absorptive first excipient in a relevant physiological fluid under physiological conditions.

Generally, to assure that a mechanically strengthening, semi-permeable layer (e.g., mechanically strengthening second excipient) remains a semi-solid or viscoelastic material and stabilizes, or mechanically supports or enforces a core (e.g., one or more elements) after exposure to a physiological fluid (e.g., gastric fluid, etc.), the solubility of said mechanically strengthening, semi-permeable layer (e.g., said strengthening second excipient) in said physiological fluid may be limited. In some embodiments, therefore, at least one mechanically strengthening second excipient may have a solubility no greater than 1 g/l in a relevant physiological/body fluid under physiological conditions. This includes, but is not limited to at least one mechanically strengthening second excipient (or one or more strengthening second excipients, or the strengthening second excipient in its totality) having a solubility in a relevant physiological/body fluid under physiological conditions no greater than 0.5 g/l, or no greater than 0.4 g/l, or no greater than 0.2 g/l, or no greater than 0.1 g/l, or no greater than 0.05 g/l, or no greater than 0.02 g/l, or no greater than 0.01 g/l, or no greater than 0.005 g/l, or no greater than 0.002 g/l, or no greater than 0.001 g/l. In the extreme case, strengthening second excipient (e.g., at least one strengthening second excipient or the strengthening second excipient in its totality) may be insoluble or at least practically insoluble in a relevant physiological fluid under physiological conditions. A smaller solubility of mechanically strengthening, semi-permeable layer (or a strengthening second excipient) in physiological fluid may generally be preferable for preserving the integrity of an expanded dosage form (or of an expanded mechanically strengthening semi-permeable layer-encapsulated solid core, or of an expanded expandable, drug releasable solid) in a physiological fluid.

It may be noted that even if the solubility of a relevant physiological fluid is low in a mechanically strengthening, semi-permeable layer (or in a strengthening second excipient, or in strengthening second excipient in its totality, etc.), said mechanically strengthening, semi-permeable layer (e.g., said strengthening second excipient, or said strengthening second excipient in its totality, etc.) may soften or plasticize somewhat upon contact with or immersion in said physiological fluid under physiological conditions. As a result, a mechanically strengthening, semi-permeable layer (e.g., at least a strengthening second excipient) can be a solid in the dry state, but upon immersion in or exposure to a relevant physiological fluid (e.g., gastric fluid, etc.) under physiological conditions, it may transition to a semi-solid or viscoelastic material.

Generally, the mechanical properties (such as stiffness, yield strength, tensile strength, elongational viscosity, etc.) of physiological fluid-soaked mechanically strengthening, semi-permeable layer (e.g. physiological fluid-soaked strengthening second excipient, physiological fluid-soaked strengthening second excipient in its totality, etc.), should be large enough to stabilize or mechanically support the dosage form, or an expandable, drug releasable solid, or an encapsulated structural framework of elements after soaking with a physiological fluid under physiological conditions. In the invention herein, the term “physiological fluid-soaked mechanically strengthening, semi-permeable layer” is generally referred to as a film of mechanically strengthening, semi-permeable layer that is/has been immersed in a relevant physiological fluid (e.g., acidic water) for so long that the water concentration in the film may be roughly at equilibrium.

However, the stiffness, yield strength, tensile strength, elongational viscosity, etc. of physiological fluid-soaked mechanically strengthening, semi-permeable layer should not be too large, so that the expansion of the dosage form or drug releasable solid or structural framework after exposure to said physiological fluid may not be excessively impaired or constrained. Thus, mechanically strengthening, semi-permeable layers (e.g., strengthening second excipients) that comprise or form a viscoelastic or semi-solid material upon exposure to a relevant physiological fluid are typically preferred herein.

In some embodiments, physiological fluid-soaked mechanically strengthening, semi-permeable layer (e.g., physiological fluid-soaked strengthening second excipient, physiological fluid-soaked strengthening second excipient in its totality, etc.) may comprise an elastic modulus, or an elastic-plastic modulus, or a plastic modulus greater than 0.02 MPa. This includes, but is not limited to physiological fluid-soaked mechanically strengthening, semi-permeable layer (e.g., physiological fluid-soaked strengthening second excipient, physiological fluid-soaked strengthening second excipient in its totality, etc.) comprising an elastic modulus, or an elastic-plastic modulus, or a plastic modulus greater than 0.05 MPa, or greater than 0.1 MPa, or greater than 0.2 MPa, or greater than 0.3 MPa, or greater than 0.4 MPa, or greater than 0.5 MPa, or greater than 0.6 MPa, or greater than 0.7 MPa, or greater than 0.8 MPa, or greater than 0.9 MPa, or greater than 1 MPa.

In some embodiments, moreover, physiological fluid-soaked mechanically strengthening, semi-permeable layer (e.g., physiological fluid-soaked strengthening second excipient, physiological fluid-soaked strengthening second excipient in its totality, etc.) may comprise an elastic modulus, or an elastic-plastic modulus, or a plastic modulus no greater than about 1000 MPa (e.g., no greater than 500 MPa, or no greater than 200 MPa, or no greater than 100 MPa, or no greater than 50 MPa, or no greater than 20 MPa, or no greater than 10 MPa). Preferably, an elastic modulus of a physiological fluid-soaked strengthening second excipient may be no less than about 0.1 MPa and no greater than about 100 MPa.

In some embodiments, moreover, physiological fluid-soaked mechanically strengthening, semi-permeable layer (e.g., physiological fluid-soaked strengthening second excipient, physiological fluid-soaked strengthening second excipient in its totality, etc.) may comprise a yield strength greater than 0.005 MPa. This includes, but is not limited to physiological fluid-soaked mechanically strengthening, semi-permeable layer (e.g., physiological fluid-soaked strengthening second excipient, physiological fluid-soaked strengthening second excipient in its totality, etc.) comprising a yield strength greater than 0.0075 MPa, or greater than 0.01 MPa, or greater than 0.02 MPa, or greater than 0.05 MPa, or greater than 0.1 MPa, or greater than 0.2 MPa. In some embodiments, moreover, physiological fluid-soaked mechanically strengthening, semi-permeable layer (e.g., physiological fluid-soaked strengthening second excipient, physiological fluid-soaked strengthening second excipient in its totality, etc.) comprises a yield strength no greater than 500 MPa (e.g., no greater than 200 MPa, or no greater than 100 MPa, or no greater than 75 MPa, or no greater than 50 MPa, or no greater than 20 MPa, or no greater than 10 MPa, or no greater than 5 MPa).

In some embodiments, moreover, physiological fluid-soaked mechanically strengthening, semi-permeable layer (e.g., physiological fluid-soaked strengthening second excipient, physiological fluid-soaked strengthening second excipient in its totality, etc.) may comprise a tensile strength (or a fracture strength) greater than 0.02 MPa. This includes, but is not limited to physiological fluid-soaked mechanically strengthening, semi-permeable layer (e.g., physiological fluid-soaked strengthening second excipient, physiological fluid-soaked strengthening second excipient in its totality, etc.) comprising a tensile strength (or a fracture strength) greater than 0.05 MPa, or greater than 0.08 MPa, or greater than 0.1 MPa, or greater than 0.2 MPa, or greater than 0.3 MPa, or greater than 0.4 MPa, or greater than 0.5 MPa, or greater than 0.6 MPa.

In some embodiments, moreover, physiological fluid-soaked mechanically strengthening, semi-permeable layer (e.g., physiological fluid-soaked strengthening second excipient, physiological fluid-soaked strengthening second excipient in its totality, etc.) may comprise a tensile strength (or a fracture strength) no greater than 500 MPa (e.g., no greater than 200 MPa, or no greater than 100 MPa, or no greater than 75 MPa, or no greater than 50 MPa, or no greater than 20 MPa, or no greater than 10 MPa).

In some embodiments, moreover, physiological fluid-soaked mechanically strengthening, semi-permeable layer (e.g., physiological fluid-soaked strengthening second excipient, physiological fluid-soaked strengthening second excipient in its totality, etc.) comprises a strain at fracture greater than 0.2. This includes, but is not limited to physiological fluid-soaked mechanically strengthening, semi-permeable layer (e.g., physiological fluid-soaked strength-enhancing second excipient, physiological fluid-soaked strength-enhancing second excipient in its totality, etc.) comprising a strain at fracture greater than 0.5, or greater than 0.75, or greater than 1, or greater than 1.25, or greater than 1.5, or greater than 1.75, or greater than 2, or greater than 2.25, or greater than 2.5. Preferably, the strain at fracture of a physiological fluid-soaked mechanically strengthening semi-permeable layer may be greater than about 1.

In some preferred embodiments, physiological fluid-soaked mechanically strengthening, semi-permeable layer (e.g., physiological fluid-soaked strengthening second excipient, physiological fluid-soaked strengthening second excipient in its totality, etc.) may be a viscoelastic material. If exposed to a (small) stress for a short time (e.g., for a time smaller than about the relaxation time), it may deform elastically and spring back. If exposed to a (small) stress for a long time (e.g., for a time longer or much longer than about the relaxation time), it may deform plastically.

In some embodiments, upon exposure to a stress for a long time (e.g., for a time longer or much longer than the relaxation time), physiological fluid-soaked mechanically strengthening, semi-permeable layer (e.g., physiological fluid-soaked strengthening second excipient, physiological fluid-soaked strengthening second excipient in its totality, etc.) may deform plastically and essentially behave like a viscous material having an elongational viscosity. In some embodiments, elongational viscosity of physiological fluid-soaked mechanically strengthening, semi-permeable layer (e.g., physiological fluid-soaked strengthening second excipient, physiological fluid-soaked strengthening second excipient in its totality, etc.) may be no greater than 1×10¹¹ Pa·s. This includes, but is not limited to elongational viscosity of physiological fluid-soaked mechanically strengthening, semi-permeable layer (e.g., physiological fluid-soaked strengthening second excipient, physiological fluid-soaked strengthening second excipient in its totality, etc.) no greater than 5×10¹⁰ Pa·s, or no greater than 2×10¹⁰ Pa·s, or no greater than 1×10¹⁰ Pa·s, or no greater than 5×10⁹ Pa·s, or no greater than 2×10⁹ Pa·s, or no greater than 1×10⁹ Pa·s.

In some embodiments, moreover, elongational viscosity of physiological fluid-soaked mechanically strengthening, semi-permeable layer (e.g., physiological fluid-soaked strengthening second excipient, physiological fluid-soaked strengthening second excipient in its totality, etc.) may be greater or no less than 1×10⁵ Pa·s. This includes, but is not limited to elongational viscosity of physiological fluid-soaked mechanically strengthening, semi-permeable layer (e.g., physiological fluid-soaked strengthening second excipient, physiological fluid-soaked strengthening second excipient in its totality, etc.) greater than 2×10⁵ Pa·s, or greater than 5×10⁵ Pa·s, or greater than 1×10⁶ Pa·s, or greater than 2×10⁶ Pa·s, or greater than 5×10⁶ Pa·s, or greater than 1×10⁷ Pa·s.

In some embodiments, moreover, elongational viscosity of physiological fluid-soaked mechanically strengthening, semi-permeable layer (e.g., physiological fluid-soaked strengthening second excipient, physiological fluid-soaked strengthening second excipient in its totality, etc.) may be in the range 1×10⁵ Pa·s-1×10¹¹ Pa·s, and more preferably in the range 5×10⁵ Pa·s-5×10¹⁰ Pa·s, and even more preferably in the range 1×10⁶ Pa·s-2×10¹⁰ Pa·s, and even more preferably in the range 2×10⁶ Pa·s-1×10¹⁰ Pa·s, which includes, but is not limited to elongational viscosity of physiological fluid-soaked mechanically strengthening, semi-permeable layer in the range 5×10⁶ Pa·s-5×10⁹ Pa·s.

Furthermore, in some embodiments, the solubility of at least a mechanically strengthening second excipient (or the solubility of mechanically strengthening second excipient in its totality) can differ in different physiological fluids under physiological conditions. By way of example but not by way of limitation, in some embodiments the solubility of at least one mechanically strengthening second excipient in aqueous physiological fluid may depend on the pH value of said physiological fluid. More specifically, in some embodiments at least one mechanically strengthening second excipient can be sparingly-soluble or insoluble or practically insoluble in an aqeuous physiological fluid that is acidic (e.g., in gastric fluid, or in fluid with a pH value smaller than about 4, or in fluid with a pH value smaller than about 5, etc.), but it can be soluble in an aqueous physiological fluid having a greater pH value (e.g., in a fluid with a pH value greater than about 6, or greater than about 6.5, or greater than about 7, or greater than about 7.5, etc.), such as intestinal fluid. A mechanically strengthening second excipient comprising a solubility that is smaller in acidic solutions than in pH neutral or basic solutions is also referred to herein as “enteric excipient”.

In some embodiments, therefore, at least one strengthening second excipient comprises a solubility in aqueous fluid with a pH value no greater than 4 at least 10 (e.g., at least 20, or at least 50, or at least 100, or at least 200, or at least 500) times smaller than the solubility of said strengthening second excipient in an aqueous fluid with a pH value greater than 7 (e.g., the latter includes, but is not limited to an aqueous fluid with a pH value greater than 8).

A non-limiting example of such a mechanically strengthening second excipient that is sparingly-soluble in gastric or acidic fluid, but dissolves in intestinal fluid (e.g., aqueous fluid with a pH value greater than about 5.5), is methacrylic acid-ethyl acrylate copolymer. In some embodiments, therefore, a strengthening second excipient comprises is methacrylic acid-ethyl acrylate copolymer.

Another non-limiting example of a mechanically strengthening second excipient is polyvinyl acetate.

Other non-limiting examples of strength-enhancing second excipients herein may include methacrylic acid-ethyl acrylate copolymer, methacrylic acid-methyl methacrylate copolymer, ethyl acrylate-methylmethacrylate copolymer, hydroxypropyl methylcellulose acetate succinate, polyvinyl acetate, polymers including methacrylic acid, polymers including ethyl acrylate, polymers including methyl methacrylate, polymers including methacrylate, Poly[Ethyl acrylate, methyl methacrylate, trimethylammonioethyl methacrylate chloride], and ethylcellulose, and so on.

Moreover, to assure that one or more strengthening second excipients (or strengthening second excipient in its totality) may mechanically strengthen an expanded encapsulated core adequately or sufficiently, in some embodiments a volume or weight fraction of one or more strengthening second excipients (or the strengthening second excipient in its totality) in a mechanically strengthening semi-permeable layer may be greater than 0.35. This includes, but is not limited to a volume or weight fraction of one or more strengthening second excipients (or the strengthening second excipient in its totality) in a mechanically strengthening semi-permeable layer greater than 0.4, or greater than 0.45, or greater than 0.5, or greater than 0.55, or greater than 0.6.

Any more examples, details, or features of a mechanically strengthening semi-permeable layer or of a strengthening second excipient obvious to a person of ordinary skill in the art are all included in the spirit and scope of this invention.

### (i) Structure of Drug-Containing Solid

In addition to the solid core and the mechanically strengthening semi-permeable layer, the pharmaceutical solid dosage form disclosed herein may comprise a drug-containing solid. Generally, at least a fraction (e.g., at least part) of said drug-containing solid may cover, encapsulate, surround, coat, etc. a mechanically strengthening semi-permeable layer or a layer-encapsulated solid core or structural framework. Such placement of the drug-containing solid may prevent that mechanically strengthening semi-permeable layer blocks or slows down drug release by the drug-containing solid upon immersing said pharmaceutical solid dosage form in a physiological fluid. Thus, such placement of the drug-containing solid may enable a greater range and improved control of the drug release rate by said drug-containing solid into said physiological fluid. It may be noted that a drug-containing solid that is attached to, bonded to, covers, encapsulates, surrounds, coats, etc. a mechanically strengthening semi-permeable layer may generally be understood as a drug-containing solid that may be attached to, bonded to, cover, encapsulate, surround, coat, etc. a surface of said mechanically strengthening semi-permeable layer.

In some embodiments, at least a fraction (e.g., at least part) of a drug-containing solid may be attached to, joined to, connected to, bonded to, immovably attached to, etc. a mechanically strengthening semi-permeable layer or a layer-encapsulated solid core or structural framework.

In some embodiments, moreover, a drug-containing solid that is attached to, joined to, connected to, bonded to, etc. a mechanically strengthening semi-permeable layer or a layer-encapsulated solid core or structural framework may remain connected, attached, immovably attached, bonded, etc. to said mechanically strengthening semi-permeable layer or layer-encapsulated solid core or structural framework upon immersing in a physiological fluid under physiological conditions. In such embodiments, if a layer-encapsulated solid core or structural framework remains in the stomach for prolonged time, a drug-containing solid attached to, immovably attached to, connected to, bonded to, joined to, etc. said layer-encapsulated solid core or structural framework may remain in the stomach for prolonged time, too, for releasing drug into gastric fluid over time.

In preferred embodiments, therefore, a drug-containing solid may be bonded to (e.g., bonded or attached to a surface of) a mechanically strengthening semi-permeable layer.

In preferred embodiments, moreover, a drug-containing solid may be bonded to (e.g., bonded or attached to a surface of) a mechanically strengthening semi-permeable layer by interdiffusion of molecules between drug-containing solid and mechanically strengthening semi-permeable layer or by welding drug-containing solid and mechanically strengthening semi-permeable layer together.

In some embodiments, moreover, a drug-containing solid may occupy one or more encapsulated fiber-free spaces within a pharmaceutical solid dosage form (or within an outer volume of an encapsulated three dimensional structural framework of elements or within an expandable, drug releasable solid). In some embodiments, moreover, one or more encapsulated fiber-free spaces within a pharmaceutical solid dosage form (or within an outer volume of an encapsulated three dimensional structural framework of elements or within an expandable, drug releasable solid) may be occupied by free space that is not occupied by said drug-containing solid. Said free space may enable fluid percolation into the pharmaceutical solid dosage form or drug releasable solid upon immersing said pharmaceutical solid dosage form or drug releasable solid in a physiological fluid under physiological conditions.

In some embodiments, therefore said drug-containing solid may form at least an annulus within one or more encapsulated element-free spaces or within one or more fiber-free spaces. The annulus may comprise a solid wall comprising the drug-containing solid.

For various reasons, including achieving faster expansion rate, improved control of the drug release rate, and so on, a thickness of a drug-containing solid may be controlled. In some embodiments, moreover, the thickness or average thickness of a drug-containing solid may be no greater than about 5 mm. This includes, but is not limited to a thickness or average thickness of a drug-containing solid no greater than 4.5 mm, or no greater than 4 mm, or no greater than 3.5 mm, or no greater than 3 mm, or no greater than 2.5 mm, or no greater than 2 mm, or no greater than 1.5 mm.

It may be noted, however, that if the drug-containing solid is too thin, it may be difficult to manufacture and hence the properties may again be difficult to control. Thus, in some embodiments, a drug-containing solid has a thickness or average thickness in the range of 5 μm-5 mm. This includes, but is not limited to thickness or average thickness of a drug-containing solid in the ranges 10 μm-5 mm, 20 μm-5 mm, 25 μm-4 mm, 30 μm-3 mm, 20 μm-3.5 mm, 25 μm-5 mm, 25 μm-2.5 mm, 25 μm-1.5 mm, 30 μm-2 mm, 30 μm-1.5 mm, 40 μm-3 mm, 10 μm-1.5 mm, or 50 μm-2.5 mm.

It may be noted, furthermore that the surface area of drug-containing solid may be altered for controlling the rate at which drug is released into a physiological fluid under physiological conditions. A large surface area may be desirable for achieving a large drug release rate, whereas a smaller surface area may be desirable for achieving a smaller drug release rate.

Thus, generally, a thickness (e.g., an average thickness) and surface area of a drug-containing solid may be so determined that the pharmaceutical solid dosage form or expandable, drug releasable solid comprises a desired amount or mass of active ingredient and releases said active ingredient at the desired rate over the desired time.

Any more embodiments and/or examples of the structure of a drug-containing solid obvious to a person of ordinary skill in the art are all within the spirit and scope of this invention.

### (j) Composition and Microstructure of Drug-Containing Solid

Generally, a drug-containing solid as disclosed herein includes at least an active ingredient or drug. In preferred embodiments, moreover, to control the rate by which a drug-containing solid releases drug and/or to control the time over which a drug-containing solid releases drug, a drug-containing solid may further comprise one or more third excipients.

Thus, in preferred embodiments a drug-containing solid may comprise at least a drug and one or more third excipients. In such embodiments one or more drug(s) may be dispersed as particles and/or as molecules in one or more third excipient(s). Thus, the one or more third excipient(s) may form an excipient matrix. The drug may be dispersed as particles and/or as molecules in said excipient matrix.

In the invention herein, an “excipient matrix” may generally be understood as the component in a composite material (e.g., a drug-containing solid) that holds a filler (e.g., a functional filler such as dispersed drug particles and/or drug molecules) together. Thus, drug particles and/or drug molecules may generally be embedded in an excipient matrix.

In some embodiments, an excipient matrix may be erodible. In the invention herein, an excipient matrix may generally be understood “erodible” if said excipient matrix erodes or dissolves upon exposure to a relevant physiological fluid (e.g., gastric fluid) under physiological conditions.

To ensure that an excipient matrix erodes or dissolves upon immersing in a relevant physiological fluid under physiological conditions, in some embodiments an excipient matrix may be formed by one or more third excipients that are soluble in a relevant physiological fluid under physiological conditions (e.g., gastric fluid, simulated gastric fluid, and so on.).

In the invention herein, a third excipient may generally be considered “soluble” if a solid particle of said third excipient dissolves upon exposure to a relevant physiological fluid under physiological conditions (e.g., gastric fluid). In preferred embodiments, moreover, the drug release rate by a drug-containing solid formed by a soluble third excipient (e.g., one or more soluble third excipients) and active ingredient particles or molecules dispersed in said soluble third excipient (e.g. said one or more soluble third excipients) is limited (e.g., substantially limited, substantially determined, etc.) by the rate at which said third excipient erodes. In some embodiments, therefore, a soluble third excipient used herein may comprise a solubility in a relevant physiological fluid (e.g., gastric fluid) under physiological conditions greater than the solubility in said physiological fluid of an active ingredient used herein. This includes, but is not limited to each of the one or more soluble third excipients forming an excipient matrix comprising a solubility in gastric fluid greater or substantially greater than a solubility in gastric fluid of an active ingredient dispersed in said excipient matrix.

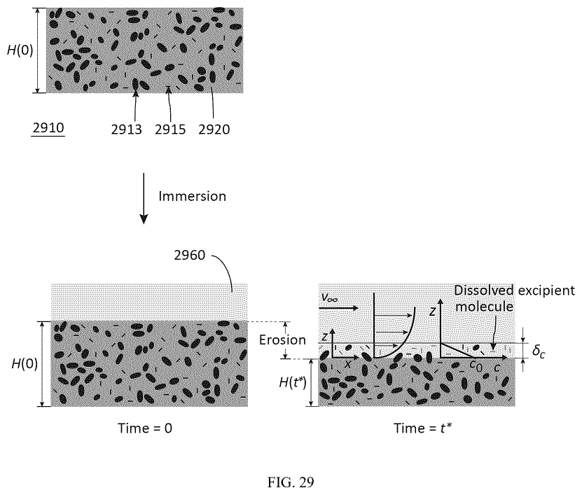

FIG. 29 presents a non-limiting example of a drug-containing solid 2910 comprising drug particles 2913 and/or drug molecules 2915 dispersed in an excipient matrix 2920. Said excipient matrix 2920 is formed by one or more third excipients that are soluble in a relevant physiological fluid 2960 (e.g., gastric fluid) under physiological conditions. Upon immersing said drug-containing solid 2910 (or a pharmaceutical solid dosage form comprising said drug-containing solid 2910) in said physiological fluid 2960 (e.g., gastric fluid) under said physiological conditions, said drug-containing solid 2910 erodes by dissolution of said erodible excipient matrix 2920, thereby releasing said drug particles 2913 and/or drug molecules 2915 into said physiological fluid 2960 over time.

More specifically, because one or more third excipients 2920 in the drug-containing solid 2910 comprise at least a third excipient 2920 that is soluble in a physiological fluid 2960 under physiological conditions (e.g., a third excipient that dissolves in said physiological fluid, or a third excipient that diffuses from drug-containing solid into said physiological fluid), said soluble third excipient 2920 may diffuse into (e.g., dissolve or erode into) said physiological fluid 2960 upon contact of said drug-containing solid 2910 with said physiological fluid 2960. As a result, due to third excipient 2920 dissolution or erosion, drug molecules 2915 (and/or drug particles 2913) that are embedded in said dissolving third excipient 2920 may be released (e.g., set free, loosened up and transferred, disattached from the drug containing solid and transferred, eroded around to lose attachment from the drug-containing solid and transferred, etc.) into the dissolution fluid 2960. In such embodiments, the drug release rate may be substantially determined by the rate at which the third excipient 2920 erodes.

Such embodiments where the drug release rate is substantially determined by the rate at which an excipient matrix erodes may be preferred herein because the drug release rate by a drug-containing solid may be controlled by the properties of said excipient matrix.

In some embodiments, therefore, one or more soluble third excipients may be the predominant third excipients an excipient matrix. In the invention herein, “one or more predominant third excipients” may generally be understood as the one or more third excipients in a drug-containing solid that determine the major properties of said drug-containing solid. By way of example but not by way of limitation, one or more soluble third excipients may be the predominant third excipients an excipient matrix of a drug-containing solid if the drug release rate by said drug-containing solid is substantially determined by the rate at which said excipient matrix erodes.

In some embodiments, furthermore, one or more soluble third excipients may be substantially connected through an excipient matrix (e.g., through an erodible excipient matrix).

In some embodiments, moreover, an erodible excipient matrix may be substantially connected through a drug-containing solid.

In some embodiments, furthermore, an erodible excipient matrix may substantially surround dispersed active ingredient particles or molecules.

In some embodiments, moreover, one or more soluble third excipients may substantially surround active ingredient particles or molecules.

Similarly, in preferable embodiments, active ingredient particles or molecules may be substantially embedded in an excipient matrix that erodes upon exposure to a physiological fluid (e.g., gastric fluid) under physiological conditions.

In some embodiments, moreover, active ingredient particles or molecules may be substantially embedded in one or more soluble third excipients.

In some embodiments, moreover, weight or volume fraction of one or more soluble third excipients in an excipient matrix (e.g., an erodible excipient matrix) may be greater than 0.3. This includes, but is not limited to weight or volume fraction of one or more soluble third excipients in an excipient matrix (e.g., an erodible excipient matrix) greater than 0.35, or greater than 0.4, or greater than 0.45, or greater than 0.5, or greater than 0.55, or greater than 0.6.

In some embodiments, moreover, at least one third excipient within a drug-containing solid comprises a solubility greater than 0.1 g/l in a relevant physiological/body fluid (e.g., gastric fluid) under physiological conditions. More preferably, at least one third excipient of a drug-containing solid material or a drug-containing phase or part of a drug-containing solid may have a solubility in a relevant physiological/body fluid (e.g., gastric fluid) under physiological conditions greater than 0.2 g/l, or more preferably greater than 0.5 g/l, or more preferably greater than 1 g/l, or more preferably greater than 2 g/l. This includes, but is not limited to at least one third excipient having a solubility in a relevant physiological/body fluid (e.g., gastric fluid) under physiological conditions greater than 5 g/l, or greater than 10 g/l, or greater than 20 g/l, or greater than 50 g/l. The solubility of a material is referred to herein as the maximum amount or mass of said material that can be dissolved at equilibrium in a given volume of physiological fluid under physiological conditions divided by the volume of said fluid or of the solution formed. By way of example but not by way of limitation, the solubility of a solute in a solvent may be determined by optical methods.

In some embodiments, moreover, at least one soluble third excipient in drug-containing solid comprises a polymer (e.g., a polymeric excipient, a polymeric material, etc.).

A non-limiting example of a soluble third excipient includes hydroxypropyl methylcellulose.

Additional non-limiting examples of soluble third excipients include excipients selected from the group comprising hydroxypropyl methylcellulose, hydroxyethyl cellulose, polyvinyl alcohol, polyvinylpyrrolidone, sodium alginate, hydroxypropyl cellulose, hydroxyethyl cellulose, methyl cellulose, hydroxypropyl methyl ether cellulose, starch, polymethacrylates (e.g., poly(methacrylic acid, ethyl acrylate) 1:1, or butylmethacrylat-(2-dimethylaminoethyl)methacrylat-methylmathacrylat-copolymer), polyethylene oxide, or vinylpyrrolidone-vinyl acetate copolymer.

In some embodiments, furthermore, at least one third excipient in drug-containing solid can be absorptive of a physiological fluid under physiological conditions. By way of example but not by way of limitation, in such embodiments drug may be released from a drug-containing solid by diffusion through an excipient matrix formed by one or more absorptive third excipients.

Moreover, in some embodiments at least one third excipient in drug-containing solid can be both absorptive of a physiological fluid under physiological conditions and soluble in said physiological fluid under said physiological conditions. Additionally, at least one third excipient in drug-containing solid may be mutually soluble with a relevant physiological fluid under physiological conditions.

A drug-containing solid comprising an excipient matrix that only consists of one or more soluble third excipients may be preferable in some embodiments herein, but not in all. By way of example but not by way of limitation, in some embodiments a drug-containing solid comprising an erodible excipient matrix that only comprises one or more soluble third excipients (e.g., one or more polymeric soluble third excipients, etc.) may erode and release drug too fast after immersing said drug-containing solid in a physiological fluid under physiological conditions.

In some embodiments, therefore, a drug-containing solid may comprise one or more stabilizing third excipients.

Generally, the properties of stabilizing third excipient in drug-containing solid herein may be similar to the properties of mechanically strengthening second excipient in mechanically strengthening semi-permeable layer herein. By way of example but not by way of limitation, the solubility of stabilizing third excipient may be limited or low in a relevant physiological fluid (e.g., gastric fluid) under physiological conditions. More specifically, in some embodiments a stabilizing third excipient has a solubility no greater than 0.5 g/l in a relevant physiological/body fluid under physiological conditions. This includes, but is not limited to at least one stabilizing third excipient having a solubility in a relevant physiological/body fluid under physiological conditions no greater than 0.4 g/l, or no greater than 0.2 g/l, or no greater than 0.1 g/l, or no greater than 0.05 g/l, or no greater than 0.02 g/l, or no greater than 0.01 g/l, or no greater than 0.005 g/l, or no greater than 0.002 g/i, or no greater than 0.001 g/l. In the extreme case, a stabilizing third excipient may be insoluble or at least practically insoluble in a relevant physiological fluid under physiological conditions. A smaller solubility of stabilizing third excipient in physiological fluid can be preferable for stabilizing or mechanically supporting a drug-containing solid immersed in a physiological fluid under physiological conditions.

In some embodiments, furthermore, a stabilizing third excipient (e.g., a film of stabilizing third excipient) comprises a tensile strength greater than 0.02 MPa after soaking with a physiological fluid (e.g., gastric fluid, etc.) under physiological conditions. This includes, but is not limited to a stabilizing third excipient comprising a tensile strength greater than 0.05 MPa, or greater than 0.08 MPa, or greater than 0.1 MPa, or greater than 0.2 MPa, or greater than 0.3 MPa, or greater than 0.4 MPa, or greater than 0.5 MPa, or greater than 0.6 MPa after soaking with a physiological fluid under physiological conditions.

In some embodiments, moreover, a stabilizing third excipient (e.g., a film of stabilizing third excipient) comprises a strain at fracture greater than 0.5 after soaking with a physiological fluid (e.g., gastric fluid, etc.) under physiological conditions. This includes, but is not limited to a stabilizing third excipient comprising a strain at fracture greater than 0.75, or greater than 1, or greater than 1.25, or greater than 1.5 after soaking with a physiological fluid under physiological conditions.

It may be noted, however, that a stabilizing third excipient may soften or plasticize somewhat upon contact with or immersion in a physiological fluid under physiological conditions. As a result, a stabilizing third excipient can be a solid in the dry state, but upon immersion in or exposure to a relevant physiological fluid (e.g., gastric fluid, etc.) under physiological conditions, it may transition to a semi-solid or viscoelastic material. Because the stiffness, yield strength, tensile strength, elongational viscosity, etc. of stabilizing third excipient should not be too large to avoid injury of the gastrointestinal mucosa, such stabilizing excipients that soften somewhat upon immersing in a physiological fluid may be preferable in some embodiments of the invention herein.

In some embodiments, at least one stabilizing third excipient comprises a polymer (e.g., a polymeric excipient, a polymeric material, etc.).

In some embodiments, a stabilizing third excipient may be selected from the group comprising methacrylic acid-ethyl acrylate copolymer.

In some embodiments, moreover, a stabilizing third excipient may be selected from the group comprising polyvinyl acetate.

In some embodiments, furthermore, a stabilizing third excipient is selected from the group comprising methacrylic acid-ethyl acrylate copolymer, methacrylic acid-methyl methacrylate copolymer, ethyl acrylate-methylmethacrylate copolymer, hydroxypropyl methylcellulose acetate succinate, polyvinyl acetate, polymers including methacrylic acid, polymers including ethyl acrylate, polymers including methyl methacrylate, polymers including methacrylate, Poly[Ethyl acrylate, methyl methacrylate, trimethylammonioethyl methacrylate chloride], and ethylcellulose.

To control the rate at which drug is released, moreover, in some embodiments drug-containing solid comprises a mixture of one or more soluble third excipients and one or more stabilizing third excipients.

In some embodiments, moreover, one or more soluble third excipients and one or more stabilizing third excipients may form a solid solution. Thus, in the invention herein an excipient matrix (e.g., an erodible excipient matrix) may comprise a solid solution of one or more soluble third excipients and one or more stabilizing third excipients.

In some embodiments, moreover, an excipient matrix may comprise a single-phase material. In the invention herein, a single-phase material may generally be understood as a material with uniform or substantially uniform properties and/or with uniform or substantially uniform structure throughout its volume.

Without wishing to be bound to a particular theory, it may be noted that generally, the greater a volume or weight fraction of stabilizing third excipient in a mixture of soluble and stabilizing third excipient in a drug-containing solid is, the slower may the erosion rate or the drug release rate of said drug containing solid be upon immersing it in a physiological fluid under physiological conditions. Thus, to achieve desirable drug release rates herein, the volume or weight fraction of stabilizing third excipient in a drug-containing solid (or in a mixture of soluble and stabilizing third excipient) may be limited.

In some embodiments, therefore, the volume fraction and/or the weight fraction of one or more stabilizing third excipients in a mixture of one or more soluble third excipients and one or more stabilizing third excipients in a drug-containing solid may be no greater than 0.7 (e.g., no greater than 0.65, or no greater than 0.6, or no greater than 0.55, or no greater than 0.5, or no greater than 0.45, or no greater than 0.4, or no greater than 0.35).

In some embodiments, moreover, the volume fraction and/or the weight fraction of one or more soluble third excipients in a mixture of one or more soluble third excipients and one or more stabilizing third excipients in a drug-containing solid may greater than 0.3 (e.g., greater than 0.35, or greater than 0.4, or greater than 0.45, or greater than 0.5, or greater than 0.55, or greater than 0.6, or greater than 0.65).

However, to assure that stabilizing third excipient adequately stabilizes a drug-containing solid after immersing in a physiological fluid, the volume fraction and/or the weight fraction of stabilizing third excipient in said drug-containing solid may not be too small. In some embodiments, therefore, the volume fraction and/or the weight fraction of one or more stabilizing third excipients (e.g., one or more stabilizing third excipients in their totality) in a mixture of one or more soluble third excipients and one or more stabilizing third excipients in a drug-containing solid may be no less than 0.002 (e.g., no less than 0.005, or no less than 0.007, or no less than 0.01, or no less than 0.02). It may be noted that the limitations of volume and weight fractions given above may refer to the volume or weight of a mixture of one or more soluble third excipients and one or more stabilizing third excipients in a drug-containing solid. They may not refer to the volume or weight of the entire drug-containing solid.

In some embodiments, moreover, upon immersing drug-containing solid comprising a mixture of one or more soluble third excipients and one or more stabilizing third excipients in a physiological fluid under physiological conditions, said one or more stabilizing third excipients may form a solid or semi-solid network mechanically supporting the drug-containing solid and said one or more soluble polymeric third excipients may transition to a viscous mass or a viscous solution or a solution that dissolves over time.

In some embodiments, drug is dispersed as particles and/or molecules in a mixture of one or more stabilizing third excipients and one or more soluble third excipients.

### (k) Structure of Expandable, Drug Releasable Solid

Generally, in the invention herein an expandable, drug releasable solid may comprise at least a fluid-absorptive solid core (e.g, at least a three dimensional structural framework of one or more fluid-absorptive elements, etc.), at least a mechanically strengthening semi-permeable layer, and at least a drug containing solid. Said mechanically strengthening semi-permeable layer may substantially encapsulate said solid core. Also, at least part of said mechanically strengthening semi-permeable layer may be disposed between said solid core and said drug-containing solid. In some embodiments, moreover, an expandable, drug releasable solid may further comprise one or more free spaces or channels within its exterior volume.

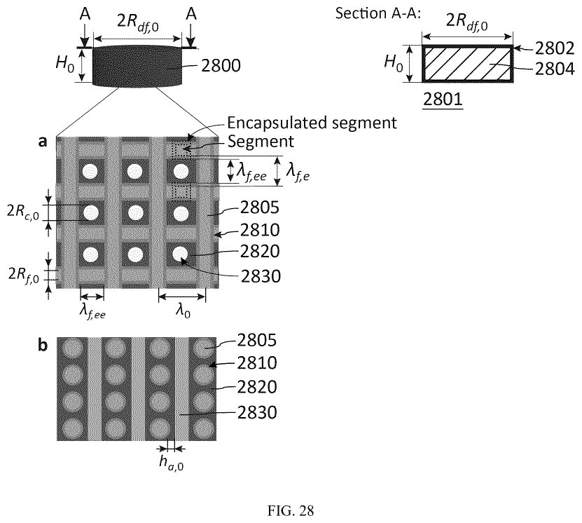

FIG. 28 presents a non-limiting example of an expandable, drug releasable solid 2801 of a pharmaceutical solid dosage form 2800 according to this invention. The expandable, drug releasable solid 2801 may comprise an outer surface 2802 and an internal structure 2804 contiguous with and terminating at said outer surface 2802. Said internal structure 2804 may comprise at least an expandable, three dimensional structural framework of one or more fluid-absorptive structural elements 2805, a mechanically strengthening semi-permeable layer 2810, a drug-containing solid 2820, and one or more free spaces or channels 2830. Said structural framework 2805 may be substantially encapsulated by said mechanically strengthening layer 2810. Said encapsulated structural framework of one or more elements 2805,2810 may comprise encapsulated segments spaced apart from adjoining encapsulated segments by encapsulated-element-free spacings, λ_{f,ee}, defining one or more encapsulated-element-free spaces within the expandable, drug releasable solid 2801. At least part of said drug-containing solid 2820 may be attached to said encapsulated structural framework 2805,2810 and occupy encapsulated-element-free space within the expandable, drug releasable solid 2801. Said one or more encapsulated-element-free spaces within the expandable, drug releasable solid 2801 may further comprise one or more free spaces or channels 2830.

In preferred embodiments, one or more free spaces or one or more channels may be substantially open to an exterior surface of an expandable, drug releasable solid. In some embodiments, moreover, one or more free spaces or one or more channels may have at least one open end contiguous with and terminating at an exterior or outer surface of an expandable, drug releasable solid (or of an encapsulated, three-dimensional structural framework of elements or fibers). In some embodiments, moreover, one or more channels may have at least two open ends contiguous with and terminating at an exterior or outer surface of an expandable, drug releasable solid (or of an encapsulated, three-dimensional structural framework of elements or fibers). If one or more free spaces or one or more channels are open to an exterior surface of an expandable, drug releasable solid, physiological fluid may percolate into the interior of the internal structure of said expandable, drug releasable solid upon contact with said physiological fluid.

In some embodiments, moreover, one or more free spaces or one or more channels may be substantially connected through an exterior dimension of an expandable, drug releasable solid or through an exterior dimension of an encapsulated structural framework of one or more elements.

In some embodiments of the invention herein, one or more free spaces within an exterior volume of an expandable, drug releasable solid or within an exterior volume of an encapsulated three dimensional structural framework of elements may be considered “substantially connected” if a fluid can substantially percolate said one or more free spaces upon immersing said drug releasable solid or said encapsulated framework (or parts or sections of said drug releasable solid or said encapsulated framework) in said fluid.

In some embodiments of the invention herein, moreover, one or more free spaces within an exterior volume of an expandable, drug releasable solid or within an exterior volume of an encapsulated three dimensional structural framework of elements may be considered “substantially connected” if they extend over a length at least about a third of an exterior dimension of said an expandable, drug releasable solid or of said encapsulated structural framework. This includes, but is not limited to one or more free spaces within an exterior volume of an expandable, drug releasable solid or of an encapsulated three-dimensional structural framework of elements extending over a length at least two thirds of an exterior dimension of said expandable, drug releasable solid or encapsulated structural framework, or over a length at least equal to an exterior dimension of said expandable, drug releasable solid. In preferred embodiments, a free space may extend through an exterior dimension (e.g., a thickness, etc.) of an expandable, drug releasable solid or of an encapsulated three-dimensional structural framework of fluid-absorptive elements.

Thus, in some embodiments, at least one encapsulated-element-free space may have an open channel through it. Similarly, in some embodiments, at least one encapsulated-fiber-free space may have an open channel through it.

In some embodiments, moreover, drug-containing solid may occupy encapsulated-element-free space between a free space or channel (e.g., an open free space or an open channel) and an encapsulated three dimensional structural framework elements. Thus, in some embodiments, a free space (e.g., a channel) may be substantially surrounded by a drug-containing annulus (e.g., an annulus formed by a drug-containing solid). Similarly, in some embodiments, a drug-containing annulus may substantially surround a free space or channel.

In some embodiments, furthermore, one or more channels may be substantially straight. Also, in some embodiments, one or more channels may comprise a substantially uniform cross section along their length. In some embodiments, furthermore, one or more channels may comprise a substantially uniform cross section along their length.

In some embodiments, moreover, a plurality of channels may be substantially parallel to each other. By way of example but not by way of limitation, in some embodiments, at least two channels may be substantially parallel to each other. This includes, but is not limited to a plurality of at least three channels, or at least four channels, or at least five channels, or at least six channels, etc. being substantially parallel to each other.

In some embodiments, furthermore, a plurality of channels may be substantially orderly arranged within an expandable, drug releasable solid. Similarly, in some embodiments, a plurality of channels may be arranged in a substantially ordered pattern within an expandable, drug releasable solid. By way of example but not by way of limitation, in some embodiments a plurality of channels may be arranged in a substantially square lattice (as shown, for example, in the non-limiting top views of FIGS. 5 and 7).

Any more details of the structure of an expandable, drug releasable solid would be obvious to a person of ordinary skill in the art. All of them are included in this invention.

### (l) Free Spacing in Expandable, Drug Releasable Solid or Pharmaceutical Solid Dosage Form

Typically, moreover, for physiological or dissolution fluid to percolate into the interior of the internal structure of an expandable, drug releasable solid the channel size or diameter (e.g., channel width, or pore size, or free spacing, or effective free spacing) within free space of an expandable, drug releasable solid may be on the micro- or macro-scale. Thus, in some embodiments, the free spacing, λ_{f}, (e.g., the channel size or channel diameter) through (e.g., along, across, etc.) a free space of an expandable, drug releasable solid may be greater than 1 μm. This includes, but is not limited to λ_{f} greater than 1.25 μm, or greater than 1.5 μm, or greater than 1.75 μm, or greater than 2 μm, or greater than 5 μm, or greater than 7 μm, or greater than 10 μm, or greater than 15 μm, or greater than 20 μm, or greater than 25 μm, or greater than 30 μm, or greater than 40 μm, or greater than 50 μm.

Because the dosage form volume is generally limited and for other reasons, however, the effective free spacing may not be too large. Thus, in some embodiments, the free spacing (e.g., average free spacing) through (e.g., along, across, etc.) free space of an expandable, drug releasable solid may be in the ranges 1 μm-5 mm, 1 μm-3 mm, 1.25 μm-5 mm, 1.5 μm-5 mm, 1.5 μm-3 mm, 5 μm-2.5 mm, 10 μm-2 mm, 10 μm-4 mm, 5 μm-4 mm, 10 μm-3 mm, 15 μm-3 mm, 20 μm-3 mm, 30 μm-4 mm, 40 μm-4 mm, or 50 μm-4 mm.

The free spacing may be determined experimentally from microstructural images (e.g., scanning electron micrographs, micro computed tomography scans, and so on) of the pharmaceutical solid dosage form or of an expandable, drug releasable solid. Non-limiting examples describing and illustrating how a free spacing may be determined from microstructural images are presented in the experimental examples or described and illustrated in the U.S. application Ser. No. 15/482,776 titled “Fibrous dosage form”.

It may be noted, that in some embodiments herein the free spacing within one or more free spaces of an expandable, drug releasable solid may be precisely controlled.

Any more details of free spacings in free space of an expandable, drug releasable solid would be obvious to a person of ordinary skill in the art. All of them are included in this invention.

### (m) Composition of Free Space

Generally, one or more free spaces (e.g., one or more interconnected free spaces) may be filled with a matter that is removable by a physiological fluid under physiological conditions. Such matter that is removable by a physiological fluid under physiological conditions can, for example, be a gas which escapes a free space upon percolation of said free space by said physiological fluid. Such matter that is removable by a physiological fluid under physiological conditions can, however, also be a solid that is highly soluble in said physiological fluid, and thus dissolves rapidly upon contact with or immersion in said physiological fluid. In some embodiments, such matter that is removable by a physiological fluid under physiological conditions can even comprise a liquid.

In preferred embodiments, a biocompatible gas that may fill free space includes air. Other non-limiting examples of biocompatible gases that may fill free space include nitrogen, CO₂, argon, oxygen, and nitric oxide, among others.

Non-limiting examples of solids that are removed or dissolved after contact with physiological/body fluid include sugars or polyols, such as Sucrose, Fructose, Galactose, Lactose, Maltose, Glucose, Maltodextrin, Mannitol, Maltitol, Isomalt, Lactitol, Xylitol, Sorbitol, among others. Other examples of solids include polymers, such as polyethylene glycol, polyvinyl pyrrolidone, polyvinyl alcohol, among others. Typically, a solid material should have a solubility in physiological/body fluid (e.g., an aqueous physiological or body fluid) under physiological conditions greater than about 5 g/l to be removed or dissolved rapidly after contact with dissolution medium. This includes, but is not limited to a solubility greater than 10 g/l, or greater than 15 g/l, or greater than 20 g/l, or greater than 50 g/l. The diffusivity of the solid material (as dissolved molecule in physiological/body fluid under physiological conditions) should typically be greater than about 2×10⁻¹² m²/s if the solid material must be dissolved rapidly after contact with dissolution medium. This includes, but is not limited to a diffusivity in physiological fluid under physiological conditions greater than 4×10⁻¹² m²/s, or greater than 6×10⁻¹² m²/s, or greater than 8×10⁻¹¹ m²/s, or greater than 1×10⁻¹¹ m²/s, or greater than 2×10⁻¹¹ m²/s.

In some embodiments, moreover, a solid that may fill free space has a molecular weight (e.g., average molecular weight, such as number average molecular weight or weight average molecular weight) no greater than about 80 kg/mol. This includes, but is not limited to a molecular weight (e.g., average molecular weight, such as number average molecular weight or weight average molecular weight) no greater than 70 kg/mol, or no greater than 60 kg/mol, or no greater than 50 kg/mol, or no greater than 45 kg/mol, or no greater than 40 kg/mol, or no greater than 35 kg/mol.

Further compositions of free space obvious to a person of ordinary skill in the art who is given all information of this specification are all included in this invention.

### (n) Expansion Properties of Dosage Form or Expandable Drug Releasable Solid

Generally, upon immersing a pharmaceutical solid dosage form, expandable, drug releasable solid, encapsulated solid core, solid core, etc. as disclosed herein in a physiological fluid under physiological conditions, said a pharmaceutical solid dosage form, expandable, drug releasable solid, encapsulated solid core, solid core, etc. may expand. By way of example but not by way of limitation, expansion of said a pharmaceutical solid dosage form, expandable, drug releasable solid, encapsulated solid core, solid core, etc. may prevent its premature passage through the pylorus after ingestion. This may enable said a pharmaceutical solid dosage form, expandable, drug releasable solid, encapsulated solid core, solid core, etc. to release drug into the stomach over time.

Generally, moreover, a pharmaceutical solid dosage form, expandable, drug releasable solid, encapsulated solid core, solid core, etc. as disclosed herein may expand with fluid absorption upon immersing said a pharmaceutical solid dosage form, expandable, drug releasable solid, encapsulated solid core, solid core, etc. in a physiological fluid under physiological conditions. In preferred embodiments, moreover, upon exposure of the a pharmaceutical solid dosage form, expandable, drug releasable solid, encapsulated solid core, solid core, etc. to a physiological fluid, the pharmaceutical solid dosage form, expandable, drug releasable solid, encapsulated solid core, solid core, etc. may expand primarily with fluid absorption. In the invention herein, a solid is generally understood as “expanding primarily with fluid absorption” if upon exposure of said solid to a physiological fluid, the greatest expansion of said solid (e.g., the greatest longitudinal expansion, such as the greatest increase in length or normalized length; the greatest volumetric expansion, such as the greatest increase in volume or normalized volume; etc.) is mostly or primarily due to the absorption of said physiological fluid. It may be noted that the surface layer-encapsulated solid core may generally transition to a viscous (e.g., a highly viscous) or semi-solid mass as it expands with fluid absorption.

In some embodiments, moreover, upon exposure of a dosage form or an expandable, drug releasable solid herein to a physiological fluid under physiological conditions, a mechanically strengthening, semi-permeable layer may expand due to an internal pressure in the core it may substantially encapsulate. In some embodiments, moreover, said internal pressure may be generated by osmotic flow of fluid into said core.

In some embodiments, an expanded expandable, drug releasable solid may form a substantially continuous or connected structure with an exterior dimension of the order of or greater than the diameter of the pylorus (or of the pyloric sphincter) of the human subject (or of the animal) by which the dosage form was ingested.

More specifically, in some embodiments, at least one exterior dimension of an expanded expandable, drug releasable solid may be greater than 16 mm. This includes, but is not limited to such preferred embodiments where at least one exterior dimension of an expanded solid or semi-solid may be greater than 17 mm, or greater than 18 mm, or greater than 19 mm, or even greater than 20 mm.

In some embodiments of the invention herein, moreover, at least one exterior dimension (e.g., a length, width, thickness, side length, etc.) of a dosage form or expanded, expandable, drug releasable solid herein expands to at least 1.2 times the initial value (e.g., the initial length prior to exposure to said physiological fluid) after immersing in a physiological or body fluid under physiological conditions. This includes, but is not limited to at least one exterior dimension of a dosage form or expanded expandable, drug releasable solid expanding to at least 1.25 times the initial value, or at least 1.3 times the initial value, or at least 1.35 times the initial value, or at least 1.4 times the initial value, or at least 1.45 times the initial value, or at least 1.5 times the initial value after immersing in or exposing to a physiological or body fluid under physiological conditions.

In some embodiments of the invention herein, moreover, at least one dimension (e.g., a side length or the thickness) of a pharmaceutical solid dosage form or expandable, drug releasable solid (e.g., an encapsulated solid core, a solid core, etc.) may expand to at least 1.2 times the initial value (e.g., the initial length prior to exposure to said physiological fluid) within no more than 500 minutes of immersing in a physiological or body fluid under physiological conditions. This includes, but is not limited to at least one dimension of a pharmaceutical solid dosage form or expandable, drug releasable solid (e.g., an encapsulated solid core, a solid core, etc.) reaching a length at least 1.2 times the initial length within no more than 300 minutes, or within no more than 200 minutes, or within no more than 150 minutes, or within no more than 100 minutes, or within no more than 50 minutes, or within no more than 40 minutes, or within no more than 30 minutes of immersing in said physiological or body fluid under physiological conditions. This may also include, but not be limited to at least one dimension of a pharmaceutical solid dosage form or expandable, drug releasable solid (e.g., an encapsulated solid core, a solid core, etc.) expanding to a length at least 1.3 times the initial length, or at least 1.4 times the initial length, or at least 1.45 times the initial length, or at least 1.5 times the initial length, or at least 1.55 times the initial length, or at least 1.6 times the initial length within no more than 300 minutes of immersing in or exposing to a physiological or body fluid under physiological conditions.

Furthermore, in some embodiments a pharmaceutical solid dosage form or expandable, drug releasable solid (e.g., an encapsulated solid core, a solid core, etc.) may expand to at least 2 times its initial volume within no more than about 500 minutes of immersing in a physiological or body fluid under physiological conditions. This includes, but is not limited to a pharmaceutical solid dosage form or expandable, drug releasable solid (e.g., an expandable solid core, a solid core, etc.) expanding to at least 2 times, or at least 3 times, or at least 4 times, or at least 4.5 times, or at least 5 times, or at least 6 times, or at least 6.5 times its initial volume within no more than about 300 minutes of immersing in a physiological or body fluid under physiological conditions.

In some embodiments, upon exposure of the dosage form to a physiological fluid, an expandable, drug releasable solid may form an expanded, viscoelastic composite mass.

In some embodiments, moreover, upon prolonged exposure to a physiological fluid (e.g., longer than 2, 4, 6, 8, or 10 hours in a lightly stirred dissolution fluid such as acidic water), an expanded expandable, drug releasable solid, or an expanded framework or semi-solid or viscoelastic mass may maintain its length between 1.3 and 4 times the initial length (e.g., the initial length prior to exposure to said physiological fluid) for prolonged time.

In some embodiments, an expanded expandable, drug releasable solid or a viscoelastic or semi-solid mass may comprise a substantially continuous or connected network of one or more strength-enhancing excipients.

More embodiments related to the expansion of the disclosed dosage forms or expandable solids obvious to a person of ordinary skill in the art are all within the spirit and scope of this invention.

### (o) Mechanical Properties of Expanded Expandable Drug Releasable Solid

For maintaining an expanded solid or semi-solid in the stomach of a human or animal subject for prolonged time, it may have adequate mechanical properties.

In some embodiments, therefore, an expanded solid or semi-solid (e.g., an expanded expandable, drug releasable solid) comprises a tensile strength (or a fracture strength) greater than 0.006 MPa for maintaining said expanded solid or semi-solid in the stomach of a human or animal subject for prolonged time. This includes, but is not limited to an expanded solid or semi-solid having a tensile strength (or a fracture strength) greater than 0.008 MPa, or greater than 0.01 MPa, or greater than 0.02 MPa, or greater than 0.03 MPa, or greater than or greater than 0.04 MPa, or greater than 0.05 MPa.

In some embodiments, moreover, an expanded solid or semi-solid (e.g., a viscoelastic composite mass, an expanded expandable, drug releasable solid or dosage form, etc.) formed after immersing an expandable, drug releasable solid in a physiological fluid under physiological conditions comprises an elastic modulus greater than 0.0005 MPa. This includes, but is not limited to an expanded solid or semi-solid formed after immersing an expandable, drug releasable solid in a physiological or body fluid under physiological conditions comprising an elastic modulus greater than 0.002 MPa, or greater than 0.003 MPa, or greater than 0.005 MPa, or greater than 0.007 MPa, or greater than 0.01 MPa, or greater than 0.02 MPa, or greater than 0.03 MPa, or greater than 0.04 MPa, or greater than 0.045 MPa, or greater than 0.05 MPa, or greater than 0.055 MPa, or greater than 0.06 MPa, or greater than 0.065 MPa, or greater than 0.07 MPa, or greater than 0.075 MPa.

The elastic modulus of an expanded solid or semi-solid (e.g., a viscoelastic composite mass, an expanded expandable, drug releasable solid or dosage form, etc.) may, however, also be limited to prevent injury of the gastrointestinal mucosa. In some embodiments, therefore, an expanded solid or semi-solid (e.g., a viscoelastic composite mass, an expanded expandable, drug releasable solid or dosage form, etc.) formed after immersing an expandable, drug releasable solid in a physiological fluid under physiological conditions comprises an elastic modulus no greater than 100 MPa. This includes, but is not limited to an expanded solid or semi-solid (e.g., a viscoelastic composite mass, an expanded expandable, drug releasable solid or dosage form, etc.) formed after immersing an expandable, drug releasable solid in a physiological fluid under physiological conditions comprising an elastic modulus no greater than 80 MPa, or no greater than 70 MPa, or no greater than 60 MPa, or no greater than 50 MPa, or no greater than 40 MPa, or no greater than 30 MPa, or no greater than 20 MPa, or no greater than 10 MPa.

In some embodiments, therefore, an expanded solid or semi-solid (e.g., a viscoelastic composite mass, an expanded expandable, drug releasable solid or dosage form, etc.) formed after immersing an expandable, drug releasable solid in a physiological fluid under physiological conditions comprises a highly elastic or viscoelastic mass that temporarily may not break or permanently deform in a stomach (e.g., under the compressive forces of stomach walls, etc.). Eventually, however, the expanded solid or semi-solid may disintegrate or break, and be excreted from the gastrointestinal tract and from the body.

More embodiments related to the mechanical properties of disclosed expanded solids or semi-solids obvious to a person of ordinary skill in the art are all within the spirit and scope of this invention.

### (p) Drug Release Properties of Expandable Drug Releasable Solid

Generally, the disclosed dosage form may be particularly useful for releasing drug into a physiological fluid over prolonged time. In some embodiments, therefore, upon immersing a pharmaceutical solid dosage form or expandable, drug releasable solid as disclosed herein in a physiological fluid under physiological conditions, eighty percent of the content of an active ingredient or drug in said expandable, drug releasable solid may be released into said physiological fluid within a time no less than about 1.5 hours of immersing said expandable, drug releasable solid in said physiological fluid under physiological conditions. This includes, but is not limited to eighty percent of the content of an active ingredient or drug in an expandable, drug releasable solid released into a physiological fluid within a time no less than 2 hours, or no less than 3 hours, or no less than 5 hours, or no less than 7 hours, or greater than 8 hours, or greater than 10 hours, or greater than 12 hours, or greater than 14 hours of immersing said expandable, drug releasable solid in said physiological fluid under physiological conditions.

The drug release time may, however, also not be too long. In some embodiments, therefore, upon immersing a pharmaceutical solid dosage form or expandable, drug releasable solid as disclosed herein in a physiological fluid under physiological conditions, eighty percent of the content of an active ingredient or drug in said expandable, drug releasable solid may be released into said physiological fluid within a time no greater than about 150 hours of immersing said expandable, drug releasable solid in said physiological fluid under physiological conditions. This includes, but is not limited to eighty percent of the content of an active ingredient in an expandable, drug releasable solid released into a physiological fluid within a time no greater than 120 hours, or no greater than 100 hours, or no greater than 90 hours, or no greater than 80 hours, or no greater than 70 hours, or no greater than 60 hours, or no greater than 50 hours, or no greater than 40 hours, or no greater than 35 hours, or no greater than 30 hours of immersing said expandable, drug releasable solid in said physiological fluid under physiological conditions.

Also, in some embodiments, upon immersing a pharmaceutical solid dosage form or drug releasable solid as disclosed herein in a relevant physiological fluid under physiological conditions, an amount or mass of an active ingredient released from said pharmaceutical solid dosage form or drug releasable solid into said physiological fluid may increase substantially linearly with or substantially in proportion to the time of immersing said pharmaceutical solid dosage form or drug releasable solid in said physiological fluid under said physiological conditions.

Similarly, In some embodiments an amount or mass of a drug released from said pharmaceutical solid dosage form into said physiological fluid increases substantially linearly with or substantially in proportion to the time of immersing said pharmaceutical solid dosage form in said physiological fluid under said physiological conditions.

More embodiments related to the drug release properties of disclosed dosage forms or expandable, drug releasable solids obvious to a person of ordinary skill in the art are all within the spirit and scope of this invention.

### (q) Use of the Disclosed Dosage Form

Generally, the disclosed dosage form may be used for delivering drug to a human or animal body to treat a myriad of diseases or medical conditions. Generally, moreover, the dosage form can be used for delivering any (or almost any) drug or active ingredient. The disclosed dosage form can, however, be particularly useful for delivery of some specific drugs.

By way of example but not by way of limitation, the disclosed dosage form can be particularly useful for controlled or prolonged delivery of drugs that are sparingly or very sparingly soluble in intestinal fluid.

In some embodiments, therefore the pharmaceutical solid dosage form disclosed herein comprises at least an active ingredient or drug having a solubility in an aqueous physiological fluid at a pH in the range 5.5 to 7.5 (e.g., at a pH in the range 6-7.5) under physiological conditions (e.g., intestinal fluid) no greater than 0.5 mg/ml. This includes, but is not limited to at least an active ingredient or drug having a solubility in an aqueous physiological fluid at a pH in the range 5.5 to 7.5 (e.g., at a pH in the range 6-7.5) under physiological conditions (e.g., intestinal fluid) no greater than 0.3 mg/ml, or no greater than 0.2 mg/ml, or no greater than 0.1 mg/ml, or no greater than 0.075 mg/ml, or no greater than 0.05 mg/ml, or no greater than 0.03 mg/ml, or no greater than 0.02 mg/ml, or no greater than 0.01 mg/ml, or no greater than 0.0075 mg/ml, or no greater than 0.005 mg/ml.

Similarly, the disclosed dosage form can be particularly useful for controlled or prolonged delivery of drugs that comprise a pH-dependent solubility.

In some embodiments, therefore, at least an active pharmaceutical ingredient or drug comprises a solubility in an aqueous physiological fluid at pH in the range 1 to 2 under physiological conditions (e.g., gastric fluid) greater than a solubility in an aqueous physiological fluid at pH in the range 5.5 to 7.5 (e.g., at a pH in the range 6-7.5) under physiological conditions (e.g., intestinal fluid). This includes, but is not limited to a solubility of a drug in an aqueous physiological fluid at pH in the range 1 to 2 under physiological conditions (e.g., gastric fluid) greater than 2 times (or greater than 3 times, or greater than 4 times, or greater than 5 times, or greater than 6 times, or greater than 8 times, or greater than 10 times, or greater than 15 times, or greater than 20 times, or greater than 30 times, or greater than 50 times, or greater than 100 times) a solubility in an aqueous physiological fluid at pH in the range 5.5 to 7.5 (e.g., at a pH in the range 6-7.5) under physiological conditions (e.g., intestinal fluid).

Moreover, the invention herein can be highly useful for prolonged delivery of drugs with short elimination half-life or elimination time constant. In some embodiments, therefore, at least one active pharmaceutical ingredient or drug comprises an elimination half life or elimination time constant no greater than 50 hours. This includes, but is not limited to at least one active ingredient or drug comprising an elimination half life or elimination time constant no greater than 45 hours, or no greater than 40 hours, or no greater than 35 hours, or no greater than 30 hours, or no greater than 25 hours, or no greater than 20 hours, or no greater than 15 hours, or no greater than 14 hours, or no greater than 13 hours, or no greater than 12 hours, or no greater than 11 hours, or no greater than 10 hours.

The invention herein can be further highly useful for delivering drugs that inhibit a kinase or a mutated kinase (e.g., a tyrosine kinase or a mutated tyrosine kinase, etc.) in a human or animal body. In some embodiments, therefore, the active pharmaceutical ingredient herein comprises an inhibitor of a kinase and/or a mutated kinase (e.g., a tyrosine kinase and/or a mutated tyrosine kinase, etc.). In the invention herein, the term “kinase” includes all kinases including tyrosine kinases, janus kinases, mutated tyrosine kinases, mutated janus kinases, and so on. The term “tyrosine kinase” includes all tyrosine kinases, including mutated tyrosine kinases, and so on. A non-limiting example of a tyrosine kinase inhibitor is nilotinib (including all salts (e.g., nilotinib hydrochloride monohydrate, etc.), crystalline forms, etc. thereof) and any other combinations or forms thereof. In some embodiments, therefore, at least one active ingredient in a drug-containing solid herein comprises nilotinib.

The disclosed dosage form may further be particularly useful for treating cancer or a neoplastic disease. By way of example but not by way of limitation, a neoplastic disease or cancer may be treated with an active ingredient from the group comprising kinase inhibitors, kinase inhibiting drugs, janus kinase inhibitors, janus kinase inhibiting drugs, tyrosine kinase inhibitors, tyrosine kinase inhibiting drugs and so on.

The dosage forms herein may also be useful for achieving or maintaining a substantially constant drug concentration in the blood or at a biological target site of a human or animal subject. In some embodiments, therefore, upon immersing a pharmaceutical solid dosage form or a drug releasable solid as disclosed herein in a physiological fluid under physiological conditions, an amount or mass of an active ingredient released from said pharmaceutical solid dosage form or drug releasable solid into said physiological fluid may increase substantially linearly with or substantially in proportion to the time of immersing said pharmaceutical solid dosage form or drug releasable solid in said physiological fluid under said physiological conditions.

It may be obvious to a person of ordinary skill in the art that the dosage form disclosed herein may comprise additional embodiments, features, materials, excipients, active ingredients, and so on. Any such embodiments, features, materials, excipients, active ingredients, and so on obvious to a person of ordinary skill in the art are all within the spirit and scope of this invention.

### EXPERIMENTAL EXAMPLES

The following examples present non-limiting ways by which disclosed dosage forms may be prepared and analyzed, and may enable one of skill in the art to more readily understand the principle of the invention. The examples also include ways for preparing and analyzing particulate dosage forms. The examples are presented by way of illustration and are not meant to be limiting in any way.

### Example 1: Preparation of Particulate Dosage Forms

### (a) Materials for Preparing Particulate Dosage Forms

The materials used for preparing the particulate dosage forms were as follows.

Drug powder formulation: A particulate mixture of nilotinib hydrochloride monohydrate and various excipients, extracted from marketed immediate-release nilotinib capsules (trade name: Tasigna). The immediate-release nilotinib capsules were purchased from Novartis, Basel, Switzerland, through the pharmacy of the Vetsuisse Faculty at the University of Zurich.

Contrast agent: Barium sulfate (BaSO₄), purchased as solid particles of size ˜1 μm, from Humco, Austin, TX.

Empty capsules: Gelatin capsules of size 00 (trade name: Interdelta), purchased from Capsugel, La Seyne sur Mer, France.

### (b) Preparation of Particulate Dosage Forms

First, the contents of a marketed immediate-release capsule containing a particulate mixture of 200 mg nilotinib and 200 mg of various excipients were removed. Then 170 mg of the contrast agent (BaSO₄ particles) were added and mixed with the contents. The mixture was filled into an empty capsule that was subsequently closed. Thus, as listed in Table 3, the final particulate dosage form contained 200 mg nilotinib, 200 mg excipients, and 170 mg BaSO₄.

### Example 2: Preparation of Gastroretentive Dosage Forms

### (a) Materials for Preparing Gastroretentive Dosage Forms

The materials for preparing the gastroretentive dosage forms were as follows.

Excipients in the fluid-absorptive core (e.g., fluid-absorptive fibers): Hydroxypropyl methylcellulose with a number-average molecular weight of about 120 kg/mol (HPMC2), purchased from Merck KGaA, Darmstadt, Germany; methacrylic acid-ethyl acrylate copolymer (1:1), with a molecular weight of about 250 kg/mol (trade name: Eudragit L100-55), received from Evonik, Essen, Germany.

Contrast agent in the core: Barium sulfate (BaSO₄), purchased as solid particles of size ˜1 μm, from Humco, Austin, TX.

Strengthening coating: Methacrylic acid-ethyl acrylate copolymer as in the core.

Excipients in the drug-containing solid: Hydroxypropyl methylcellulose with a molecular weight of 10 kg/mol (HPMC1), purchased from Merck KGaA, Darmstadt, Germany; and methacrylic acid-ethyl acrylate copolymer as in the core.

Drug: Nilotinib hydrochloride monohydrate, purchased as solid particles from the European Directorate for the Quality of Medicine (EDQM), Strasbourg, France.

Solvents: Dimethylsulfoxide (DMSO) and acetone.

### (b) Preparation of Fluid-Absorptive Core (e.g., Fibrous Cylindrical Disks)

First, solid particles of HPMC2, Eudragit L100-55, and barium sulfate were mixed with liquid DMSO to form a uniform suspension. The concentrations of HPMC2, Eudragit L100-55, and barium sulfate were 500, 300, and 343 mg/ml of DMSO.

The suspension was extruded through a laboratory extruder to form a uniform viscous paste. The viscous paste was put in a syringe equipped with a hypodermic needle of inner radius, Rₙ=205 μm. The paste was extruded through the needle to form a wet fiber that was patterned layer-by-layer in a cross-ply structure. The nominal inter-fiber spacing was 1500 μm and the number of layers was 32.

After patterning, the solvent was evaporated by blowing warm air at about 50° C. and a velocity of about 1 m/s over the structure for a day. Finally, a cylindrical disk with nominal diameter 14 mm and thickness 8 mm was punched out.

The solid fibrous cylindrical disk consisted 43.75 wt % HPMC2, 26.25 wt % Eudragit L100-55, and 30 wt % barium sulfate, Table 3 later.

It may be noted that the solid fibrous cylindrical disks may generally be understood herein as a “solid core”, an “expandable solid core”, a “fluid-absorptive core”, an “expandable framework”, and so on.

### (c) Coating the Fluid-Absorptive Core (e.g., the Fibers of the Fibrous Cylindrical Disks)

The cylindrical disks so produced were dip-coated with a fiber-strengthening, enteric coating solution. The coating solution consisted of Eudragit L100-55 and acetone at a polymer concentration of 100 mg/ml. The coating was applied by dipping the disks into the coating solution for about 5-10 seconds. The coated disks were then put in a vacuum chamber held at about 35° C. To evaporate the solvent the pressure was slowly reduced from atmospheric to 200 Pa, and maintained at this value for about two hours. After solvent evaporation, the inter-fiber spaces were opened by pushing a 0.8 mm diameter needle through them. The dipping-evaporation process was executed three times.

It may be noted that a coated cylindrical disk may generally be understood as an “expandable solid” or an “expandable, gastroretentive solid” herein.

(d) Attaching Drug-Containing Solid to the Coated Framework (e.g., Filling the Spaces Between Coated Fibers with Drug)

First, solid particles of nilotinib, HPMC1, and Eudragit L100-55 were mixed with liquid DMSO to form a uniform dispersion. The weight fractions of nilotinib, HPMC1, Eudragit L100-55, and DMSO in the dispersion were 0.372, 0.223, 0.025, and 0.38, respectively.

The coated cylindrical disk was then introduced in a cylindrical mold with a diameter of 14 mm. Subsequently, about 540 mg of the dispersion was dispensed on the disk and pressed into the inter-fiber spaces with a piston. After filling, circular channels were formed in the inter-fiber spaces by pushing a 0.72 mm diameter needle through them. To evaporate the solvent and solidify the dispersion the sample was put in a vacuum chamber maintained at a pressure of 200 Pa and a temperature of 20° C. for about a day.

After solvent evaporation, the composition of the inter-fiber space was 60 wt % nilotinib, 36 wt % HPMC1, and 4 wt % Eudragit L100-55, Table 3.

It may be noted, moreover, that after solvent evaporation drug-containing solid in the inter-fiber spaces was attached to the coated cylindrical disk.

After solvent evaporation, preparation of the non-limiting experimental gastroretentive dosage forms herein was final. It may be noted, however, that the dosage forms could have been post-processed further. Any such post-processing obvious to a person of ordinary skill in the art is included in this invention.

### Example 3: Microstructure of Particulate Dosage Forms

The microstructure of drug-excipient particles of the particulate dosage forms was imaged by a Zeiss Merlin High Resolution SEM with a GEMINI column. Prior to imaging the particles were dispensed on carbon conductive tape and coated with a 10-nm thick layer of gold. The sample was imaged with an in-lens secondary electron detector. The accelerating voltage was 5 kV, and the probe current 95 pA.

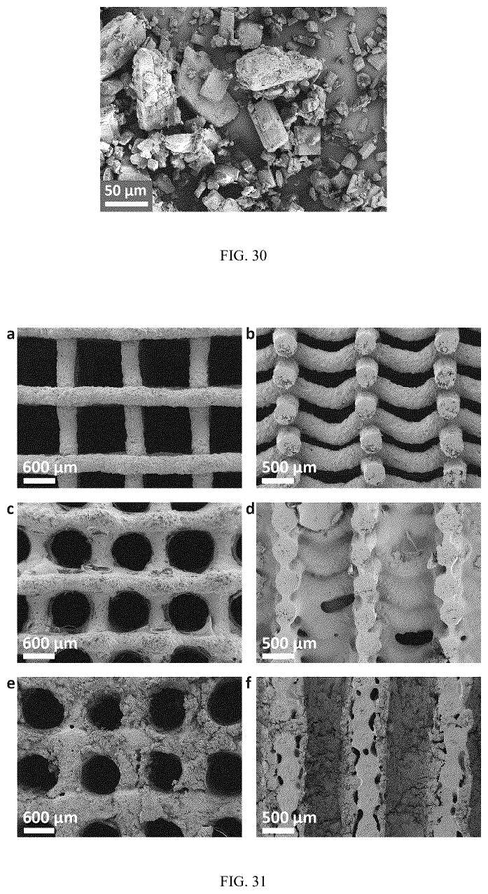

FIG. 30 is a scanning electron micrograph of the mixture of drug and excipient particles of the particulate dosage form. The volume-based average particle radius was about 18.5 μm, Table 3.

### Example 4: Microstructures and Weights of Fibrous Disks and Gastroretentive Dosage Forms

The microstructures of uncoated fibrous cylindrical disks (e.g., uncoated fluid-absorptive cores), coated fibrous cylindrical disks (e.g., coated or encapsulated cores), and “final” dosage forms were imaged by a Zeiss Merlin High Resolution SEM with a GEMINI column. The top surfaces of the fibrous structures and gastroretentive dosage forms were imaged after coating the sample with a 10-nm thick layer of gold. The longitudinal sections of the disks and dosage forms were imaged after the sample was cut with a thin blade (MX35 Ultra, Thermo Scientific, Waltham, MA) and coated with gold as above. All specimens were imaged with an in-lens secondary electron detector. The accelerating voltage was 5 kV, and the probe current 95 pA.

The weights of the uncoated fibrous cylindrical disks, the coated fibrous cylindrical disks, and the final dosage forms were measured by an analytical balance with a resolution of 0.1 mg (Mettler Toledo, Greifensee, Switzerland).

FIGS. 31a and 31b show the top and longitudinal sectional views of the microstructure of an uncoated fibrous cylindrical disk. The fiber radius, R_{f,0}=165 μm, and the inter-fiber spacing, λ₀=1298 μm, Table 1. The weight of the uncoated fibrous cylindrical disk was 500 mg, Table 3.

The microstructure of a coated fibrous cylindrical disk is shown in FIGS. 31c and 31d. The coating surrounded the fibers, and bridged the neighboring fibers vertically, but not horizontally. Thus, the microstructure of the coated fibrous cylindrical disk may be approximated as having vertical walls of thickness, 2R_{f,0}, and vertical square channels of width, λ₀−2R_{f,0}. The weight of the coated fibrous cylindrical disk was 650 mg, Table 3.

TABLE 3
Selected microstructural parameters and
composition of the particulate and fibrous dosage forms.

| | | |
|---|---|---|
| Symbol | Description | Value |

Particulate

| | | | |
|---|---|---|---|
| M_{BaSO4} | mass of barium sulfate in dosage form | 170 | mg |
| M_{d,0} | mass of drug in dosage form | 200 | mg |
| Mₑ | mass of excipients (other than barium sulfate) in dosage form | 200 | mg |
| R_{p,0} | volume-based average particle radius | 18.5 | μm ^{a} |

Fibrous

| H₀ | thickness of solid dosage form | 8 | mm |
|---|---|---|---|
| h_{a,0} | thickness of drug-laden annulus | 156 | μm ^{a} |
| M_{d,0} | mass of drug in dosage form | 200 | mg ^{b} |
| M_{df} | mass of final dosage form | 984 | mg ^{c} |
| M_{ec} | mass of enteric coated fibrous cylindrical disk | 650 | mg ^{c} |
| M_{f} | mass of uncoated fibrous cylindrical disk | 500 | mg ^{c} |
| nₐ or n_{c} | number of annuli or channels in dosage form | 91.4 ^{d} | |
| nₗ | number of fiber layers in dosage form | 32 | |
| R_{c,0} | channel radius | 302 ± 32 μm ^{a} | |
| R_{df,0} | radius of dosage form | 7 | mm |

| | | |
|---|---|---|
| | fiber radius | 165 ± 6 μm ^{a} |
| w_{d,a} | weight fraction of drug in annulus | |
| w_{HPMC1,a} | weight fraction of low-molecular-weight HPMC | 0.36 |
| | (HPMC1) in annulus | |
| w_{ee,a} | weight fraction of enteric excipient (Eudragit L100-55) in annulus | 0.04 |
| w_{HPMC2,f} | weight fraction of high-molecular-weight HPMC (HPMC2) in fiber | 0.6 |
| | core | |
| w_{ee,f} | weight fraction of enteric excipient in fiber core | 0.36 |
| w_{BaSO4,f} | weight fraction of BaSO₄ in fiber core | 0.04 |
| λ₀ | inter-fiber distance | 1298 ± 54 μm ^{a} |
| φ_{ec} | volume fraction of enteric coating in dosage form | 0.15 ^{e} |
| φ_{f} | volume fraction of solid fiber core in dosage form | 0.3 ^{f} |

^{a} From the scanning electron micrographs shown in FIGS. 30 and 31.
^{b} From Eq. (33).
^{c} From weight measurements.
^{d} Calculated as nₐ = n_{c} = πR_{df,0}²/λ₀².
^{e} From Eq. (32) using a density of the enteric coating, ρ_{ec} = 800 kg/m³.
^{f} From Eq. (31) using a density of the solid fibers, ρ_{f} = 1367 kg/m³.

FIGS. 31e and 31f are the images of the “final” dosage forms with coated fibers and drug-filled inter-fiber space. At the center of the inter-fiber space the dosage forms had an open, cylindrical channel with radius, R_{c,0}=302 sm. Between the channel and the coated fibers was a drug-loaded annulus with wall thickness, h_{a,0}≈156 μm. The weight of the final dosage form was 984 mg, Table 3.

Several additional parameters may be obtained as follows. The volume fraction of fibers in the dosage form may be written as:

$${φ}_{f} = \frac{{V}_{f}}{{V}_{df}} = \frac{{M}_{f}}{{ρ}_{f}} \times \frac{1}{π {R}_{df , 0}^{2} {H}_{0}} \qquad ( 31 )$$

where V_{f} is the volume of fibers in the dosage form, V_{df} the volume of the dosage form, M_{f} the mass of the uncoated fibrous cylindrical disk, ρ_{f} the density of the solid fibers, R_{df,0} the radius of the solid fibrous dosage form, and H₀ its thickness. Substituting the relevant parameters listed in Table 3 in Eq. (31), φ_{f}=0.3.

Similarly, the volume fraction of enteric coating in the dosage form may be written as:

$${φ}_{ec} = \frac{{V}_{ec}}{{V}_{df}} = \frac{{M}_{ec} - {M}_{f}}{{ρ}_{ec}} \times \frac{1}{π {R}_{df , 0}^{2} {H}_{0}} \qquad ( 32 )$$

where V_{ec} is the volume of enteric coating in the dosage form, M_{ec} the mass of the coated fibrous cylindrical disk, and ρ_{ec} the density of the solid coating. For the relevant parameters listed in Table 3, by Eq. (32) φ_{ec}=0.15.

The drug mass in the dosage form may be obtained as:

$${M}_{d , 0} = {w}_{d , a} ( {M}_{df} - {M}_{ec} ) \qquad ( 33 )$$

where w_{d,a} is the weight fraction of drug in the annuli and Mg the mass of the final dosage form. For the relevant parameters (w_{d,a}=0.6, M_{df}=984 mg, and M_{ec}=650 mg), by Eq. (33) M_{d,0}=200 mg, Table 3.

### Example 5: In Vito Disintegration of Particulate Dosage Forms

The particulate dosage form was immersed in a beaker filled with 400 ml dissolution fluid (0.03 M hydrochloric acid (HCl) in deionized (DI) water, pH=1.5, at 37° C.). The fluid was stirred with a paddle rotating at 70 rpm. The immersed sample was then imaged periodically by a Nikon DX camera.

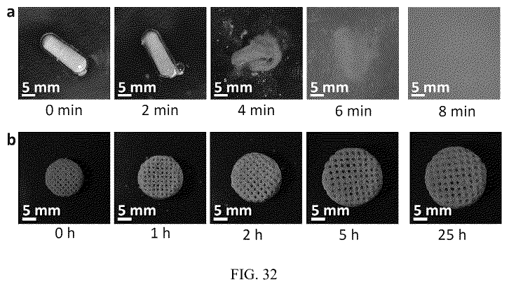

FIG. 32a presents images of the particle-filled capsule after immersion in a stirred dissolution fluid. The capsule broke apart after 2-4 minutes, and released drug and excipient particles. By 6-8 minutes the particles were uniformly dispersed in the dissolution fluid.

### Example 6: In Vitro Expansion of Gastroretentive Dosage Forms

For an analysis of in vitro expansion, the gastroretentive dosage form was immersed in a beaker filled with 400 ml dissolution fluid (0.03 M HCl in DI water, pH=1.5, at 37° C.). The fluid was stirred with a paddle rotating at 70 rpm. The immersed sample was then imaged periodically by a Nikon DX camera.

FIG. 32b presents experimental top-view images of a fibrous dosage form after immersing in a dissolution fluid. The dosage form expanded in less than 5 hours and formed a viscoelastic composite structure that was stable for more than a day.

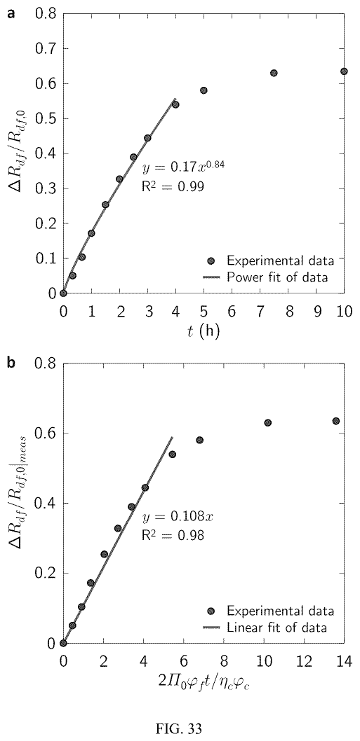

FIG. 33a plots the measured normalized radial expansion of the dosage form, ΔR_{df}/R_{df,0}, versus time. As predicted by Eq. (3), in the time interval [0, 4]h, the data increased roughly linearly with time as ΔR_{df}/R_{df,0}=0.17t^{0.84}, Table 4. After the linear increase, ΔR_{df}/R_{df,0} plateaued out to 0.63 by 7.5 hours.

FIG. 33b plots the measured normalized radial expansion of the dosage form (from FIG. 33a) versus 2Π₀φ_{f}t/φ_{ec}η (from Eq. (3)) using the non-limiting parameter values Π₀=12.84 kPa, φ_{f}=0.3, φ_{ec}=0.15, and η=1.36×10⁸ Pa·s. In the time interval [0, 4]h, ΔR_{df}/R_{df,0|meas}=0.108×2π₀φ_{f}t/φ_{ec}η. Thus, Eq. (3) reasonably describes the normalized radial expansion of the fibrous dosage forms if the constant, k₂=0.108.

### Example 7: Mechanical Strength of Expanded Gastroretentive Dosage Forms

For determining the mechanical strength of the expanded dosage forms, the dosage forms were first soaked in a dissolution fluid (0.03 M HCl in DI water, pH=1.5, at 37° C.) for 10 hours. Diametral compression tests were then conducted using a Zwick Roell mechanical testing machine equipped with a 10 kN load cell and compression platens. The relative velocity of the platens was 2 mm/s. The test was stopped when the specimen fractured visibly.

FIG. 34 is a series of images of diametral compression of an expanded fibrous dosage form after soaking in the dissolution fluid for 10 hours. Upon compression, the dosage form deformed, and as the load was released it eventually regained a shape and size comparable to that of the original expanded dosage form. However, a crack was seen along the axis of loading after compression.

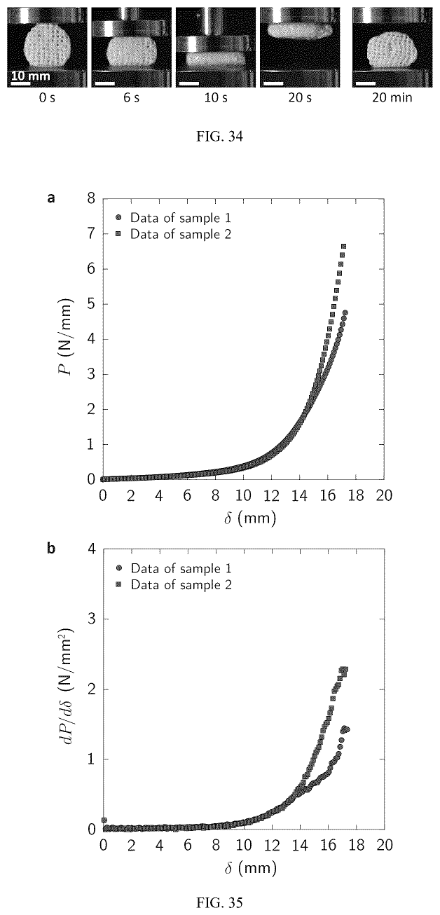

FIG. 35a presents the measured load per unit thickness, P, versus displacement, δ, during diametral compression, and FIG. 35b plots the corresponding slope, dP/dδ versus δ. Up to a displacement of about 10 mm the load intensity increased little with displacement, to 0.35 N/mm at 10 mm. But after that both P and dP/dδ increased greatly. At about 17 mm displacement the slope exhibited a notable kink, an indication of the onset of fracture (see, e.g., A. H. Blaesi, D. Kümmerlen, H. Richter, N. Saka, Mechanical strength and gastric residence time of expandable fibrous dosage forms, Int. J. Pharm. 613 (2022) 120792). The average load intensity at the onset of fracture, P_{f,df}, of the two samples was 5.58 N/mm, Table 4. This is reasonably close the calculated value by Eq. (4), 0.175 MPa, Table 4. Thus, the model may be fairly reasonable.

### Example 8: In Vitro Drug Release by the Particulate and Gastroretentive Fibrous Dosage Forms

Drug release by the dosage forms was determined using a USP dissolution apparatus II (Sotax AG, Aesch, Switzerland). The dissolution bath of the apparatus was filled with 1200 ml dissolution fluid (0.03 M HCl in DI water, pH=1.5, at 37° C.). The fluid was stirred with a paddle rotating at 50 rpm. The dosage forms were immersed in the fluid, and the concentration of dissolved drug versus time was measured by UV absorption using a fiber optic probe connected to a Cary 60 UV-vis spectrophotometer (Agilent Technologies, Santa Clara, CA). Drug concentrations were determined by subtracting the UV absorbance at the wavelength 310 nm from the absorbance at 300 nm.

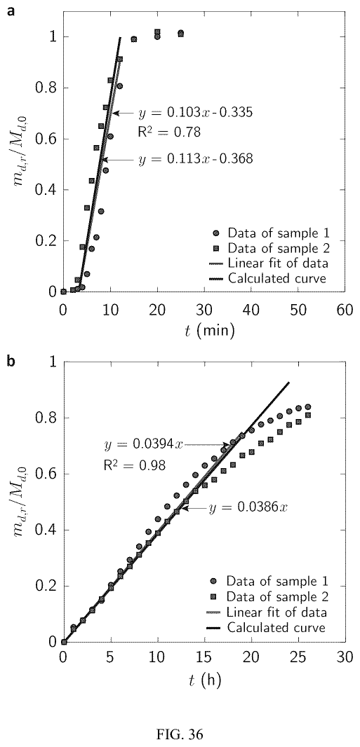

FIG. 36a plots the fraction of drug released, m_{d,r}/M_{d,0}, by the particulate dosage forms versus time. Up to the dissolution of the capsule at 3 minutes, no drug was released. But after that m_{d,r}/M_{d,0} increased rapidly and at roughly constant rate. Eighty percent of the drug was dissolved in just 11 minutes (0.18 h), Table 4. The linear fit equation in the time interval [0.05,0.22]h ([3,13] min) was m_{d,r}/M_{d,0}=6.18t−0.335, Table 4. After the linear increase m_{d,r}/M_{d,0} plateaued out to 1.

By contrast, the gastroretentive fibrous dosage forms released drug much slower than the particulate forms, FIG. 36b. Seventy percent of the drug was released in about 18.8 hours. Up to this time m_{d,r}/M_{d,0} increased roughly linearly with time as m_{d,r}/M_{d,0}=0.0394t, two orders of magnitude slower than upon immersion of the particulate forms. After the linear increase m_{d,r}/M_{d,0} plateaued out to 1. Eighty percent of the drug was released in 24 hours, Table 4.

### Example 9: Determination of the Gastric Residence Time of the Dosage Forms in Dogs

Six experiments comprising four particulate and two fibrous dosage forms were conducted. The experiments were done with four healthy beagle dogs (12.6-16.1 kg). All four dogs received a particulate dosage form each. Additionally, two of them received a fibrous dosage form each.

The dogs fasted for 18 hours prior to the experiment. The dosage form was administered to an awake dog with 20 ml water. After administration, the dogs were periodically placed in an x-ray permeable box to monitor the position of the dosage form in the gastrointestinal tract by biplanar fluoroscopy (using a Philips Allura Clarity fluoroscopy system). Between imaging, the dogs were allowed to roam about freely with access to water.

At 3 hours after administering the dosage form, 180 grams of basic dry food was given (Grainfree 25/17, Petzeba AG, Alberswil, Switzerland). No sedatives, anesthesia, or other supplements were administered immediately before, during, or after the experiment.

The study was conducted in compliance with the Swiss Animal Welfare Act (TSchG, 2005) and the Swiss Animal Welfare Ordinance (TSchV, 2008). It was approved by the Swiss Federal Veterinary Office Zurich; the animal license number was ZH072/2021.

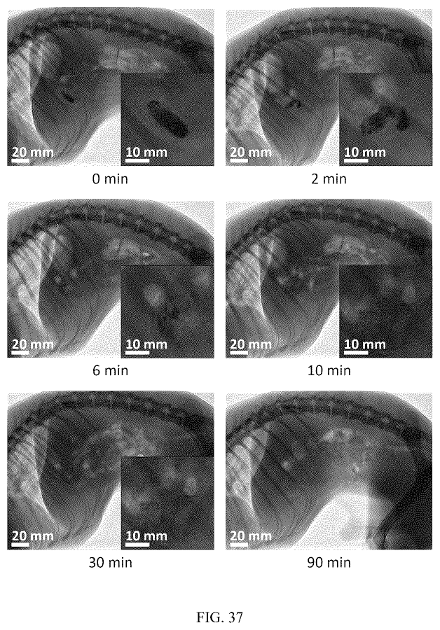

FIG. 37 presents fluoroscopic images of the position, shape, and size of the particle-filled capsule after administering to a dog. The capsule reached the stomach unimpaired. But already after 2 minutes in the stomach, it was fragmented and released contrast agent into the gastric fluid. By 6 minutes, the capsule was essentially disintegrated. As predicted by the model Eqs. (10) and (11), residual contrast agent was seen in the stomach up to 60-90 minutes after ingestion, Table 5. By 90 minutes (1.5 h) the stomach was mostly empty.

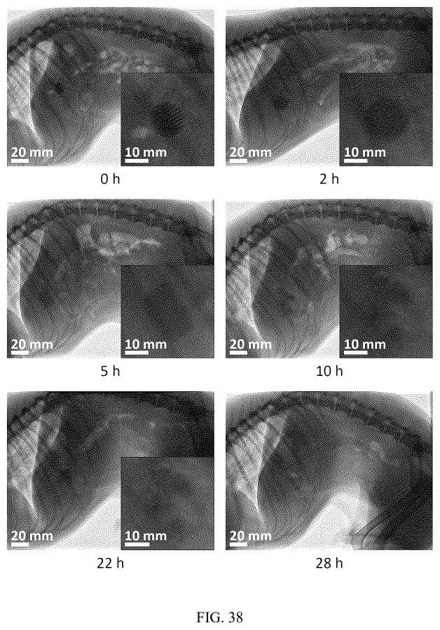

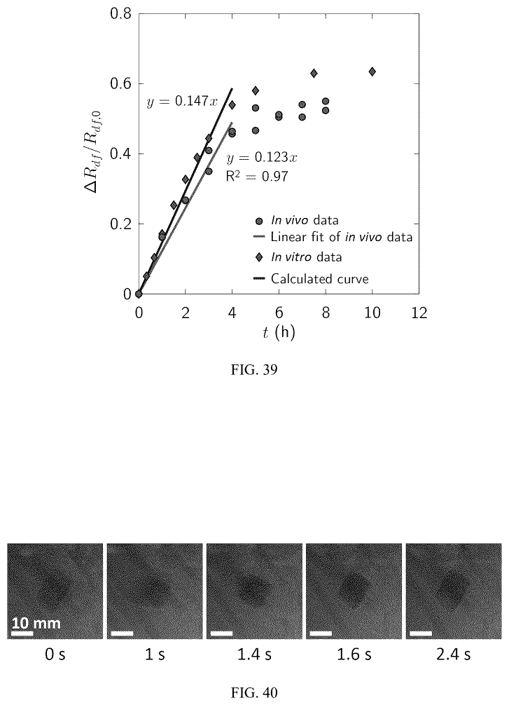

FIG. 38 shows fluoroscopic images of the position, shape, and size of a fibrous dosage form at various times after administering to a dog. The measured in vivo normalized radial expansion (from the fluoroscopic images) and the in vitro data (from Example 6, FIG. 33) are shown in FIG. 39. Up to 3-4 hours of expansion the in vivo and in vitro data increased linearly with time at comparable rates. Thereafter the data plateaued out; the in vivo data to about 0.54 and the in vitro data to 0.63.

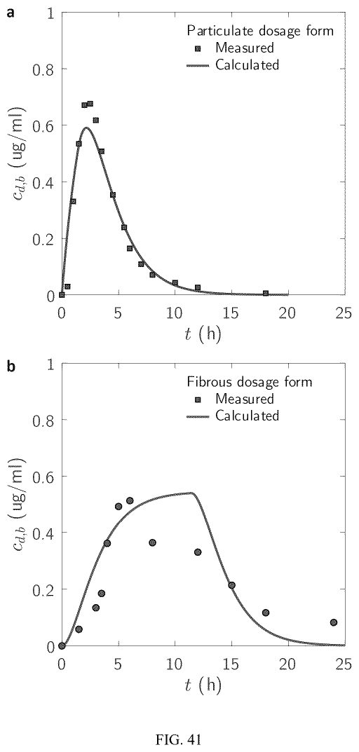

FIG. 40 is a fluoroscopic image sequence illustrating the compression-decompression cycle of an expanded fibrous dosage form in the stomach of a dog 6 hours after administration. At the beginning of the sequence, the diameter of the dosage form was about 21 mm. After 1.4 s, however, the dosage form was diametrically compressed by about 6 mm to a diameter of 15 mm. Subsequently, the dosage form sprang back, and roughly regained its original width at 2.4 s.

The compression-decompression cycles were not observed in the first few hours after administering the dosage form. But after 3-4 hours, when the dosage form had expanded and transitioned to a viscoelastic composite mass, they were observed about every 10 seconds.

Not surprisingly, therefore, as shown in FIG. 38 and listed in Table 5, in the experiments the fibrous dosage form broke apart after about 10 hours. Thereafter the fragments gradually passed into the intestine. The amount of fragments in the stomach decreased substantially within the first few hours after fracture. Small amounts of fragments could, however, be seen in the stomach until about 23.5 hours after administering the dosage form, Table 5.

TABLE 4
Calculated and measured quantities after immersing particulate and
fibrous dosage forms in a dissolution fluid.

| | Particulate dosage form | | Fibrous dosage form | |
|---|---|---|---|---|
| Quantity | Calculated | Measured | Calculated | Measured |
| ΔR_{df}/R_{df,0} (−) | — | — | C₂t | 0.17t^{0.84} |
| tₑ (h) | — | — | — | 4 |
| ΔR_{df}/R_{df,0}\|_{t = te} (−) | — | — | — | 0.54 |
| P_{f,df} (N/mm) | — | — | — | 5.58 |
| σ_{f,df} (MPa) | — | — | 0.175 | 0.169 |
| m_{d,r}/M_{d,0} | 6.79t | 6.18t | 0.0386t | 0.0394 |
| dm_{d,r}/dt (mg/h) | 1.36 × 10³ | 1.24 × 10³ | 7.72 | 7.88 |
| t_{0.8} (h) | 0.17 | 0.18 | 20.7 | 24.1 |

ΔR_{df} R_{df,0}: normalized radial expansion of fibrous dosage form;
tₑ: time to reach ″terminal″ expansion;
ΔR_{df}/R_{df,0}|ₜ₌ₜₑ: ″terminal″ normalized expansion;
P_{f,df}. load intensity at fracture;
σ_{f,df}. tensile stress at fracture;
m_{d,r}/M_{d,0}: fraction of drug released;
dm_{d,r}/dt: drug release rate
t_{0.8}: time to dissolve 80 percent of the drug;
The unit of time, t, is hours.
The rate constant, C₂, obtained by subsituting the non-limiting parameters Π₀ = 12.84 kPa, φ_{f} = 0.3, φ_{ec} = 0.15, and η = 1.36 × 10⁸ Pa·s in Eq. (3) is k₂ × 1.36/h. From the linear fit of FIG. 33b, k₂ = 0.108. Thus, C₂ = 0.147/h.
The measured load intensities at fracture of the individual fibrous dosage forms were 4.76 and 6.4 N/mm, FIG. 35.
The measured t_{0.8} times of the individual particulate dosage forms were 10 and 12 minutes (0.17 and 0.2 h), FIG. 36a.
The measured t_{0.8} times of the individual fibrous dosage forms were 22.7 and 25.6 h, FIG. 36b.

TABLE 5
Calculated and measured quantities after
administering particulate and fibrous dosage forms to dogs.

| | Particulate dosage form | | Fibrous dosage form | |
|---|---|---|---|---|
| Quantity | Calculated | Measured | Calculated | Measured |
| t_{r,p} (h) | 1.43 | 1-1.5 | — | — |
| tₑ (h) | — | — | — | 4 |
| ΔR_{df}/R_{df,0}\|_{t = te} (−) | — | — | 0.59 | 0.46 |
| t_{f} (h) | — | — | — | 10^{*} |
| t_{r,f} (h) | — | — | 11.43 | 23.5^{**} |
| tₘₐₓ (h) | 2.2 | 2.5 | 11.43 | 6 |
| cₘₐₓ (μg/ml) | 0.59 | 0.68 | 0.54 | 0.51 |
| w_{1/2} (h) | 4.3 | 3.62 | 11.5 | 10.20 |

t_{r,p}: gastric residence time of drug particles;
tₑ: time to reach ″terminal″ expansion;
ΔR/R₀|_{t = te}: ″terminal″ normalized expansion;
t_{f}. time to fracture the fibrous dosage form in vivo;
t_{rf.} gastric residence time of fibrous dosage form;
tₘₐₓ: time at which drug concentration in blood is maximal;
cₘₐₓ: maximum concentration of drug in blood;
w_{1/2}: width of the peak at half height.
Calculated values of trp, ΔR/R₀|_{t = te}, and t_{r,f} are from Eqs. (11), (3), and (16).
Measured values of t_{r,p}, tₑ, ΔR/R₀|_{t = te}, and t_{r,f} are from fluoroscopic images as shown in FIGS. 37 and 38.
Calculated and measured values of tₘₐₓ, cₘₐₓ, and w_{1/2} are from FIGS. 23 and 24.
* The measured fracture times of the individual samples were 9.5 and 10 hours, respectively.
** The measured t_{r,f} was considered the time until residual fragments were seen in the stomach. The t_{r,f} times of the individual samples were 22 and 25 hours, respectively.
The data represent averages of four particulate and two fibrous dosage forms.

### Example 10: Determination of the Concentration of Drug in the Blood Plasma of Dogs

Blood samples were collected using a central venous catheter that was surgically inserted into the dog at least 48 hours before administering the dosage form. After administering, blood samples were taken at various times and blood plasma was extracted as detailed in companion work (see, e.g., A. H. Blaesi, H. Richter, and N. Saka, Gastroretentive fibrous dosage forms for prolonged delivery of sparingly soluble tyrosine kinase inhibitors. Part 4: Experimental validation of the models of drug concentration in blood, to be published in the International Journal of Pharmaceutics, and referred to herein as “REF. [4]”). The nilotinib concentration in the plasma was then measured using liquid chromatography coupled to tandem mass spectrometry (LC-MS/MS) as described in companion work (see, e.g., REF. [4]).

FIG. 41a plots the measured drug concentration in blood plasma after administering the particulate dosage forms. After a delay of about 20-30 minutes, the drug concentration increased at roughly constant rate to a peak value, cₘₐₓ=0.68 μg/ml by 2.5 hours, Table 5. The drug concentration then decreased exponentially to 0.16 μg/ml by 6 hours, and essentially zero by 12 hours. The width of the peak at half-height, w_{1/2}=3.62 h. The measured data points roughly followed the calculated curve.

FIG. 41b plots the measured drug concentration in blood plasma after administering the fibrous dosage forms. After administering the fibrous forms, the concentration increased slower; it reached a maximum of 0.51 μg/ml by 6 hours, Table 5. After 12 hours the concentration started decreasing exponentially, to 0.117 μg/ml by 18 hours and 0.083 μg/ml by 24 hours. The width of the peak at half-height was 10.2 h, about thrice of that of the particulate forms. Again, the measured data points roughly followed the calculated curve.

Thus, the fibrous dosage forms enable prolonged drug delivery into the blood at a controlled and/or constant rate, even for drugs that are substantially insoluble in intestinal fluids.

The below claims are suggestive and are not meant to be limiting the spirit and scope of the invention in any way.

We claim:

1. A pharmaceutical solid dosage form comprising:
an expandable, three dimensional structural framework of one or more substantially orderly arranged fluid-absorptive structural elements, a mechanically strengthening semi-permeable layer, and a drug-containing solid;
said structural framework of one or more elements comprising segments spaced apart from adjoining segments by element-free spacings defining one or more element-free spaces within an outer volume of said structural framework;
said structural framework further substantially encapsulated by said mechanically strengthening semi-permeable layer; and
at least part of said mechanically strengthening semi-permeable layer disposed between one or more elements or segments of said structural framework and said drug-containing solid.

2. The dosage form of claim 1, wherein one or more element-free spaces within a three dimensional structural framework of one or more fluid-absorptive elements are substantially connected through an exterior dimension of said structural framework, and wherein the average element-free spacing through one or more element-free spaces is in the range 10 μm-5 mm.

3. The dosage form of claim 1, wherein a substantially encapsulated structural framework of one or more fluid-absorptive elements comprises substantially encapsulated segments spaced apart from adjoining substantially encapsulated segments by encapsulated-element-free spacings defining one or more encapsulated-element-free spaces within an outer volume of said substantially encapsulated framework.

4. The dosage form of claim 3, wherein drug-containing solid occupies encapsulated-element-free space.

5. The dosage form of claim 4, wherein one or more encapsulated-element-free spaces within an outer volume of a substantially encapsulated framework of one or more elements further comprise one or more free spaces or one or more channels.

6. The dosage form of claim 5, wherein at least one free space is substantially connected within or through an exterior dimension of said substantially encapsulated structural framework of one or more fluid-absorptive elements.

7. The dosage form of claim 5, wherein the average free spacing through one or more free spaces or channels is in the range 5 μm-3 mm.

8. The dosage form of claim 5, wherein drug-containing solid occupies encapsulated-element-free space between a free space or channel and an encapsulated element or segment of said substantially encapsulated three-dimensional structural framework.

9. The dosage form of claim 1, wherein drug-containing solid is bonded or attached to a mechanically strengthening semi-permeable layer forming at least part of said substantially encapsulated structural framework of one or more elements.

10. The dosage form of claim 1, wherein the composition of a fluid-absorptive element includes at least a fluid-absorptive first excipient in which the solubility of gastric fluid is greater than 400 mg/ml under physiological conditions.

11. The dosage form of claim 1, wherein the composition of a fluid-absorptive element includes hydroxypropyl methylcellulose.

12. The dosage form of claim 1, wherein the composition of a fluid-absorptive element includes at least a fluid-absorptive first excipient, said fluid-absorptive first excipient selected from the group consisting of hydroxypropyl methylcellulose, hydroxyethyl cellulose, polyvinyl alcohol, polyvinylpyrrolidone, sodium alginate, hydroxypropyl cellulose, hydroxyethyl cellulose, methyl cellulose, hydroxypropyl methyl ether cellulose, starch, polymethacrylates (e.g., poly(methacrylic acid, ethyl acrylate) 1:1, or butylmethacrylat-(2-dimethylaminoethyl)methacrylat-methylmathacrylat-copolymer), polyethylene oxide, xanthan gum, or vinylpyrrolidone-vinyl acetate copolymer.

13. The dosage form of claim 1, wherein a mechanically strengthening semi-permeable layer forms a viscoelastic, semi-permeable membrane upon exposure to or soaking with gastric fluid under physiological conditions, said viscoelastic, semi-permeable membrane being substantially permeable to gastric fluid under physiological conditions.

14. The dosage form of claim 1, wherein a mechanically strengthening semi-permeable layer forms a viscoelastic, semi-permeable membrane upon exposure to or soaking with gastric fluid under physiological conditions, said viscoelastic, semi-permeable membrane being substantially impermeable to at least a fluid-absorptive first excipient included in one or more gastric fluid-exposed or gastric fluid-soaked structural elements.

15. The dosage form of claim 1, wherein a mechanically strengthening semi-permeable layer comprises a tensile strength greater than 0.05 MPa after soaking with gastric fluid under physiological conditions.

16. The dosage form of claim 1, wherein the composition of a mechanically strengthening semi-permeable layer comprises at least a strengthening second excipient, said strengthening second excipient comprising a solubility no greater than 1 g/l in gastric fluid under physiological conditions.

17. The dosage form of claim 1, wherein the composition of a mechanically strengthening semi-permeable layer comprises at least a strengthening second excipient, and solubility of gastric fluid in said strengthening second excipient further being no greater than 700 mg/ml under physiological conditions.

18. The dosage form of claim 1, wherein the composition of a mechanically strengthening semi-permeable layer comprises methacrylic acid-ethyl acrylate copolymer.

19. The dosage form of claim 1, wherein the composition of a mechanically strengthening semi-permeable layer comprises at least a strengthening second excipient, said strengthening second excipient selected from the group consisting of methacrylic acid-ethyl acrylate copolymer, methacrylic acid-methyl methacrylate copolymer, ethyl acrylate-methylmethacrylate copolymer, hydroxypropyl methylcellulose acetate succinate, polyvinyl acetate, polymers including methacrylic acid, polymers including ethyl acrylate, polymers including methyl methacrylate, polymers including methacrylate, Poly[Ethyl acrylate, methyl methacrylate, trimethylammonioethyl methacrylate chloride], and ethylcellulose.

20. The dosage form of claim 1, wherein a drug containing solid comprises at least an active pharmaceutical ingredient or drug dispersed as drug particles and/or as drug molecules in one or more third excipients, said one or more third excipients comprising at least one excipient having a solubility greater than 1 mg/ml in gastric fluid under physiological conditions and forming a matrix that erodes upon exposure to said gastric fluid.

21. The dosage form of claim 20, wherein at least one third excipient that is soluble in gastric fluid under physiological conditions comprises hydroxypropyl methylcellulose.

22. The dosage form of claim 20, wherein at least one third excipient that is soluble in gastric fluid under physiological conditions is selected from the group consisting of hydroxypropyl methylcellulose, hydroxyethyl cellulose, polyvinyl alcohol, polyvinylpyrrolidone, sodium alginate, hydroxypropyl cellulose, hydroxyethyl cellulose, methyl cellulose, hydroxypropyl methyl ether cellulose, starch, polymethacrylates (e.g., poly(methacrylic acid, ethyl acrylate) 1:1, or butylmethacrylat-(2-dimethylaminoethyl)methacrylat-methylmathacrylat-copolymer), polyethylene oxide, or vinylpyrrolidone-vinyl acetate copolymer.

23. The dosage form of claim 5, wherein a free space or channel is filled with a matter selected from the group comprising gas, liquid, or solid, or combinations thereof, and wherein said matter is partially or entirely removed upon contact with gastric fluid under physiological conditions.

24. A pharmaceutical solid dosage form comprising:
a fluid-absorptive, expandable solid core comprising at least a first pharmaceutical excipient, a mechanically strengthening semi-permeable layer comprising at least a second pharmaceutical excipient, and a drug-containing solid comprising at least an active ingredient and at least a third pharmaceutical excipient;
said fluid-absorptive, expandable solid core substantially encapsulated by said mechanically strengthening semi-permeable layer; and
at least part of said mechanically strengthening semi-permeable layer disposed between said solid core and said drug-containing solid;
wherein
said first excipient is substantially mutually soluble with gastric fluid under physiological conditions and selected from the group consisting of hydroxypropyl methylcellulose, hydroxyethyl cellulose, polyvinyl alcohol, polyvinylpyrrolidone, sodium alginate, hydroxypropyl cellulose, hydroxyethyl cellulose, methyl cellulose, hydroxypropyl methyl ether cellulose, starch, polymethacrylates, poly(methacrylic acid, ethyl acrylate) 1:1, butylmethacrylat-(2-dimethylaminoethyl)methacrylat-methylmathacrylat-copolymer, polyethylene oxide, xanthan gum, or vinylpyrrolidone-vinyl acetate copolymer;
the solubility of gastric fluid in said second excipient is no greater than 700 mg/ml, and said second excipient is selected from the group consisting of methacrylic acid-ethyl acrylate copolymer, methacrylic acid-methyl methacrylate copolymer, ethyl acrylate-methylmethacrylate copolymer, hydroxypropyl methylcellulose acetate succinate, polyvinyl acetate, polymers including methacrylic acid, polymers including ethyl acrylate, polymers including methyl methacrylate, polymers including methacrylate, Poly[Ethyl acrylate, methyl methacrylate, trimethylammonioethyl methacrylate chloride], and ethylcellulose;
said third excipient comprises a solubility in gastric fluid greater than 1 mg/ml under physiological conditions and is selected from the group consisting of hydroxypropyl methylcellulose, hydroxyethyl cellulose, polyvinyl alcohol, polyvinylpyrrolidone, sodium alginate, hydroxypropyl cellulose, hydroxyethyl cellulose, methyl cellulose, hydroxypropyl methyl ether cellulose, starch, polymethacrylates, poly(methacrylic acid, ethyl acrylate) 1:1, butylmethacrylat-(2-dimethylaminoethyl)methacrylat-methylmathacrylat-copolymer, polyethylene oxide, or vinylpyrrolidone-vinyl acetate copolymer; and
upon immersing said pharmaceutical solid dosage form to gastric fluid under physiological conditions, the substantially encapsulated solid core expands with fluid absorption and said drug-containing solid releases said active ingredient over time.
